(19) United States
(12) Patent Application Publication
(10) Pub. No.: US 2011/0105959 A1
(43) Pub. Date: May 5, 2011

(54) Novel Methods for Bone Treatment by Modulating an Arachidonic Acid Metabolic or Signaling Pathway

(75) Inventor: James Patrick O'Connor, Newark, NJ (US)

(73) Assignee: ACCELALOX, INC., Menlo Park, CA (US)

(21) Appl. No.: 12/860,818

(22) Filed: Aug. 20, 2010

Related U.S. Application Data
(63) Continuation of application No. PCT/US2009/034790, filed on Feb. 20, 2009.
(60) Provisional application No. 61/030,764, filed on Feb. 22, 2008.

(51) Int. Cl. A61N 7/00; A61K 38/20; A61K 38/19; A61K 38/18; A61K 38/22; A61K 38/29; A61K 31/40; A61K 31/197; A61K 31/454; A61K 31/41; A61K 31/353; A61P 19/08; A61P 19/10

(52) U.S. Cl. 601/2; 424/85.2; 424/85.1; 514/8.9; 514/9.1; 514/9.6; 514/11.8; 514/8.8; 514/8.2; 514/423; 514/563; 514/321; 514/567; 514/381; 514/456; 514/428

(57) ABSTRACT

Methods for promoting osteogenesis to accelerate or enhance bone fracture healing, treat bone defects, and enhance bone formation are disclosed. The methods rely on in vivo or ex vivo modulation of an arachidonic acid metabolic or signaling pathway in general, and, in particular, utilize 5-lipoxygenase inhibitors, leukotriene A4 hydrolase inhibitors, and/or leukotriene B4 receptor antagonists. These molecules can be delivered alone or in combination with one or more agents that inhibit bone resorption, regulate calcium resorption from bone, enhance bone accumulation, enhance bone formation, induce bone formation, impair growth of microorganisms, reduce inflammation, and/or reduce pain.

### CROSS REFERENCE TO RELATED APPLICATIONS

This application is a continuation of International Application No. PCT/US2009/034790, filed Feb. 20, 2009 which claims the benefit of U.S. Provisional Application No. 61/030,764, filed Feb. 22, 2008. Each of these applications is incorporated by reference herein.

### FIELD OF INVENTION

The invention relates generally to accelerating or enhancing bone formation or fracture healing by modulating an arachidonic acid metabolic or signaling pathway, in particular by using inhibitors of 5-lipoxygenase activity, inhibitors of leukotriene A₄ hydrolase activity, and modifiers of leukotriene B₄ receptor activity.

### BACKGROUND OF THE INVENTION

Bone fractures are a common traumatic injury. Approximately 8-10 million bone fractures are reported annually in the United States with more than 1 million of these requiring hospitalization. The estimated annual cost of treating these fractures exceeds 20 billion dollars. While this is already significant, these numbers are expected to increase due to the aging of the general population. Further, among military personnel, bone fractures are common training injuries. Bone fractures, typically located in the arms and legs, are also common battle wounds. Aside from traumatic injury, bone fractures also can be caused by disease. Osteoporosis is caused by a reduction in bone mineral density in mature bone and results in fractures after minimal trauma. Osteoporosis is widespread and has a tremendous economic impact. The most common osteoporotic fractures occur in the vertebrae, distal radius and hip. An estimated one-third of the female population over age 65 will have vertebral fractures, caused in part by osteoporosis. Moreover, hip fractures are likely to occur in about one in every three woman and one in every six men by extreme old age.

Fracture healing is a complex tissue regeneration process that involves cell migration, proliferation, apoptosis, and differentiation in response to growth factors, cytokines, other signaling molecules, and the mechanical environment. The temporal order and magnitude of each cellular process must be controlled for optimal regeneration. The normal events of fracture healing are described below as occurring in 4 phases. In the initial phase, hematoma formation and localized tissue hypoxia are the initial cellular and molecular events of fracture healing. The second phase, called the early stage, is characterized by inflammation followed by rapid accumulation of cells at the fracture site. The presence of macrophages and neutrophils at the fracture site during inflammation precedes the rapid migration and proliferation of mesenchymal cells at the fracture site. In the third, regenerative phase, endochondral ossification creates the new bone which bridges the fracture. At this point, the fracture callus has a well-defined morphology. Intramembraneous ossification creates buttresses of periosteal bone at the callus periphery. Mesenchymal cells within the callus begin to differentiate into chondrocytes at the interface of the periosteal bone buttress. Each new chondrocyte develops as would be expected with matrix deposition followed by matrix calcification to produce calcified cartilage and then apoptosis. Channels are formed into the calcified cartilage starting at the periosteal bone buttresses. Osteoblasts migrate or differentiate on the surface of the calcified cartilage within these channels and begin depositing new bone. As chondrocyte differentiation proceeds from the periphery to the center of the callus (fracture site), channel formation, osteoblast differentiation, and new bone formation follows until the soft callus has been replaced with woven (immature) bone. Angiogenesis during the regenerative phase is essential. The immature woven bone created during the regenerative phase is mechanically unsuited for normal weight-bearing. To compensate for the decreased mechanical properties of the woven bone, the fracture callus has a significantly larger diameter which provides for greater structural mechanical properties. In the final, remodeling phase, fracture callus diameter diminishes until the bone obtains its normal dimensions while maintaining the bone's overall mechanical properties by enhancing material mechanical properties. This is accomplished by replacing the mechanically poor, woven bone with mechanically strong, lamellar (mature) bone. In successive rounds, osteoclasts resorb the woven bone and osteoblasts replace it with lamellar bone. Molecular mechanisms governing osteoclast formation and function occur through the RANKL-RANK pathway and this pathway is activated during fracture healing.

Fractures are generally treated conservatively by closed reduction of the fracture and immobilization (casting) of the affected bone. In such cases, the bone heals through the endochondral ossification pathway described above. Adequate nutrition, including vitamin C, vitamin D, and calcium, aids in healing. There has been no major advancement in the treatment of bone fractures since the mid 20^{th} century when open reduction and internal fixation of fractures became commonplace. The promise of growth factor treatments to enhance fracture healing has not been realized yet.

Unfortunately, many fractures require surgical intervention to increase healing success and reduce the likelihood of complication. There is only one approved pharmacological enhancement for bone healing and that is treatment with recombinant bone morphogenetic protein, either rhBMP-2 (Infuse) or rhBMP-7 (OP-1). Use of these growth factors requires surgery and due to expense and unknown potential side effects caused by the use of supraphysiological levels of growth factors, rhBMPs are used as a last-resort to heal recalcitrant fractures.

Autologous platelet-rich plasma (PRP) therapy is sometimes used to enhance bone formation by increasing the availability of growth factors at the fracture site. The natural initial repair response to fracture includes the formation of a blood clot and degranulation of platelets, which releases growth factors and cytokines at the fracture site, promoting activation and proliferation of local progenitor cells and thus facilitating the formation of new bone tissue. The growth factors found in the environment of a blood clot include platelet-derived growth factor (PDGF), epidermal growth factor (EGF), fibroblast growth factor-2 (FGF-2), transforming growth factor beta (TGF-beta), and vascular endothelial growth factors (VEGF). PDGF, EGF and FGF-2 have been shown to stimulate proliferation of osteoblast progenitors; TGF-beta increases matrix synthesis; and VEGF and FGF-2 potentially enhance angiogenesis and revascularization [Rai et al. Combination of platelet-rich plasma with polycaprolactone-tricalcium phosphate scaffolds for segmental defect repair. Journal of Biomedical Materials Research 81A:888-899 (2007)]. Preclinical and clinical studies of PRP therapy for enhancing bone formation indicate that considerable variability exists in the effectiveness of PRP therapy. Simman et al. investigated the efficacy of PRP treatment to accelerate fracture healing of rat femurs and their results suggested that PRP accelerates bone fracture healing in that animal model [Simman et al. Role of Platelet-Rich Plasma in Acceleration of Bone Fracture Healing. Annals of Plastic Surgery 61(3):337-344 (2008)]; while Li et al. and Weiner et al. concluded that use of PRP therapy adversely affected lumbar spine fusion in pigs and humans, respectively [Li et al. Anterior Lumbar Interbody Fusion with Carbon Fiber Cage Loaded with Bioceramics and Platelet-Rich Plasma: An Experimental Study on Pigs. European Spine Journal 13:354-358 (2004) and Weiner et al. Efficacy of Autologous Growth Factors in Lumber Intertransverse Fusions. Spine 28:1968-1970 (2003)]. Thus, methods to enhance the osteogenic activity of platelet-rich plasma are desirable.

There is significant investigation of the potential for autologous and allogenic mesenchymal stem cells, typically derived from bone marrow aspirate, to enhance bone formation. With appropriate activation, mesenchymal stem cells differentiate into chondrocytes and osteoblasts, precursors of cartilage and bone, respectively. Increasing the number of mesenchymal stem cells at the fracture site by administration of mesenchymal stem cell-ladened bone marrow aspirate is believed to enhance the body's ability to form new bone and heal a bone fracture. Clinical use of bone marrow aspirate to deliver mesenchymal stem cells to a fracture site has shown variable results. Methods to promote activation of mesenchymal stem cells and/or bone marrow aspirate include use of rhBMP or a substance believed to provide additional BMP to the fracture site, such as demineralized bone preparations or gene therapy approaches to express BMP. To address the absolute number of mesenchymal stem cells in the bone marrow aspirate sample, methods have been developed to purify, concentrate, and/or expand the number of mesenchymal stem cells prior to administration to the patient in need of bone formation. Thus, additional methods to enhance the osteogenic activity of mesenchymal stem cells and/or bone marrow aspirate are desirable.

Typical care of bone fracture patients also involves the administration of antibiotics, a narcotic, an NSAID, a COX-2 inhibitor or other pain killers during the healing process.

NSAIDs inhibit cyclooxygenase, thereby inhibiting the conversion of arachidonic acid into prostaglandins (e.g., PGD2, PGE2, PGF2α, PGI2, and TXA2). Arachidonic acid is also a precursor for the leukotrienes (LTB4, LTC4, LTD4, LTE4), lipoxins (LXA4, LXB4), and 5-hydroxyeicosatetraenoic acid (5-HETE). The enzyme 5-lipoxygenase (5-LO) converts arachidonic acid to 5-hydroperoxyeicosatetraenoic acid (5-HpETE). This is the first step in the metabolic pathway which yields 5-HETE, the leukotrienes (LTs), and the lipoxins. Leukotrienes are also pro-inflammatory with the ability to attract neutrophils and cause capillary permeability. The arachidonic acid metabolic pathway is summarized in FIG. 1.

Lipoxygenases are nonheme iron-containing enzymes found in plants and animals that catalyze the oxygenation of certain polyunsaturated fatty acids, such as lipids and lipoproteins. Several lipoxygenase enzymes are known, each having a characteristic oxidation action. Mammalian lipoxygenases are named by the position in arachidonic acid that is oxygenated. For example, the enzyme 5-lipoxygenase converts arachidonic acid to 5-hydroperoxyeicosatetraenoic acid (5-HpETE), while the enzyme 12-lipoxygenas converts arachidonic acid to 12-HpETE. The activity of 5-lipoxygenase requires a co-factor commonly called FLAP (five lipoxygenase activating protein). Leukotriene synthesis is reduced by drugs that inhibit FLAP (MK866) or mice lacking FLAP.

5-Lipoxygenase converts arachidonic acid into active metabolites as outlined in FIG. 1B. Arachidonic acid is converted in leukotriene A₄ (LTA4) in two steps that are both catalyzed by 5-lipoxygenase (5-LO). 5-HpETE is an intermediary metabolite of LTA4 synthesis and 5-HpETE can be converted into 5-HETE. LTA4 is converted into leukotriene B₄ (LTB4) which is the primary active metabolite of 5-LO. This conversion is catalyzed by leukotriene A₄ hydrolase (LTA4 hydrolase or LTA4-H). LTB4 acts through cell signaling mechanisms to affect cell physiology. The signaling is achieved through LTB4 interaction with either or both the BLT1 (LTBR) and BLT2 (LTBR2) leukotriene B4 receptors (LTB4 receptor).

LTA4 also is converted into the cysteinyl leukotrienes (leukotriene C4 (LTC4), leukotriene D4 (LTD4), and leukotriene E4 (LTE4)) by its initial conversion into LTC4 by LTC4 synthase. Thus by inhibition of LTA4-H or antagonism of the LTB4 receptor, production of the cysteinyl leukotrienes is not impaired. Therefore, inhibition of LTA4-H or antagonism of the LTB4 receptor is unlike inhibition of 5-LO or FLAP, which reduces synthesis of all leukotrienes (see FIG. 1B).

WO 95/30419 discloses that 5-LO inhibitors reduce osteoclast activity. The suppression of osteoclast activity inhibits bone resorption and reduces bone loss in human pathological conditions. Bone resorption is an integral part of fracture healing because it is necessary to remodel the newly formed woven bone into stronger, more mature lamellar bone. The inhibition of bone resorption would be expected to impair the later stages of normal fracture healing. Koivukangas et al., [Long-term administration of clodronate does not prevent fracture healing in rats. Clinical Orthopaedics and Related Research 408: 268-278 (2003)] and Peter et al. [Effect of alendronate on fracture healing and bone remodeling in dogs. Journal of Orthopaedic Research 14: 74-79 (1996)] disclose the effects of bisphosphonate therapy on fracture healing. Gerstenfeld et al. [Comparison of Effects of the bisphosphonate alendronate versus the RANKL inhibitor denosumab on murine fracture healing. Journal of Bone and Mineral Research 24: 196-208 (2009)] disclose the effects of bisphosphonate and an anti-RANKL monoclonal therapy (denosumab) on fracture healing. The data show that bisphosphonate therapy or anti-RANKL therapy which impair osteoclast activity and bone remodeling do not accelerate or inhibit the initial stages of fracture repair but do impair the later bone remodeling stage of healing. The bisphosphonate or denosumab effect on fracture healing reveals itself as persistence of a large fracture callus that contains mechanically immature, woven bone rather than mechanically mature, lamellar bone.

WO 03/066048 discloses that 12/15-lipoxygenase inhibitors can be used to prevent bone loss or increase bone mass. The publication describes data showing that bone mineral density is preserved in transgenic mice that over express IL-4 and that were treated with a 15-LO inhibitor. The publication does not disclose that 15-LO inhibitors can aid fracture healing or the treatment of non-unions. WO 03/066048 does not disclose methods for modulating an arachidonic acid metabolic or signaling pathway by inhibition of 5-LO, FLAP, LTA4-H, or the LTB4 receptor to accelerate and enhance bone formation.

Traianedes, K., et al., 5-Lipoxygenase metabolites inhibit bone formation in vitro. Endocrinology, 139: 3178-3184 (1998) discloses the effects of LTB4,5-HETE, and LTD4 (all products of 5-LO activity) on the differentiation of fetal rat calvaria (osteoblast) cells. The data show that 5-HETE and LTB4 reduce bone nodule formation and alkaline phosphatase activity in vitro but that LTD4 had no effect. The results from an in vitro organ culture model showed that LTB4 or 5-HETE treatment prevented a BMP2 induced increase in mouse calvaria thickness. The publication, however, does not disclose the use of any 5-LO, FLAP, LTA4-H, LTBR, or LTBR2 inhibitors, nor does it disclose that 5-LO, FLAP, LTA4-H, LTBR, and/or LTBR2 inhibition would lead to the same effect in cultured osteoblasts or in organ cultures. Similarly, Ren and Dziak, Effects of leukotrienes on osteoblast cell proliferation. Calcified Tissue International 49: 197-201 (1991) discloses that LTB4 treatment reduces proliferation of primary rat calvaria (osteoblast) cultures in vitro, but that LTB4 can promote proliferation of established osteoblast cell lines (Saos-2 and G292) in vitro at higher concentration (0.3-1 micromolar). Ren and Dziak also disclose that LTC4 had no effect on the proliferation of primary rat osteoblast cells or Saos-2 cells but did promote proliferation of G292 cells. Further, Ren and Dziak disclose that treatment of Saos-2 cells with a 5-LO inhibitor (AA-861) had no effect on Saos-2 cell proliferation. The publication indicates that 5-LO, FLAP, LTA4-H, LTBR, and/or LTBR2 inhibition should have no effect on osteogenesis.

Thus, it is readily apparent that compositions and methods for accelerating or enhancing bone formation or fracture healing would be highly desirable.

### SUMMARY

The present invention provides methods of promoting osteogenesis by administering a compound that reduces a 5-lipoxygenase activity to treat a bone fracture, a bone defect or a condition treated by inducing bone formation. In a related aspect, the compound is a 5-lipoxygenase activity-reducing compound disclosed herein.

In another aspect, the present invention provides methods of promoting osteogenesis by administering a compound that reduces a leukotriene A4 hydrolase activity to treat a bone fracture, a bone defect or a condition treated by inducing bone formation. In a related aspect, the compound is a leukotriene A4 hydrolase activity-reducing compound disclosed herein.

In yet another aspect, the present invention provides methods of promoting osteogenesis in a subject in need thereof by administering a compound that antagonizes a leukotriene B4 receptor activity, a 5-lipoxygenase activity, or a leukotriene A4 hydrolase activity, to treat a bone fracture, a bone defect or a condition treated by inducing bone formation. In a related aspect, the compound is selected from the group consisting of the leukotriene B4 receptor activity-reducing compounds, the 5-lipoxygenase activity-reducing compounds, and the leukotriene A4 hydrolase activity-reducing compounds described herein.

In various aspects, the bone fracture treated by the method is a non-osteoporotic fracture, an osteoporotic fracture, a fracture associated with a congenital disease, a fracture associated with an acquired disease, or an osteotomic fracture. In another aspect, the treated subject is receiving spinal fusion or joint arthrodesis treatment.

In another aspect of the invention, the methods can further comprise an additional active agent such as a modulator of the activity of a cyclooxygenase. In one aspect the activity of a cyclooxygenase-2 (COX-2) is increased (e.g., a compound selected from the group consisting of Prostaglandin E2, butaprost, sulprostone, CP-536, 745-01, CP-043,305-02, CP-044,519-02, CP432, ONO-4819, CP-533,536, prostaglandin F_{2α}, bimatoprost, cloprostenol, latanoprost, tafluprost, bone morphogenetic protein-2 (BMP2), platelet derived growth factor (PDGF), interleukin-1α, interleukin-1β, tumor necrosis factor-alpha (TNF-α), fibroblast growth factor (FGF), transforming growth factor-β (TGF-β), epidermal growth factor (EGF), parathyroid hormone (PTH), parathyroid hormone related peptide (PTHrP), teriparatide and derivatives, recombinant forms and mimetics of these compounds). In another aspect, the activity of cyclooxygenase-1 (COX-1) is reduced (e.g., a compound selected from the group consisting of SC-560, FR122047, Valeroyl salicylate, Aspirin, Dexketoprofene, Keterolac, Flurbiprofen, and Suprofen). In a related aspect, the subject is administered ultrasound therapy or exposed to a pulsed electromagnetic field in an amount sufficient to increase a COX-2 activity in said subject.

In another aspect, two or three or more compounds that reduce a leukotriene B4 activity, a 5-lipoxygenase, and/or a leukotriene A4 hydrolase are administered to promote osteogenesis in a subject in need of osteogenic treatment.

In another aspect, administration of an activity-reducing or antagonizing compound (e.g., a compound that reduces a 5-lipoxygenase activity, a compound that reduces a leukotriene A4 hydrolase activity, and/or a compound that antagonizes a leukotriene B4 receptor activity) is accomplished ex vivo by contacting a biological sample with the activity-reducing compound and administering the contacted sample to a subject. In a related aspect, the contacting of the biological sample by the activity-reducing compound occurs prior to the administration of the biological sample to the subject. In other aspects, the contacting of the biological sample with the activity-reducing or antagonizing compound occurs simultaneously with the administration of the biological sample to the subject, and/or after the biological sample has been positioned at the site targeted for treatment. In still another aspect, the biological sample can be contacted two or more times, e.g., at various times prior to, during, and/or after the administration of the biological sample to the subject being treated.

In one aspect, the biological sample is autologous to the subject. In another aspect, the biological sample is heterologous to the subject.

In one aspect, the biological sample comprises platelet rich plasma, bone marrow cells, or stem cells. In another related aspect, the stem cells are obtained from bone marrow, adipose tissue, skin tissue, placenta tissue, or umbilical cord blood tissue.

In certain aspects, the compound that reduces a leukotriene B4 activity is an inhibitor of a leukotriene A4 hydrolase activity. In certain aspects, the compound that reduces a leukotriene B4 activity is an antagonist of a leukotriene B4 receptor.

In one aspect, the compound that reduces a leukotriene B4 activity, a 5-lipoxygenase activity, and/or a leukotriene A4 hydrolase activity is a small molecule. In another aspect the compound is an antisense compound or an RNAi compound, e.g., one of the antisense or RNAi compounds described herein.

In yet another aspect, the invention provides a method wherein a subject is diagnosed with a bone fracture or bone defect in a subject or patient prior to the in vivo or ex vivo treatments described herein (e.g., the administration of an osteogenesis-promoting compound and/or the administration of a biological sample contacted with the compound). In a related aspect, a subject is diagnosed or determined to need enhanced or accelerated bone formation at a location in the subject's body, e.g., for cosmetic reasons, prior to the administration of the in vivo or ex vivo treatments described herein. In another aspect, bone repair or bone growth is measured or detected in said subject after administration of the osteogenic treatments described herein. In a related aspect, the status, rate, or extent of bone repair or bone growth achieved by the treatment is recorded or reported to technician, a physician treating the patient, and/or another party, e.g., the patient himself. In yet another aspect, additional treatment is provided to the subject or patient after the measurement, as necessary.

These and other aspects of the present invention will become evident upon reference to the following detailed description, the attached figures, and the claims. In addition, various references are set forth herein which describe in more detail certain procedures or compositions, and are therefore incorporated by reference in their entirety.

### BRIEF DESCRIPTION OF THE FIGURES

FIGS. 1A and 1B summarize exemplary arachidonic acid metabolic or signaling pathways.

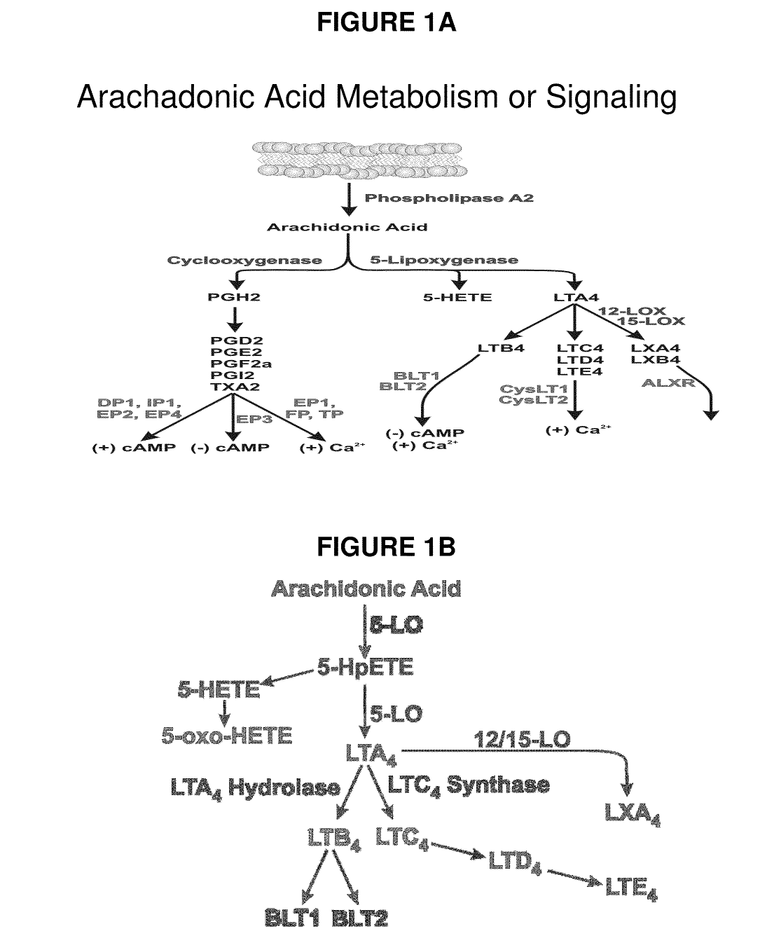

FIG. 2 illustrates the modulation of arachidonic acid metabolism by altering cyclooxygenase activity or lipoxygenase activity to accelerate or enhance bone formation. FIG. 2A represents the normal functioning of the pathway. FIG. 2B shows that the inhibition of COX-2 activity leads to excess leukotriene production which impairs bone formation in fracture healing or other osteogenic processes. FIG. 2C shows that the inhibition of lipoxygenase activity leads to excess prostaglandin production which accelerates or enhances bone formation in fracture repair or other osteogenic processes.

FIG. 3 shows serial x-rays of femur fractures made from a 5-LO−/− mouse and a normal mouse (C57BL/6). The x-rays show that osteogenesis, and therefore fracture healing, is accelerated in the 5-LO−/− mouse.

FIG. 4 illustrates mechanical testing data of fracture healing in wild-type (WT) and 5-LO knockout mice (5-LOKO or 5-LO−/−) 28 days and 84 days after the onset of the fracture. Peak torque (FIG. 4A), rigidity (FIG. 4B), maximum shear stress (FIG. 4C), and shear modulus (FIG. 4D) were calculated from callus dimensions and the torque to angular displacement curves.

FIG. 5 illustrates histomorphometric data of fracture healing from wild-type (WT, black bars) and 5-LO knockout mice (5-LOKO or 5-LO−/−, gray bars) at 7, 10, 14, and 21 days after fracture. The left panel shows the percent of fracture callus area that is newly formed bone (mineralized tissue) and the right panel shows the percent of fracture callus area that is cartilage.

FIG. 6 shows that fracture healing is dramatically impaired in COX-2 knock-out mice and that the defect in healing occurs because of lack of osteogenesis (new bone formation). FIG. 6A shows data from x-rays and FIGS. 6B and 6C show the histological samples of 14-day old femur fractures in mice lacking a functional COX-1 gene. FIG. 6D shows data from x-rays and FIGS. 6E and 6F show the histological samples of 14-day old femur fractures in mice lacking a functional COX-2 gene.

FIG. 7 illustrates that osteogenesis is accelerated in rats treated with 5-LO inhibitors, resulting in fractures healing faster than in untreated rats.

FIG. 8 illustrates that osteogenesis is accelerated in rats treated with two different 5-LO inhibitors, resulting in fractures healing faster than in untreated rats. FIGS. 8A, 8B, and 8C show data from x-rays for vehicle control (8A), 5-LO inhibitor NDGA (8B), and 5-LO inhibitor AA-861 (8C). FIG. 8D is a graph showing inhibition of 5-LO increases fracture callus peak torque.

FIG. 9 illustrates that ex vivo treatment of platelet-rich plasma with leukotriene pathway modifiers zileuton (a 5-LO inhibitor), AA-861 (a 5-LO inhibitor), MK-886 (a FLAP inhibitor) and SC-22716 (an LTA4 hydrolase inhibitor) prior to administration to patients in need of osteogenesis significantly reduces the activity of 5-lipoxygenase or LTA4 hydrolase, measured by the level of the LTB4 metabolite, in the platelet-rich plasma and, therefore, significantly reduces the extent to which administration of platelet-rich plasma supplies negative regulators of bone formation to patients in need of osteogenesis.

FIG. 10 shows more extensive bone remodeling at the fracture site of rats administered mesenchymal stem cell ladened bone marrow aspirate which, prior to administration, was treated with AA-861 (a 5-LO inhibitor), zileuton (a 5-LO inhibitor), SC-22716 (an LTA4 hydrolase inhibitor), LY-255283 (an LTB4 receptor antagonist) or the combination of AC-861, SC-22716 and LY-255283 compared to control rats, indicating that fracture healing progressed faster in the treated rats compared to the control rats.

FIG. 11 shows x-rays of femur fractures in an LTA4 hydrolase knockout (LTA4H-KO) mouse and a normal (wild type) mouse 2 weeks after fracture. The x-rays show radiographic bridging of the fracture with new bone in the LTA4H-KO mouse while the fracture site was not bridged at the same timepoint in the wild type mouse. This indicates that osteogenesis, and therefore fracture healing, is accelerated in the mouse lacking LTA4 hydrolase activity.

FIG. 12 shows the biomechanical properties of healing femurs from normal mice (wild type) and LTA4H-KO mice 4 weeks after fracture. Peak torque (FIG. 12A), maximum rigidity (FIG. 12B), maximum shear stress (FIG. 12C), and shear modulus (FIG. 12D) were calculated from callus dimensions and the torque to angular displacement curves. The data show enhanced fracture callus structural and material properties in LTA4H-KO mice compared to wild type. This demonstrates that osteogenesis is enhanced in the mice lacking LTA4 hydrolase activity.

FIG. 13 illustrates that osteogenesis is accelerated in rats treated with captopril, an LTA4 hydrolase inhibitor. FIG. 13A is a histological sample of a vehicle-treated rat callus at 4 weeks after fracture showing a normal callus that appears to be partially bridged with new bone on one side. FIG. 13B is a histological sample of a fracture callus from a captropril-treated rat after 4 weeks of healing showing a fully bridged callus that had already significantly remodeled based upon the smaller callus size and increased thickness of the callus peripheral bone. This demonstrates that pharmacological reduction of LTA4 hydrolase activity accelerates and enhances osteogenesis.

FIG. 14 shows histomorphometry analysis of the captropril-treated rats compared to the control rats. The rats treated with captopril (an LTA4 hydrolase inhibitor) show 66% less cartilage (P=0.040) and similar percentage of bone compared to the control rats. The near absence of cartilage in the treated rats indicates accelerated completion of the endochondral ossification phase of fracture healing and an overall acceleration in osteogenesis and fracture healing in the rats treated with the LTA4 hydrolase inhibitor.

FIG. 15 illustrates that osteogenesis is accelerated in rats treated with an LTA4 hydrolase inhibitor. FIG. 15A shows x-rays of femur fractures in rats 28-days post fracture. The rat treated with captopril, an LTA4 hydrolase inhibitor, shows complete bridging of the fracture and significant remodeling of the fracture callus, indicating nearly complete fracture healing; while the control rat shows a large fracture callus at 28-days post fracture. This shows that inhibition of LTA4 hydrolase accelerates and enhances osteogenesis. FIG. 15B shows the mean x-ray scores of the treated and control rats on a scale of 0 to 4 based upon apparent bone bridging across the callus at the left and right periphery (1 point each) and apparent bone bridging between the cortices of the femur on the left and right sides (1 point each) as described by Berkensotck et al (Bergenstock et al., A comparison between the effects of acetaminophen and celecoxib on bone fracture healing in rats. J. Orthop. Trauma. vol. 19, pages 717-723 (2005)). The captopril-treated rats had an average radiographic score of 3.6, higher than the average score of 3.0 for the control rats. These data indicate that inhibition of LTA4 hydrolase activity accelerates resolution of the fracture callus and thus accelerates osteogenesis and fracture healing.

FIG. 16 illustrates that healing of critical size segmental defects is enhanced and accelerated in rats treated with leukotriene pathway modifiers, including the 5-LO inhibitor AA-861, the FLAP inhibitor MK886, the LTA4 hydrolase inhibitor SC-22716 and the LTB4 receptor antagonist LY-255283. After 3 weeks of healing, x-rays of critical size segmental defects in the femurs of the rats treated with the leukotriene pathway modifiers show significantly more bone formation as evident by the x-ray dense material in the segmental defect region in all drug-treatment groups as compared to the scaffold-only group.

### DETAILED DESCRIPTION OF THE INVENTION

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3^{rd} Ed. (Plenum Press) Vols A and B (1992).

All publications, patents and patent applications cited herein, whether supra or infra, are hereby incorporated by reference in their entirety.

### I. Definitions

In describing the present invention, the following terms will be employed, and are intended to be defined as indicated below.

By “modulating an arachidonic acid metabolic or signaling pathway” is meant use of a drug or a compound which inhibits or promotes the activity or concentration of any enzyme or regulatory molecule involved in an arachidonic acid metabolism or signal pathway in a cell or animal. Preferably, a drug or a compound can be selected from a FLAP inhibitor such as BAYx 1005, MK-886, and MK-0591; a 5-Lipoxygenase inhibitor such as Zileuton, BAY-G576, RS-43,179, Wy-47,288, ABT-761, A-78773, A-79175, vitamin A, and BW A4C; a cysteinyl leukotriene receptor antagonist such as zafirlukast, montelukast, pranlukast, ICI 204,219, MK-571, MK-679, ONO—R^{S}-411, SK&F 104,353, and Wy-48,252; a leukotriene B4 receptor antagonists such as LY-255283, LY-223982, LY-293111, SB-201146, SB-225002, SC-41930, SC-53228, SC-50605, BIIL284, CP-105696, ONO-LB-457, and U-75302; leukotriene C4 synthase inhibitor; a Leukotriene A4 hydrolase inhibitor such as JNJ-26993135, SA-6541, SA-9499, SC-22716, SC-56938, SC-57461A, DG-051, 4-phenylchalcone oxide, captopril, bestatin, and kelatorphan; a non-steroidal anti-inflammatory drug (NSAID), a leukotriene receptor antagonists and leukotriene analogs, compounds modulating the formation and action of leukotrienes, compounds that affect cyclooxygenase activity, compounds that affect prostaglandin activity such as receptor agonists or antagonists, prostaglandin analogs, compounds that affect leukotriene activity such as receptor agonists or antagonists, and leukotriene analogs.

By “accelerated” is meant that osteogenesis occurs more rapidly and the time required for bone healing is reduced, or the bone heals more quickly in a treated subject as compared to an untreated subject or a control subject.

By “enhancing” is meant that the healed bone in the treated subject has improved characteristics compared to an untreated subject, or a control subject such as, for example, greater bone strength.

By “fracture healing” or “fracture repair” is meant that, in particular, promoting the healing of bone fractures and bone defects, and improving the mechanical stability of the healing fracture or site. Such bone fractures may be, for example, the common, traumatic (disabling and non-osteoporotic) fractures, the osteoporotic fractures due to osteoporosis or osteopenia of any etiology, fractures due to Paget's disease or fractures due to bone loss as a consequence of side effects of other drugs, e.g. in patients receiving high doses of corticosteroids, fractures arising from other congenital or acquired disease such as, e.g., osteogenesis imperfecta and breast cancer, surgical created fractures (osteotomies) used for example in bone lengthening and limb lengthening procedures, and treatment of bone fracture delayed unions or non-unions. The invention augments fracture healing following normal reduction and immobilization of the fracture using techniques common to one skilled in the art by accelerating and enhancing bone formation.

By “bone formation” is meant that the rate of bone formation in a subject treated according to the methods of the invention, such as, by receiving a 5-lipoxygenase inhibitor, a FLAP inhibitor, an LTA4 hydrolase inhibitor, an LTB4 receptor antagonist, and/or a biological sample that has been treated with a 5-LO inhibitor, a FLAP inhibitor, an LTA4 hydrolase inhibitor or an LTB4 receptor antagonist, is increased over the bone formation rate in a subject that is not given a 5-lipoxygenase inhibitor, a FLAP inhibitor, an LTA4 hydrolase inhibitor, an LTB4 receptor antagonist and/or a biological sample that has been treated with a 5-LO inhibitor, a FLAP inhibitor, an LTA4 hydrolase inhibitor or an LTB4 receptor antagonist. Such enhanced bone formation is determined herein using, e.g., quantitative digitized morphometry, as well as by other markers of bone formation, as described above. Bone formation is meant to include the osteogenic process used for spinal fusions and other joint or bone ankylosis application, bone formation into or around prosthetic devices, or bone formation to augment existing bones or replace missing bones or bone segments.

By “osteogenesis” is meant the production of bone that is associated with repair of a fractured bone, repair of a bone that has a defect caused by intentional or non-intentional damage, or induction of bone formation used to fuse more than one bone or bone segment together. “Osteogenesis” is not meant to include bone formation associated with normal bone growth in adolescents. “Osteogenesis” also is not meant to include bone formation associated with normal bone homeostasis, which is often referred to as bone remodeling, in which bone is normally turned-over by a process whereby osteoclasts resorb bone and osteoblasts make new bone to replace that which has been resorbed.

By “bone defect” is meant damage to a bone such that a portion of the bone is removed or is otherwise missing. Such bone defects would include anomalous holes, gaps or openings created in the bone for purposes of a diagnostic or therapeutic procedure, loss of bone segments from trauma or disease, puncture wounds to the bone, and the like.

The term “modulating” refers to the effect of a modulator on an arachidonic acid metabolic or signaling pathway. A modulator can be, e.g., a polypeptide, nucleic acid, macromolecule, complex molecule, small molecule, compound, or the like (naturally occurring or non-naturally occurring) that is capable of causing modulation. Modulators can be evaluated for potential activity as inhibitors or activators (directly or indirectly) of a functional property, biological activity or process, or a combination thereof (e.g., agonist, partial antagonist, partial agonist, inverse agonist, antagonist, and the like), by inclusion in assays that measure the activity of an enzyme in the pathway.

The terms “effective amount” or “pharmaceutically effective amount” refer to a sufficient amount of an agent to provide the desired biological result. That result can be reduction and/or alleviation of the signs, symptoms, or causes of a disease, or any other desired alteration of a biological system. For example, an “effective amount” for therapeutic uses is the amount of the composition comprising an active compound herein required to provide a clinically significant increase in osteogenesis and, thus, healing rates in fracture repair; stimulation and/or augmentation of bone formation in fracture non-unions, delayed unions and distraction osteogenesis; increase and/or acceleration of bone growth into prosthetic devices; enhanced or accelerated bone formation in joint ankylosis, bone ankylosis, or spinal fusions, bone formation to augment existing bone or replace missing bone or bone segments such as during incorporation of autograft, allograft, or synthetic bone material, and repair of dental defects.

As used herein, the terms “treat” or “treatment” are used interchangeably and are meant to indicate administering one or more compounds in accordance with the methods of the invention to promote osteogenesis to obtain a desired therapeutic objective. The terms further include ameliorating existing bone deficit symptoms, preventing additional symptoms, ameliorating or preventing the underlying metabolic causes of symptoms, and/or encouraging bone growth.

As used herein, “small molecule” is meant to indicate a chemical compound having a molecular weight of less than about 500 daltons. Small molecules do not include biologic polymers such as polypeptides and polynucleotides.

By “pharmaceutically acceptable” or “pharmacologically acceptable” is meant a material which is not biologically or otherwise undesirable, i.e., the material may be administered to an individual without causing any undesirable biological effects or interacting in a deleterious manner with any of the components of the composition in which it is contained.

By “physiological pH” or a “pH in the physiological range” is meant a pH in the range of approximately 7.2 to 8.0 inclusive, more typically in the range of approximately 7.2 to 7.6 inclusive.

As used herein, the term “subject” encompasses mammals. Examples of mammals include, but are not limited to, any member of the Mammalia class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. The term does not denote a particular age or gender.

The compounds of the present invention may be used to inhibit or reduce the activity of 5-lipoxygenase (5-LO), leukotriene A4 hydrolase (LTA4-H), leukotriene B4 receptors (BLT1 and/or BLT2), a combination of these activities, a combination of one or more of these activities and cyclooxygenase activity, and other enzymes and compounds in an arachadonic acid metabolic or signaling pathway. In this context, inhibition and reduction of the enzyme or receptor activity refers to a lower level of measured activity relative to a control experiment in which the enzyme, receptor, cell, or subject is not treated with the test compound. In particular embodiments, the inhibition or reduction in the measured activity is at least a 10% reduction or inhibition. One of skill in the art will appreciate that reduction or inhibition of the measured activity of at least 20%, 50%, 75%, 90% or 100% or any amount between 10% and 100%, may be preferred for particular applications. Inhibition of enzyme or receptor activity may be through any mechanism, including, by way of example, but not limitation, a reduction in the amount of enzyme present, a competitive or non-competitive inhibition of catalytic activity, an interference with an interaction between the enzyme and a co-factor or accessory protein, etc. In addition, the compounds of the present invention may be used to increase a cyclooxygenase activity. In particular embodiments, the increase of enzyme activity refers to a higher level of measured activity relative to a control experiment in which the enzyme, cell, or subject is not treated with the test compound. In particular embodiments, the increase in measured activity is at least a 10% increase. One of skill in the art will appreciate that an increase of the measured activity of at least 20%, 50%, 75%, 90% or 100% or any amount between 10% and 100% or beyond, may be preferred for particular applications. Increase of enzyme activity may be through any mechanism, including, by way of example but not limitation, an increase in the amount of enzyme present, or by increasing the enzyme's turnover rate, or altering its substrate binding properties.

References to the enzymes 5-lipoxygenase (5-LO), COX-1, COX-2, leukotriene A4 hydrolase (LTA4 hydrolase or LTA4-H), and to the leukotriene B4 receptor (BLT1 and/or BLT2) are intended to encompass the exemplary sequences referenced in Table 1, some of which are provided immediately following Table 1, as well as sequences at least 85% identical, 90% identical, or at least 95%, or at least 96%, or at least 97%, or at least 98%, or at least 99% identical to the exemplary sequences as can be ascertained by one of ordinary skill using routine alignment algorithms such as e.g., BLAST. In addition, other mammalian homologues are encompassed. Such homologues are identified as such on the basis of, e.g., sequence similarity, functional similarity, and by chromosome location. In addition to protein sequence, exemplary nucleic acid sequences are provided from which one of ordinary skill can readily obtain sequences of anti-sense and RNAi compounds useful for inhibiting the activity of the enzyme in accordance with the methods of the invention. Anti-sense compounds useful for practice of the invention are known in the art and can be obtained through commercial sources, as described in, e.g., Ding et al., (1999) BBRC vol. 261:1, pp. 218-223 (incorporated by reference).

TABLE 1
Exemplary Sequences

| | | | | | | |
|---|---|---|---|---|---|---|
| | | Entrez | GeneBank | | Protein | Similarity |
| | | Gene | Accession | mRNA | (Swiss- | to human |
| Symbol | OMIM ID | ID | Number | (GenBank) | Prot) | sequence |

Name: arachidonate 5-lipoxygenase; aka: 5-LO, 5-lipoxygenase

| | | | | | | | |
|---|---|---|---|---|---|---|---|
| Human | ALOX5 | 152390 | 240 | NC_000010 | NM_000698 | P09917 | NA |
| Rat | Alox5 | NA | 25290 | NC_005103 | NM_012822 | P12527 | 86.29% |
| | | | | | | | (n¹) |
| | | | | | | | 92.94% |
| | | | | | | | (p²) |
| Mouse | Alox5 | NA | 11689 | NC_000072 | NM_009662 | P48999 | 87.88% |
| | | | | | | | (n) |
| | | | | | | | 93.47% |
| | | | | | | | (p) |

Name: arachidonate 5-lipoxygenase-activating protein; aka: FLAP

| | | | | | | | |
|---|---|---|---|---|---|---|---|
| Human | ALOX5AP | 603700 | 241 | NC_000013 | NM_001629 | P20292 | NA |
| Rat | Alox5ap | NA | 29624 | NC_005111 | NM_017260 | P20291 | 85.09% |
| | | | | | | | (n) |
| | | | | | | | 91.93% |
| | | | | | | | (p) |
| Mouse | Alox5ap | NA | 11690 | NC_000071 | NM_009663 | P30355 | 85.71% |
| | | | | | | | (n) |
| | | | | | | | 91.93% |
| | | | | | | | (p) |

Name: prostaglandin-endoperoxide synthase 2; aka cyclooxygenase-2, COX-2,
PGHS-2

| | | | | | | | |
|---|---|---|---|---|---|---|---|
| Human | PTGS2 | 600262 | 5743 | NC_000001 | NM_000963 | P35354 | NA |
| Rat | Ptgs2 | NA | 29527 | NC_005112 | NM_017232 | P35355 | 83.17% |
| | | | | | | | (n) |
| | | | | | | | 84.91% |
| | | | | | | | (p) |
| Mouse | Ptgs2 | NA | 19225 | NC_000067 | NM_011198 | Q05769 | 84.71% |
| | | | | | | | (n) |
| | | | | | | | 86.75% |
| | | | | | | | (p) |

Name: prostaglandin-endoperoxide synthase 1; aka cyclooxygenase-1, COX-1,
PGHS-1

| | | | | | | | |
|---|---|---|---|---|---|---|---|
| Human | PTGS1 | 176805 | 5742 | NC_000009 | NM_000962 | P23219 | NA |
| Rat | Ptgs1 | NA | 24693 | NC_005102 | NM_017043 | Q63921 | 84.87% |
| | | | | | | | (n) |
| | | | | | | | 88.11% |
| | | | | | | | (p) |
| Mouse | Ptgs1 | NA | 19224 | NC_000068 | NM_008969 | P22437 | 85.59% |
| | | | | | | | (n) |
| | | | | | | | 89.78% |
| | | | | | | | (p) |

Name: Leukotriene A4 hydrolase; aka: LTA4 hydrolase, LTA4-H, LTA4H

| | | | | | | | |
|---|---|---|---|---|---|---|---|
| Human | LTA4H | 151570 | 4048 | BC032528 | NM_000895 | P09960 | NA |
| Rat | Lta4h | NA | 299732 | BC099819 | NM_00103003 | P001025202 | 81.42% |
| | | | | | | | (n) |
| | | | | | | | 85.01% |
| | | | | | | | (p) |
| Mouse | Lta4h | NA | 16993 | AK032828 | NM_008517 | P032543 | 82.77% |
| | | | | | | | (n) |
| | | | | | | | 83.11% |
| | | | | | | | (p) |

Name: Leukotriene B4 Receptor; aka: LTB4R, BLT1

| | | | | | | | |
|---|---|---|---|---|---|---|---|
| Human | LTB4R | 601531 | 1241 | NC_000014 | NM_181657 | Q15722 | NA |
| Rat | Ltb4r | NA | 59264 | AB025230 | NM_021656 | P067688 | 80.10% |
| | | | | | | | (n) |
| | | | | | | | 82.99% |
| | | | | | | | (p) |
| Mouse | Ltb4r1 | NA | 16995 | AF044030 | NM_008519 | P032545 | 84.27% |
| | | | | | | | (n) |
| | | | | | | | 87.05% |
| | | | | | | | (p) |

Name: Leukotriene B4 Receptor 2; aka: LTB4R2, BLT2

| | | | | | | | |
|---|---|---|---|---|---|---|---|
| Human | LTB4R2 | 605773 | 56413 | AB008193 | NM_019839 | Q9NPC1 | NA |
| Rat | Ltb4r2 | NA | 114098 | AB052660 | NM_053640 | P446092.1 | 83.13% |
| | | | | | | | (n) |
| | | | | | | | 82.09% |
| | | | | | | | (p) |
| Mouse | Ltb4r2 | NA | 57260 | AB029893 | NM_020490 | P065236.1 | 86.11% |
| | | | | | | | (n) |
| | | | | | | | 83.55% |
| | | | | | | | (p) |

¹Similarity between mRNA sequences.
²Similarity between protein sequences.

Human 5-Lipoxygenase mRNA Sequence (GenBank RefSeq NM_{—}000698)

### (SEQ ID NO: 1)

| | |
|---|---|
| 1 | gccagggacc agtggtggga ggaggctgcg gcgctagatg cggacacctg gaccgccgcg |
| 61 | ccgaggctcc cggcgctcgc tgctcccgcg gcccgcgcca tgccctccta cacggtcacc |
| 121 | gtggccactg gcagccagtg gttcgccggc actgacgact acatctacct cagcctcgtg |
| 181 | ggctcggcgg gctgcagcga gaagcacctg ctggacaagc ccttctacaa cgacttcgag |
| 241 | cgtggcgcgg tggattcata cgacgtgact gtggacgagg aactgggcga gatccagctg |
| 301 | gtcagaatcg agaagcgcaa gtactggctg aatgacgact ggtacctgaa gtacatcacg |
| 361 | ctgaagacgc cccacgggga ctacatcgag ttcccctgct accgctggat caccggcgat |
| 421 | gtcgaggttg tcctgaggga tggacgcgca aagttggccc gagatgacca aattcacatt |
| 481 | ctcaagcaac accgacgtaa agaactggaa acacggcaaa aacaatatcg atggatggag |
| 541 | tggaaccctg gcttcccctt gagcatcgat gccaaatgcc acaaggattt accccgtgat |
| 601 | atccagtttg atagtgaaaa aggagtggac tttgttctga attactccaa agcgatggag |
| 661 | aacctgttca tcaaccgctt catgcacatg ttccagtctt cttggaatga cttcgccgac |
| 721 | tttgagaaaa tctttgtcaa gatcagcaac actatttctg agcgggtcat gaatcactgg |
| 781 | caggaagacc tgatgtttgg ctaccagttc ctgaatggct gcaaccctgt gttgatccgg |
| 841 | cgctgcacag agctgcccga gaagctcccg gtgaccacgg agatggtaga gtgcagcctg |
| 901 | gagcggcagc tcagcttgga gcaggaggtc cagcaaggga acattttcat cgtggacttt |
| 961 | gagctgctgg atggcatcga tgccaacaaa acagacccct gcacactcca gttcctggcc |
| 1021 | gctcccatct gcttgctgta taagaacctg gccaacaaga ttgtccccat tgccatccag |
| 1081 | ctcaaccaaa tcccgggaga tgagaaccct attttcctcc cttcggatgc aaaatacgac |
| 1141 | tggcttttgg ccaaaatctg ggtgcgttcc agtgacttcc acgtccacca gaccatcacc |
| 1201 | caccttctgc gaacacatct ggtgtctgag gtttttggca ttgcaatgta ccgccagctg |
| 1261 | cctgctgtgc accccatttt caagctgctg gtggcacacg tgagattcac cattgcaatc |
| 1321 | aacaccaagg cccgtgagca gctcatctgc gagtgtggcc tctttgacaa ggccaacgcc |
| 1381 | acagggggcg gtgggcacgt gcagatggtg cagagggcca tgaaggacct gacctatgcc |
| 1441 | tccctgtgct ttcccgaggc catcaaggcc cggggcatgg agagcaaaga agacatcccc |
| 1501 | tactacttct accgggacga cgggctcctg gtgtgggaag ccatcaggac gttcacggcc |
| 1561 | gaggtggtag acatctacta cgagggcgac caggtggtgg aggaggaccc ggagctgcag |
| 1621 | gacttcgtga acgatgtcta cgtgtacggc atgcggggcc gcaagtcctc aggcttcccc |
| 1681 | aagtcggtca agagccggga gcagctgtcg gagtacctga ccgtggtgat cttcaccgcc |
| 1741 | tccgcccagc acgccgcggt caacttcggc cagtacgact ggtgctcctg gatccccaat |
| 1801 | gcgcccccaa ccatgcgagc cccgccaccg actgccaagg gcgtggtgac cattgagcag |
| 1861 | atcgtggaca cgctgcccga ccgcggccgc tcctgctggc atctgggtgc agtgtgggcg |
| 1921 | ctgagccagt tccaggaaaa cgagctgttc ctgggcatgt acccagaaga gcattttatc |
| 1981 | gagaagcctg tgaaggaagc catggcccga ttccgcaaga acctcgaggc cattgtcagc |
| 2041 | gtgattgctg agcgcaacaa gaagaagcag ctgccatatt actacttgtc cccagaccgg |
| 2101 | attccgaaca gtgtggccat ctgagcacac tgccagtctc actgtgggaa ggccagctgc |
| 2161 | cccagccaga tggactccag cctgcctggc aggctgtctg gccaggcctc ttggcagtca |
| 2221 | catctcttcc tccgaggcca gtacctttcc atttattctt tgatcttcag ggaactgcat |
| 2281 | agattgatca aagtgtaaac accataggga cccattctac acagagcagg actgcacagc |
| 2341 | gtcctgtcca cacccagctc agcatttcca caccaagcag caacagcaaa tcacgaccac |
| 2401 | tgatagatgt ctattcttgt tggagacatg ggatgattat tttctgttct atttgtgctt |
| 2461 | agtccaattc cttgcacata gtaggtaccc aattcaatta ctattgaatg aattaagaat |
| 2521 | tggttgccat aaaaataaat cagttcattt aaaaaaaaaa aaaaaaaa |

### Human 5-Lipoxygenase Protein Sequence (GenBank RefSeq NM_{—}000698)

### (SEQ ID NO: 2)

MPSYTVTVATGSQWFAGTDDYIYLSLVGSAGCSEKHLLDKPFYN
DFERGAVDSYDVTVDEELGEIQLVRIEKRKYWLNDDWYLKYITL
KTPHGDYIEFPCYRWITGDVEVVLRDGRAKLARDDQIHILKQHR
RKELETRQKQYRWMEWNPGFPLSIDAKCHKDLPRDIQFDSEKGV
DFVLNYSKAMENLFINRFMHMFQSSWNDFADFEKIFVKISNTIS
ERVMNHWQEDLMFGYQFLNGCNPVLIRRCTELPEKLPVTTEMVE
CSLERQLSLEQEVQQGNIFIVDFELLDGIDANKTDPCTLQFLAA
PICLLYKNLANKIVPIAIQLNQIPGDENPIFLPSDAKYDWLLAK
IWVRSSDFHVHQTITHLLRTHLVSEVFGIAMYRQLPAVHPIFKL
LVAHVRFTIAINTKAREQLICECGLFDKANATGGGGHVQMVQRA
MKDLTYASLCFPEAIKARGMESKEDIPYYFYRDDGLLVWEAIRT
FTAEVVDIYYEGDQVVEEDPELQDFVNDVYVYGMRGRKSSGFPK
SVKSREQLSEYLTVVIFTASAQHAAVNFGQYDWCSWIPNAPPTM
RAPPPTAKGVVTIEQIVDTLPDRGRSCWHLGAVWALSQFQENEL
FLGMYPEEHFIEKPVKEAMARFRKNLEAIVSVIAERNKKKQLPY
YYLSPDRIPNSVAI

Human FLAP mRNA Sequence (GenBank RefSeq NM_{—}001629)

### (SEQ ID NO: 3)

| | |
|---|---|
| 1 | acttcccctt cctgtacagg gcaggttgtg cagctggagg cagagcagtc ctctctgggg |
| 61 | agcctgaagc aaacatggat caagaaactg taggcaatgt tgtcctgttg gccatcgtca |
| 121 | ccctcatcag cgtggtccag aatggattct ttgcccataa agtggagcac gaaagcagga |
| 181 | cccagaatgg gaggagcttc cagaggaccg gaacacttgc ctttgagcgg gtctacactg |
| 241 | ccaaccagaa ctgtgtagat gcgtacccca ctttcctcgc tgtgctctgg tctgcggggc |
| 301 | tactttgcag ccaagttcct gctgcgtttg ctggactgat gtacttgttt gtgaggcaaa |
| 361 | agtactttgt cggttaccta ggagagagaa cgcagagcac ccctggctac atatttggga |
| 421 | aacgcatcat actcttcctg ttcctcatgt ccgttgctgg catattcaac tattacctca |
| 481 | tcttcttttt cggaagtgac tttgaaaact acataaagac gatctccacc accatctccc |
| 541 | ctctacttct cattccctaa ctctctgctg aatatggggt tggtgttctc atctaatcaa |
| 601 | tacctacaag tcatcataat tcagctcttg agagcattct gctcttcttt agatggctgt |
| 661 | aaatctattg gccatctggg cttcacagct tgagttaacc ttgcttttcc gggaacaaaa |
| 721 | tgatgtcatg tcagctccgc cccttgaaca tgaccgtggc cccaaatttg ctattcccat |
| 781 | gcattttgtt tgtttcttca cttatcctgt tctctgaaga tgttttgtga ccaggtttgt |
| 841 | gttttcttaa aataaaatgc agagacatgt ttt |

### Human FLAP Protein Sequence (GenBank RefSeq NM_{—}001629)

### (SEQ ID NO: 4)

MDQETVGNVVLLAIVTLISVVQNGFFAHKVEHESRTQNGRSFQRTG
TLAFERVYTANQNCVDAYPTFLAVLWSAGLLCSQVPAAFAGLMYLF
VRQKYFVGYLGERTQSTPGYIFGKRIILFLFLMSVAGIFNYYLIFF
FGSDFENYIKTISTTISPLLLIP

Human COX-2 mRNA Sequence (GenBank RefSeq NM_{—}00963)

### (SEQ ID NO: 5)

| | |
|---|---|
| 1 | caattgtcat acgacttgca gtgagcgtca ggagcacgtc caggaactcc tcagcagcgc |
| 61 | ctccttcagc tccacagcca gacgccctca gacagcaaag cctacccccg cgccgcgccc |
| 121 | tgcccgccgc tcggatgctc gcccgcgccc tgctgctgtg cgcggtcctg gcgctcagcc |
| 181 | atacagcaaa tccttgctgt tcccacccat gtcaaaaccg aggtgtatgt atgagtgtgg |
| 241 | gatttgacca gtataagtgc gattgtaccc ggacaggatt ctatggagaa aactgctcaa |
| 301 | caccggaatt tttgacaaga ataaaattat ttctgaaacc cactccaaac acagtgcact |
| 361 | acatacttac ccacttcaag ggattttgga acgttgtgaa taacattccc ttccttcgaa |
| 421 | atgcaattat gagttatgtc ttgacatcca gatcacattt gattgacagt ccaccaactt |
| 481 | acaatgctga ctatggctac aaaagctggg aagccttctc taacctctcc tattatacta |
| 541 | gagcccttcc tcctgtgcct gatgattgcc cgactccctt gggtgtcaaa ggtaaaaagc |
| 601 | agcttcctga ttcaaatgag attgtggaaa aattgcttct aagaagaaag ttcatccctg |
| 661 | atccccaggg ctcaaacatg atgtttgcat tctttgccca gcacttcacg catcagtttt |
| 721 | tcaagacaga tcataagcga gggccagctt tcaccaacgg gctgggccat ggggtggact |
| 781 | taaatcatat ttacggtgaa actctggcta gacagcgtaa actgcgcctt ttcaaggatg |
| 841 | gaaaaatgaa atatcagata attgatggag agatgtatcc tcccacagtc aaagatactc |
| 901 | aggcagagat gatctaccct cctcaagtcc ctgagcatct acggtttgct gtggggcagg |
| 961 | aggtctttgg tctggtgcct ggtctgatga tgtatgccac aatctggctg cgggaacaca |
| 1021 | acagagtatg cgatgtgctt aaacaggagc atcctgaatg gggtgatgag cagttgttcc |
| 1081 | agacaagcag gctaatactg ataggagaga ctattaagat tgtgattgaa gattatgtgc |
| 1141 | aacacttgag tggctatcac ttcaaactga aatttgaccc agaactactt ttcaacaaac |
| 1201 | aattccagta ccaaaatcgt attgctgctg aatttaacac cctctatcac tggcatcccc |
| 1261 | ttctgcctga cacctttcaa attcatgacc agaaatacaa ctatcaacag tttatctaca |
| 1321 | acaactctat attgctggaa catggaatta cccagtttgt tgaatcattc accaggcaaa |
| 1381 | ttgctggcag ggttgctggt ggtaggaatg ttccacccgc agtacagaaa gtatcacagg |
| 1441 | cttccattga ccagagcagg cagatgaaat accagtcttt taatgagtac cgcaaacgct |
| 1501 | ttatgctgaa gccctatgaa tcatttgaag aacttacagg agaaaaggaa atgtctgcag |
| 1561 | agttggaagc actctatggt gacatcgatg ctgtggagct gtatcctgcc cttctggtag |
| 1621 | aaaagcctcg gccagatgcc atctttggtg aaaccatggt agaagttgga gcaccattct |
| 1681 | ccttgaaagg acttatgggt aatgttatat gttctcctgc ctactggaag ccaagcactt |
| 1741 | ttggtggaga agtgggtttt caaatcatca acactgcctc aattcagtct ctcatctgca |
| 1801 | ataacgtgaa gggctgtccc tttacttcat tcagtgttcc agatccagag ctcattaaaa |
| 1861 | cagtcaccat caatgcaagt tcttcccgct ccggactaga tgatatcaat cccacagtac |
| 1921 | tactaaaaga acgttcgact gaactgtaga agtctaatga tcatatttat ttatttatat |
| 1981 | gaaccatgtc tattaattta attatttaat aatatttata ttaaactcct tatgttactt |
| 2041 | aacatcttct gtaacagaag tcagtactcc tgttgcggag aaaggagtca tacttgtgaa |
| 2101 | gacttttatg tcactactct aaagattttg ctgttgctgt taagtttgga aaacagtttt |
| 2161 | tattctgttt tataaaccag agagaaatga gttttgacgt ctttttactt gaatttcaac |
| 2221 | ttatattata agaacgaaag taaagatgtt tgaatactta aacactatca caagatggca |
| 2281 | aaatgctgaa agtttttaca ctgtcgatgt ttccaatgca tcttccatga tgcattagaa |
| 2341 | gtaactaatg tttgaaattt taaagtactt ttggttattt ttctgtcatc aaacaaaaac |
| 2401 | aggtatcagt gcattattaa atgaatattt aaattagaca ttaccagtaa tttcatgtct |
| 2461 | actttttaaa atcagcaatg aaacaataat ttgaaatttc taaattcata gggtagaatc |
| 2521 | acctgtaaaa gcttgtttga tttcttaaag ttattaaact tgtacatata ccaaaaagaa |
| 2581 | gctgtcttgg atttaaatct gtaaaatcag atgaaatttt actacaattg cttgttaaaa |
| 2641 | tattttataa gtgatgttcc tttttcacca agagtataaa cctttttagt gtgactgtta |
| 2701 | aaacttcctt ttaaatcaaa atgccaaatt tattaaggtg gtggagccac tgcagtgtta |
| 2761 | tctcaaaata agaatatttt gttgagatat tccagaattt gtttatatgg ctggtaacat |
| 2821 | gtaaaatcta tatcagcaaa agggtctacc tttaaaataa gcaataacaa agaagaaaac |
| 2881 | caaattattg ttcaaattta ggtttaaact tttgaagcaa actttttttt atccttgtgc |
| 2941 | actgcaggcc tggtactcag attttgctat gaggttaatg aagtaccaag ctgtgcttga |
| 3001 | ataacgatat gttttctcag attttctgtt gtacagttta atttagcagt ccatatcaca |
| 3061 | ttgcaaaagt agcaatgacc tcataaaata cctcttcaaa atgcttaaat tcatttcaca |
| 3121 | cattaatttt atctcagtct tgaagccaat tcagtaggtg cattggaatc aagcctggct |
| 3181 | acctgcatgc tgttcctttt cttttcttct tttagccatt ttgctaagag acacagtctt |
| 3241 | ctcatcactt cgtttctcct attttgtttt actagtttta agatcagagt tcactttctt |
| 3301 | tggactctgc ctatattttc ttacctgaac ttttgcaagt tttcaggtaa acctcagctc |
| 3361 | aggactgcta tttagctcct cttaagaaga ttaaaagaga aaaaaaaagg cccttttaaa |
| 3421 | aatagtatac acttatttta agtgaaaagc agagaatttt atttatagct aattttagct |
| 3481 | atctgtaacc aagatggatg caaagaggct agtgcctcag agagaactgt acggggtttg |
| 3541 | tgactggaaa aagttacgtt cccattctaa ttaatgccct ttcttattta aaaacaaaac |
| 3601 | caaatgatat ctaagtagtt ctcagcaata ataataatga cgataatact tcttttccac |
| 3661 | atctcattgt cactgacatt taatggtact gtatattact taatttattg aagattatta |
| 3721 | tttatgtctt attaggacac tatggttata aactgtgttt aagcctacaa tcattgattt |
| 3781 | ttttttgtta tgtcacaatc agtatatttt ctttggggtt acctctctga atattatgta |
| 3841 | aacaatccaa agaaatgatt gtattaagat ttgtgaataa atttttagaa atctgattgg |
| 3901 | catattgaga tatttaaggt tgaatgtttg tccttaggat aggcctatgt gctagcccac |
| 3961 | aaagaatatt gtctcattag cctgaatgtg ccataagact gaccttttaa aatgttttga |
| 4021 | gggatctgtg gatgcttcgt taatttgttc agccacaatt tattgagaaa atattctgtg |
| 4081 | tcaagcactg tgggttttaa tatttttaaa tcaaacgctg attacagata atagtattta |
| 4141 | tataaataat tgaaaaaaat tttcttttgg gaagagggag aaaatgaaat aaatatcatt |
| 4201 | aaagataact caggagaatc ttctttacaa ttttacgttt agaatgttta aggttaagaa |
| 4261 | agaaatagtc aatatgcttg tataaaacac tgttcactgt tttttttaaa aaaaaaactt |
| 4321 | gatttgttat taacattgat ctgctgacaa aacctgggaa tttgggttgt gtatgcgaat |
| 4381 | gtttcagtgc ctcagacaaa tgtgtattta acttatgtaa aagataagtc tggaaataaa |
| 4441 | tgtctgttta tttttgtact attta |

### Human COX-2 Protein Sequence (GenBank RefSeq NM_{—}000963)

### (SEQ ID NO: 6)

MLARALLLCAVLALSHTANPCCSHPCQNRGVCMSVGFDQYKCDCTR
TGFYGENCSTPEFLTRIKLFLKPTPNTVHYILTHFKGFWNVVNNIP
FLRNAIMSYVLTSRSHLIDSPPTYNADYGYKSWEAFSNLSYYTRAL
PPVPDDCPTPLGVKGKKQLPDSNEIVEKLLLRRKFIPDPQGSNMMF
AFFAQHFTHQFFKTDHKRGPAFTNGLGHGVDLNHIYGETLARQRKL
RLFKDGKMKYQIIDGEMYPPTVKDTQAEMIYPPQVPEHLRFAVGQE
VFGLVPGLMMYATIWLREHNRVCDVLKQEHPEWGDEQLFQTSRLIL
IGETIKIVIEDYVQHLSGYHFKLKFDPELLFNKQFQYQNRIAAEFN
TLYHWHPLLPDTFQIHDQKYNYQQFIYNNSILLEHGITQFVESFTR
QIAGRVAGGRNVPPAVQKVSQASIDQSRQMKYQSFNEYRKRFMLKP
YESFEELTGEKEMSAELEALYGDIDAVELYPALLVEKPRPDAIFGE
TMVEVGAPFSLKGLMGNVICSPAYWKPSTFGGEVGFQIINTASIQS
LICNNVKGCPFTSFSVPDPELIKTVTINASSSRSGLDDINPTVLLK
ERSTEL

Human COX-1 mRNA Sequence (GenBank RefSeq NM_{—}000962)

### (SEQ ID NO: 7)

| | |
|---|---|
| 1 | aggtgacagc tggagggagg agcgggggtg gagccggggg aagggtgggg aggggatggg |
| 61 | ctggagctcc gggcagtgtg cgaggcgcac gcacaggagc ctgcactctg cgtcccgcac |
| 121 | cccagcagcc gcgccatgag ccggagtctc ttgctctggt tcttgctgtt cctgctcctg |
| 181 | ctcccgccgc tccccgtcct gctcgcggac ccaggggcgc ccacgccagt gaatccctgt |
| 241 | tgttactatc catgccagca ccagggcatc tgtgtccgct tcggccttga ccgctaccag |
| 301 | tgtgactgca cccgcacggg ctattccggc cccaactgca ccatccctgg cctgtggacc |
| 361 | tggctccgga attcactgcg gcccagcccc tctttcaccc acttcctgct cactcacggg |
| 421 | cgctggttct gggagtttgt caatgccacc ttcatccgag agatgctcat gcgcctggta |
| 481 | ctcacagtgc gctccaacct tatccccagt ccccccacct acaactcagc acatgactac |
| 541 | atcagctggg agtctttctc caacgtgagc tattacactc gtattctgcc ctctgtgcct |
| 601 | aaagattgcc ccacacccat gggaaccaaa gggaagaagc agttgccaga tgcccagctc |
| 661 | ctggcccgcc gcttcctgct caggaggaag ttcatacctg acccccaagg caccaacctc |
| 721 | atgtttgcct tctttgcaca acacttcacc caccagttct tcaaaacttc tggcaagatg |
| 781 | ggtcctggct tcaccaaggc cttgggccat ggggtagacc tcggccacat ttatggagac |
| 841 | aatctggagc gtcagtatca actgcggctc tttaaggatg ggaaactcaa gtaccaggtg |
| 901 | ctggatggag aaatgtaccc gccctcggta gaagaggcgc ctgtgttgat gcactacccc |
| 961 | cgaggcatcc cgccccagag ccagatggct gtgggccagg aggtgtttgg gctgcttcct |
| 1021 | gggctcatgc tgtatgccac gctctggcta cgtgagcaca accgtgtgtg tgacctgctg |
| 1081 | aaggctgagc accccacctg gggcgatgag cagcttttcc agacgacccg cctcatcctc |
| 1141 | ataggggaga ccatcaagat tgtcatcgag gagtacgtgc agcagctgag tggctatttc |
| 1201 | ctgcagctga aatttgaccc agagctgctg ttcggtgtcc agttccaata ccgcaaccgc |
| 1261 | attgccatgg agttcaacca tctctaccac tggcaccccc tcatgcctga ctccttcaag |
| 1321 | gtgggctccc aggagtacag ctacgagcag ttcttgttca acacctccat gttggtggac |
| 1381 | tatggggttg aggccctggt ggatgccttc tctcgccaga ttgctggccg gatcggtggg |
| 1441 | ggcaggaaca tggaccacca catcctgcat gtggctgtgg atgtcatcag ggagtctcgg |
| 1501 | gagatgcggc tgcagccctt caatgagtac cgcaagaggt ttggcatgaa accctacacc |
| 1561 | tccttccagg agctcgtagg agagaaggag atggcagcag agttggagga attgtatgga |
| 1621 | gacattgatg cgttggagtt ctaccctgga ctgcttcttg aaaagtgcca tccaaactct |
| 1681 | atctttgggg agagtatgat agagattggg gctccctttt ccctcaaggg tctcctaggg |
| 1741 | aatcccatct gttctccgga gtactggaag ccgagcacat ttggcggcga ggtgggcttt |
| 1801 | aacattgtca agacggccac actgaagaag ctggtctgcc tcaacaccaa gacctgtccc |
| 1861 | tacgtttcct tccgtgtgcc ggatgccagt caggatgatg ggcctgctgt ggagcgacca |
| 1921 | tccacagagc tctgaggggc aggaaagcag cattctggag gggagagctt tgtgcttgtc |
| 1981 | attccagagt gctgaggcca gggctgatgg tcttaaatgc tcattttctg gtttggcatg |
| 2041 | gtgagtgttg gggttgacat ttagaacttt aagtctcacc cattatctgg aatattgtga |
| 2101 | ttctgtttat tcttccagaa tgctgaactc cttgttagcc cttcagattg ttaggagtgg |
| 2161 | ttctcatttg gtctgccaga atactgggtt cttagttgac aacctagaat gtcagatttc |
| 2221 | tggttgattt gtaacacagt cattctagga tgtggagcta ctgatgaaat ctgctagaaa |
| 2281 | gttagggggt tcttattttg cattccagaa tcttgacttt ctgattggtg attcaaagtg |
| 2341 | ttgtgttcct ggctgatgat ccagaacagt ggctcgtatc ccaaatctgt cagcatctgg |
| 2401 | ctgtctagaa tgtggatttg attcattttc ctgttcagtg agatatcata gagacggaga |
| 2461 | tcctaaggtc caacaagaat gcattccctg aatctgtgcc tgcactgaga gggcaaggaa |
| 2521 | gtggggtgtt cttcttggga cccccactaa gaccctggtc tgaggatgta gagagaacag |
| 2581 | gtgggctgta ttcacgccat tggttggaag ctaccagagc tctatcccca tccaggtctt |
| 2641 | gactcatggc agctgtttct catgaagcta ataaaattcg ctttctaaag ttacctgtta |
| 2701 | tatatctctt ttggtcccat cctctaaagc agaggcaaca ctggaacatg gctagccttt |
| 2761 | cttgtagcca tggctgggcg tgctagaggt tgcagcatga gactttctgc tgggatcctt |
| 2821 | gggcccatca ctgtatagac atgctaccac tggtacttcc tttctccctg cgggccaggc |
| 2881 | actgcccttt tcaggaagct ctcttaaaat acccattgcc ccagacctgg aagatataac |
| 2941 | attcagttcc caccatctga ttaaaacaac ttcctccctt acagagcata caacagaggg |
| 3001 | ggcacccggg gaggagagca catactgtgt tccaatttca cgcttttaat tctcatttgt |
| 3061 | tctcacacca acagtgtgaa gtgcgtggta taatctccat ttcaaaacca aggaagcagc |
| 3121 | ctcagagtgg tcgagtgaca cacctcacgc aggctgagtc cagagcttgt gctcctcttg |
| 3181 | attcctggtt tgactcagtt ccaggcctga tcttgcctgt ctggctcagg gtcaaagaca |
| 3241 | gaatggtgga gtgtagcctc cacctgatat tcaggctact cattcagtcc caaatatgta |
| 3301 | ttttcctaag tgtttactat gtgccagttc ctgtaacagg tgtggggaca cagcagtgag |
| 3361 | taatcaatac agacaaggtt ctgcccttat ggagctcaca ctccagtggc agacaaacag |
| 3421 | accataaata aggaaacgat gaaataagat atatacaagg tgagtgtgac ttcccttcta |
| 3481 | accccctctg ctctgtcctc ccctattgcg ctctcaagac cagagaccca acagcagtga |
| 3541 | tctcagggca gacagccctc cactccagct ctgagaccct tttctcagga cctctgtagg |
| 3601 | cagcagagag agaggacaga ggggtaagat gaggggttga gggaaggttc ttcatgatcc |
| 3661 | acactttggg cttagtattt ctcaggaaga gctatggccc agaaacaaca ggggaaacta |
| 3721 | gagttcggtc tgacagtcct tggggttaag tctcctgtct tatggtccag aaactcctgt |
| 3781 | ttctccttag ttggctggaa actgctccca tcattccttc tggcctctgc tgaatgcagg |
| 3841 | gaatgcaatc cttccctgct cttgcagttg ctctgacgta gaaagatcct tcgggtgctg |
| 3901 | gaagtctcca tgaagagctt gtgtcctgtc ctttcttgca gattctattt cccctcttct |
| 3961 | gctaatacct cttactttgc ttgagaatcc tctcctttct tattaatttc agtcttggtg |
| 4021 | gttctatcag gggtgcattc tggccaaggg gtgggcctgt gaatcaatcc tgggcaatca |
| 4081 | gacaccctct ccttaaaaac tggcccgtgg agactgagat cactgactct gactcatccc |
| 4141 | cacagctggc tctgacaaga tggtccattt gttcctgctt ccgagatccc cagggcagcc |
| 4201 | tggatccctg cccttctcaa gactttagct tttccttcca tccggtggcc tattccagga |
| 4261 | attcctcttt tgcttaaatc agttggagtt tgtgtctgtt gcttgtaatc aagcctttat |
| 4321 | ggctgctggg ctgagtgaca caagcacttt aatggcctgg agggactttt aatcagtgaa |
| 4381 | gatgcaatca gacaagtgtt ttggaaagag caccctcgag aagggtggat gacagggcag |
| 4441 | agcaggaagg acaggaagct ggcagaacgg aggaggctgc agccgtggtc caaccaggag |
| 4501 | ctgatggcag ctggggctag gggaagggct ttgagggtgg aaggatggga tgggttccag |
| 4561 | aggtattcct ctcttaaatg caagtgccta gattaggtag actttgctta gtattgacaa |
| 4621 | ctgcacatga aagttttgca aagggaaaca ggctaaatgc accaagaaag cttcttcaga |
| 4681 | gtgaagaatc ttaatgcttg taatttaaac atttgttcct ggagttttga tttggtggat |
| 4741 | gtgatggttg gttttatttg tcagtttggt tgggctatag cacacagtta tttaatcaaa |
| 4801 | cagtaatcta ggtgtggctg tgaaggtatt ttgtagatgt gattaacatc tacaatcagt |
| 4861 | tgactttaag tgaaagagat tacttaaata atttgggtga gctgcacctg attagttgaa |
| 4921 | aggcctcaag aacaaacact gcagtttcct ggaaaagaag aaactttgcc tcaagactat |
| 4981 | agccatcgac tcctgcctga gtttccagcc tgctagtctg ccctatggat ttgaagtttg |
| 5041 | ccaaccccaa caattgtgtg aattaatttc taaaaataaa gctatataca gcc |

### Human COX-1 Protein Sequence (GenBank RefSeq NM_{—}000962)

### (SEQ ID NO: 8)

MSRSLLLWFLLFLLLLPPLPVLLADPGAPTPVNPCCYYPCQHQGI
CVRFGLDRYQCDCTRTGYSGPNCTIPGLWTWLRNSLRPSPSFTHF
LLTHGRWFWEFVNATFIREMLMRLVLTVRSNLIPSPPTYNSAHDY
ISWESFSNVSYYTRILPSVPKDCPTPMGTKGKKQLPDAQLLARRF
LLRRKFIPDPQGTNLMFAFFAQHFTHQFFKTSGKMGPGFTKALGH
GVDLGHIYGDNLERQYQLRLFKDGKLKYQVLDGEMYPPSVEEAPV
LMHYPRGIPPQSQMAVGQEVFGLLPGLMLYATLWLREHNRVCDLL
KAEHPTWGDEQLFQTTRLILIGETIKIVIEEYVQQLSGYFLQLKF
DPELLFGVQFQYRNRIAMEFNHLYHWHPLMPDSFKVGSQEYSYEQ
FLFNTSMLVDYGVEALVDAFSRQIAGRIGGGRNMDHHILHVAVDV
IRESREMRLQPFNEYRKRFGMKPYTSFQELVGEKEMAAELEELYG
DIDALEFYPGLLLEKCHPNSIFGESMIEIGAPFSLKGLLGNPICS
PEYWKPSTFGGEVGFNIVKTATLKKLVCLNTKTCPYVSFRVPDAS
QDDGPAVERPSTEL

Human Leukotriene A4 Hydrolase mRNA Sequence

### (GenBank RefSeq NM_{—}000895)

### (SEQ ID NO: 86)

| | |
|---|---|
| 1 | ctctatcgac gagtctggta gctgagcgtt gggctgtagg tcgctgtgct gtgtgatccc |
| 61 | ccagagccat gcccgagata gtggatacct gttcgttggc ctctccggct tccgtctgcc |
| 121 | ggaccaagca cctgcacctg cgctgcagcg tcgactttac tcgccggacg ctgaccggga |
| 181 | ctgctgctct cacggtccag tctcaggagg acaatctgcg cagcctggtt ttggatacaa |
| 241 | aggaccttac aatagaaaaa gtagtgatca atggacaaga agtcaaatat gctcttggag |
| 301 | aaagacaaag ttacaaggga tcgccaatgg aaatctctct tcctatcgct ttgagcaaaa |
| 361 | atcaagaaat tgttatagaa atttcttttg agacctctcc aaaatcttct gctctccagt |
| 421 | ggctcactcc tgaacagact tctgggaagg aacacccata tctctttagt cagtgccagg |
| 481 | ccatccactg cagagcaatc cttccttgtc aggacactcc ttctgtgaaa ttaacctata |
| 541 | ctgcagaggt gtctgtccct aaagaactgg tggcacttat gagtgctatt cgtgatggag |
| 601 | aaacacctga cccagaagac ccaagcagga aaatatacaa attcatccaa aaagttccaa |
| 661 | taccctgcta cctgattgct ttagttgttg gagctttaga aagcaggcaa attggcccaa |
| 721 | gaactttggt gtggtctgag aaagagcagg tggaaaagtc tgcttatgag ttttctgaga |
| 781 | ctgaatctat gcttaaaata gcagaagatc tgggaggacc gtatgtatgg ggacagtatg |
| 841 | acctattggt cctgccacca tccttccctt atggtggcat ggagaatcct tgccttactt |
| 901 | ttgtaactcc tactctactg gcaggcgaca agtcactctc caatgtcatt gcacatgaaa |
| 961 | tatctcatag ctggacaggg aatctagtga ccaacaaaac ttgggatcac ttttggttaa |
| 1021 | atgagggaca tactgtgtac ttggaacgcc acatttgcgg acgattgttt ggtgaaaagt |
| 1081 | tcagacattt taatgctctg ggaggatggg gagaactaca gaattcggta aagacatttg |
| 1141 | gggagacaca tcctttcacc aaacttgtgg ttgatctgac agatatagac cctgatgtag |
| 1201 | cttattcttc agttccctat gagaagggct ttgctttact tttttacctt gaacaactgc |
| 1261 | ttggaggacc agagattttc ctaggattct taaaagctta tgttgagaag ttttcctata |
| 1321 | agagcataac tactgatgac tggaaggatt tcctgtattc ctattttaaa gataaggttg |
| 1381 | atgttctcaa tcaagttgat tggaatgcct ggctctactc tcctggactg cctcccataa |
| 1441 | agcccaatta tgatatgact ctgacaaatg cttgtattgc cttaagtcaa agatggatta |
| 1501 | ctgccaaaga agatgattta aattcattca atgccacaga cctgaaggat ctctcttctc |
| 1561 | atcaattgaa tgagttttta gcacagacgc tccagagggc acctcttcca ttggggcaca |
| 1621 | taaagcgaat gcaagaggtg tacaacttca atgccattaa caattctgaa atacgattca |
| 1681 | gatggctgcg gctctgcatt caatccaagt gggaggacgc aattcctttg gcgctaaaga |
| 1741 | tggcaactga acaaggaaga atgaagttta cccggccctt attcaaggat cttgctgcct |
| 1801 | ttgacaaatc ccatgatcaa gctgtccgaa cctaccaaga gcacaaagca agcatgcatc |
| 1861 | ccgtgactgc aatgctggtg gggaaagact taaaagtgga ttaaagacct gcgtattgat |
| 1921 | gattttagag atttctcttt tttaaatgga attcgtaaag aaatataaaa cttcagctca |
| 1981 | caattaaaac tgtcttttta gttttggctt tttattgttt tgttggtgat tttactgaaa |
| 2041 | taaagatgag ctacttcttc |

### Human Leukotriene A4 Hydrolase Protein Sequence (GenBank RefSeq NM_{—}000895) (SEQ ID NO: 87)

MPEIVDTCSLASPASVCRTKHLHLRCSVDFTRRTLTGTAALTVQSQED
NLRSLVLDTKDLTIEKVVINGQEVKYALGERQSYKGSPMEISLPIALS
KNQEIVIEISFETSPKSSALQWLTPEQTSGKEHPYLFSQCQAIHCRAI
LPCQDTPSVKLTYTAEVSVPKELVALMSAIRDGETPDPEDPSRKIYKF
IQKVPIPCYLIALVVGALESRQIGPRTLVWSEKEQVEKSAYEFSETES
MLKIAEDLGGPYVWGQYDLLVLPPSFPYGGMENPCLTFVTPTLLAGDK
SLSNVIAHEISHSWTGNLVTNKTWDHFWLNEGHTVYLERHICGRLFGE
KFRHFNALGGWGELQNSVKTFGETHPFTKLVVDLTDIDPDVAYSSVPY
EKGFALLFYLEQLLGGPEIFLGFLKAYVEKFSYKSITTDDWKDFLYSY
FKDKVDVLNQVDWNAWLYSPGLPPIKPNYDMTLTNACIALSQRWITAK
EDDLNSFNATDLKDLSSHQLNEFLAQTLQRAPLPLGHIKRMQEVYNFN
AINNSEIRFRWLRLCIQSKWEDAIPLALKMATEQGRMKFTRPLFKDLA
AFDKSHDQAVRTYQEHKASMHPVTAMLVGKDLKVD

Human Leukotriene B4 Receptor mRNA Sequence (GenBank RefSeq NM 181657) (SEQ ID NO: 88)

| | |
|---|---|
| 1 | ctggtcctgg gtgtggggaa gaaaggccat caaggtagat gcgggtgggg aacagcttga |
| 61 | gagaggaggc aaggacaacc cagtttctgt ctgaaggggc ctctggttga ccctggagtt |
| 121 | tctgtcccca aacacaggcc tcacgggatt ctttctgtcc tcatgcactg ggcagaggtt |
| 181 | ccttaacttc ctttgttgca cattgccatt ctctcacatc ccgtgcggtc aggaagccct |
| 241 | tcctgaactc tgacttcagt tcttgctgcg gtttctgccc atttttttca tatcctctga |
| 301 | cagctgcgag gtcatctctg ctctggcttt tctccaagca gaacaagtgg gggctctgga |
| 361 | aaggttaagg gacctcagtg gccaccatta tactttgcat ctttcctgag aagtgagagt |
| 421 | tgaaagggaa gcaggaaggc ccatggtcag attgaaggaa ggacttttta gtttcttttt |
| 481 | tttttttttt tttttttgag atggagtctc gctctgtcat tcaggctgga gtgcagtggt |
| 541 | gcgatctcag ctcactgcag cctccacttc ctgggttcac atgattctcc tgcctcagcc |
| 601 | tcccaagtag ctgagactac aggcacatgc cactacaccc agctatcttt tgtattttta |
| 661 | gtagagacgg ggtttcacca tgttggccag gctggtctca aactgctaac atcaagtgat |
| 721 | ctgctcccct cagcctccca aagtgctggg attaccggta tgaaccacca caacctgcca |
| 781 | ggaattttta gtttttagct tttgcaggag acttcaagga aaggagacat tcctctgtcc |
| 841 | aggaaacggg taaggggacc atttctgcat tgctggtttc ccctcttggc agggtgggca |
| 901 | tgaggcatca ctgttcctgc tccctcactc ctgctcctca tgctcagcct gccagctcgg |
| 961 | cctcaacttt gtgtgtctaa agtggaactg aatagtaggc tgtgagaaga taggaaagag |
| 1021 | gtagtgccaa tctccttgcc cagatcataa atccagactc agcagggtaa ccacatgggc |
| 1081 | aagcacaagg taggtgcttg gggaaagggg aagtaattgg cattctgtgt gataccaagg |
| 1141 | agaccatttg gattttggct tctaccaaag agaatggaga attggttgac ctaaatggaa |
| 1201 | ccagtccctt taagtaaggg gaggaaaggg ggtgctggaa gatggccctc ttcccaccac |
| 1261 | ctagatcata gcttgaactg aagccaagga cagagtgctg cccccttcgg catttactga |
| 1321 | tgtgccctct ttaaatcatg atgttatcta acccaaaccc agacccagga cctagtcaca |
| 1381 | gctccaacct acacttccta ttaatcttaa aacaaagcaa aacaaaacaa aaagatatca |
| 1441 | gcattgtagc ctccaatctg agcccatttc ccttctctgg ctaccatacc tccttctcct |
| 1501 | atatgatacc attcactact ttgttcaatt atccagtcta gacctgcatc ttgaggccac |
| 1561 | acccagcctt ctcactcccc acacccctct ttcctctctc actgctcctt cctggtctct |
| 1621 | tctcatctgg ccccacctct aaggagtcct cctgccttct gggttgccct ggaaaacaga |
| 1681 | ctatcccccc tcctagtgaa gggagtgggt aggggtttca gccccaccct caggaagatg |
| 1741 | cgtcttccct gtcctctgct ctgtggtact tcctctctgg ctgatttagc aaacagcacc |
| 1801 | tagacctggg gccaggcctt tggcagtggg acagatccag ggataggcta caccaccctg |
| 1861 | ccctgaccct gggattggca tcagcttcca accagttcct gccaaagctt gtaagtcctc |
| 1921 | ccgacggcca tgaacactac atcttctgca gcacccccct cactaggtgt agagttcatc |
| 1981 | tctctgctgg ctatcatcct gctgtcagtg gcgctggctg tggggcttcc cggcaacagc |
| 2041 | tttgtggtgt ggagtatcct gaaaaggatg cagaagcgct ctgtcactgc cctgatggtg |
| 2101 | ctgaacctgg ccctggccga cctggccgta ttgctcactg ctcccttttt ccttcacttc |
| 2161 | ctggcccaag gcacctggag ttttggactg gctggttgcc gcctgtgtca ctatgtctgc |
| 2221 | ggagtcagca tgtacgccag cgtcctgctt atcacggcca tgagtctaga ccgctcactg |
| 2281 | gcggtggccc gcccctttgt gtcccagaag ctacgcacca aggcgatggc ccggcgggtg |
| 2341 | ctggcaggca tctgggtgtt gtcctttctg ctggccacac ccgtcctcgc gtaccgcaca |
| 2401 | gtagtgccct ggaaaacgaa catgagcctg tgcttcccgc ggtaccccag cgaagggcac |
| 2461 | cgggccttcc atctaatctt cgaggctgtc acgggcttcc tgctgccctt cctggctgtg |
| 2521 | gtggccagct actcggacat agggcgtcgg ctacaggccc ggcgcttccg ccgcagccgc |
| 2581 | cgcaccggcc gcctggtggt gctcatcatc ctgaccttcg ccgccttctg gctgccctac |
| 2641 | cacgtggtga acctggctga ggcgggccgc gcgctggccg gccaggccgc cgggttaggg |
| 2701 | ctcgtgggga agcggctgag cctggcccgc aacgtgctca tcgcactcgc cttcctgagc |
| 2761 | agcagcgtga accccgtgct gtacgcgtgc gccggcggcg gcctgctgcg ctcggcgggc |
| 2821 | gtgggcttcg tcgccaagct gctggagggc acgggctccg aggcgtccag cacgcgccgc |
| 2881 | gggggcagcc tgggccagac cgctaggagc ggccccgccg ctctggagcc cggcccttcc |
| 2941 | gagagcctca ctgcctccag ccctctcaag ttaaacgaac tgaactaggc ctggtggaag |
| 3001 | gaggcgcact ttcctcctgg cagaatgcta gctctgagcc agttcagtac ctggaggagg |
| 3061 | agcaggggcg tggagggcgt ggagggcgtg ggagcgtggg aggcgggagt ggagtggaag |
| 3121 | aagagggaga ggtggagcaa agtgagggcc gagtgagagc gtgctccagc ctggctccca |
| 3181 | caggcagctt taaccattaa aactgaagtc tgaaatttgg tcaaccttgt gagtggggta |
| 3241 | catgtgctgt gggtatcggg gtgctcgtgg gcgccctggt ggggcccctc tcggtagttg |
| 3301 | agagtcacgt cctttagttc cccatgattt acaattttgg aagggacaca aagaaacata |
| 3361 | gacttccccc atcccagatg attccgagta catagtctgc agataatact tagcaaaacg |
| 3421 | cagtctacag actcctaaag cagcttgtct aggaagacca cccatgtggg cttatcactc |
| 3481 | caggttctgt gacccgggac cttctgagaa aacagcactg ctgtgaaata tcttccttga |
| 3541 | agcctgtgat aagtctcctt gttagaatga ctccaacttc ctgccaataa tctttgtcct |
| 3601 | ctccatagga gatgttctag gggatgcctt ccttccctcc atttcacaaa gaggccagac |
| 3661 | ttgaggacta agtcattaga tcttatccct tagaattttg catatcagca tctgctaacc |
| 3721 | tccacaacac accctggcac agggtggggc tgagggcccc aggaaacaaa gattcccaaa |
| 3781 | agtgagaggg atgagtcatt atttcctaga gatgactgtt gttttagaga accttggtcc |
| 3841 | aactctgttc tgacaaggtt ttaggaagat ggcaacaaca gtggcagcag tgtacttttt |
| 3901 | ggatctttct cataaaaaaa caaaagagca agtaagagag ggaaaccaaa tatccacatg |
| 3961 | caacatccgc aacaaatcta gtatgtcaag gtttgacata ctcccatgga ccccaaagta |
| 4021 | tgagccagtg agaatgagtc atcaatatct caagacccat ataccagcat ctgtgcagaa |
| 4081 | ggatgcagaa ggaagcaaag ggatgttgga tggacctaag aagaggagat ccc |

### Human Leukotriene B4 Receptor Protein Sequence (GenBank RefSeq NM_{—}181657)

### (SEQ ID NO: 89)

MNTTSSAAPPSLGVEFISLLAIILLSVALAVGLPGNSFVVWSILKRM
QKRSVTALMVLNLALADLAVLLTAPFFLHFLAQGTWSFGLAGCRLCH
YVCGVSMYASVLLITAMSLDRSLAVARPFVSQKLRTKAMARRVLAGI
WVLSFLLATPVLAYRTVVPWKTNMSLCFPRYPSEGHRAFHLIFEAVT
GFLLPFLAVVASYSDIGRRLQARRFRRSRRTGRLVVLIILTFAAFWL
PYHVVNLAEAGRALAGQAAGLGLVGKRLSLARNVLIALAFLSSSVNP
VLYACAGGGLLRSAGVGFVAKLLEGTGSEASSTRRGGSLGQTARSGP
AALEPGPSESLTASSPLKLNELN

Human Leukotriene B4 Receptor-2 mRNA Sequence

### (GenBank RefSeq NM_{—}019839)

### (SEQ ID NO: 90)

| | |
|---|---|
| 1 | aaactggccc tggccctgaa ccaaatacct tgaaccctcg taaactccat accctgaccc |
| 61 | ccttgttttg gatataccca ggtagaacaa ctctctctca ctgtctgttg tgaggatacg |
| 121 | ctgtagccca ctcattaagt acattctcct aataaatgct ttggactgat caccctgcca |
| 181 | gtcttttgtc ttgggcaatc tatacttttc tcagaggttc ccaaggccta ctgaagggac |
| 241 | ttaacatact cttaatggct ttcctctctc ttgttttacc ttatgccctc acttcctgag |
| 301 | ttaacctccc aaatacagga tcacctgtac ccaagccctt agctcaagaa tacaggatca |
| 361 | cctgtaccca agcccttagc tcaagctctg ctttggaaga acccaaacta agacagtgct |
| 421 | cctggtgccc tccccaagca acctcaagtt ctggctgtta cttgagcaga ggcctttctt |
| 481 | ttcccttccc ccagctctat ccatctgcca ggcccccctc aaatctcttc atttccaagt |
| 541 | tttgcttgac ttttccaaga ggagagggct gcttcttagt atgtccctac tcatcctttc |
| 601 | ctttcttgtc ttgtatcctg gtgcagcctg gtaatggggc ctcttcatgg ttgtgtgtca |
| 661 | tgactcccta accattatgc ctccatgcat cccctgttcc tcctggaacc tagcaccatg |
| 721 | ccttacatgg aaaagctgtc attgacagcc cggtgagagc cctgagggtg gagtgactgg |
| 781 | ggcagggcct gaggcaagag gtgggaggag gtaggaggcc aggggctcag ccggaccagg |
| 841 | agactggaaa caggcaagga taaggcaggt gggggactga gttgtttggg tcacctctgc |
| 901 | aggccagaga gaccaggcaa catacacact gcagaaggtg ggctgggagg attggggcca |
| 961 | gagctggggg agggatgaga acagaagcag gaccaggatt cagcagagtc ctcctatttc |
| 1021 | cttccaccac cagggaatct tactgcccca cttcagcttg tgctgtttcc tggcaaggca |
| 1081 | ggctctcaca tgcctggacg cctgggtgcg ttggtgatgg gaaggagcag ggtgagggag |
| 1141 | gggccccagg agaggcccag gatgagcctc atcttgtccc tccccattct tgtcttaccc |
| 1201 | tctgcaaatg tgataggcac aggacaggag taggcacctc gcctactgct gcttaacctt |
| 1261 | tcagcttctc caggccccca atcctgcttg ctcccagctt ggtaagtaga tctgtgcacg |
| 1321 | tccctttaca ccccaccatc cagttttgcc cagatgtgct agaatggggc tggacaaaga |
| 1381 | aggaggggcc agactagagg agtggtggta gagatagtga cagcctgggg tgaggacttt |
| 1441 | atgcctgttt accactgagc tctgggaagg aggccaggag tggggcaggt caactgactg |
| 1501 | ggagcagggg atctgggttc caagaaggag ttgtgtttga ggtggggtct gggtcctcgt |
| 1561 | ggaagtcagg actcccaggc agaaaagagg caggctgcag ggaagtaagg aggaggcatg |
| 1621 | gcaccttctc atcgggcatc acaggtgggg ttttgcccca cccctgaacg ccctctgtgg |
| 1681 | cgccttccac ccacctgtag gcccagaagg atgtcggtct gctaccgtcc cccagggaac |
| 1741 | gagacactgc tgagctggaa gacttcgcgg gccacaggca cagccttcct gctgctggcg |
| 1801 | gcgctgctgg ggctgcctgg caacggcttc gtggtgtgga gcttggcggg ctggcggcct |
| 1861 | gcacgggggc gaccgctggc ggccacgctt gtgctgcacc tggcgctggc cgacggcgcg |
| 1921 | gtgctgctgc tcacgccgct ctttgtggcc ttcctgaccc ggcaggcctg gccgctgggc |
| 1981 | caggcgggct gcaaggcggt gtactacgtg tgcgcgctca gcatgtacgc cagcgtgctg |
| 2041 | ctcaccggcc tgctcagcct gcagcgctgc ctcgcagtca cccgcccctt cctggcgcct |
| 2101 | cggctgcgca gcccggccct ggcccgccgc ctgctgctgg cggtctggct ggccgccctg |
| 2161 | ttgctcgccg tcccggccgc cgtctaccgc cacctgtgga gggaccgcgt atgccagctg |
| 2221 | tgccacccgt cgccggtcca cgccgccgcc cacctgagcc tggagactct gaccgctttc |
| 2281 | gtgcttcctt tcgggctgat gctcggctgc tacagcgtga cgctggcacg gctgcggggc |
| 2341 | gcccgctggg gctccgggcg gcacggggcg cgggtgggcc ggctggtgag cgccatcgtg |
| 2401 | cttgccttcg gcttgctctg ggccccctac cacgcagtca accttctgca ggcggtcgca |
| 2461 | gcgctggctc caccggaagg ggccttggcg aagctgggcg gagccggcca ggcggcgcga |
| 2521 | gcgggaacta cggccttggc cttcttcagt tctagcgtca acccggtgct ctacgtcttc |
| 2581 | accgctggag atctgctgcc ccgggcaggt ccccgtttcc tcacgcggct cttcgaaggc |
| 2641 | tctggggagg cccgaggggg cggccgctct agggaaggga ccatggagct ccgaactacc |
| 2701 | cctcagctga aagtggtggg gcagggccgc ggcaatggag acccgggggg tgggatggag |
| 2761 | aaggacggtc cggaatggga cctttgacag cagaccct |

### Human Leukotriene B4 Receptor-2 Protein Sequence (GenBank RefSeq NM_{—}019839) (SEQ ID NO: 91)

MAPSHRASQVGFCPTPERPLWRLPPTCRPRRMSVCYRPPGNETLLSWK
TSRATGTAFLLLAALLGLPGNGFVVWSLAGWRPARGRPLAATLVLHLA
LADGAVLLLTPLFVAFLTRQAWPLGQAGCKAVYYVCALSMYASVLLTG
LLSLQRCLAVTRPFLAPRLRSPALARRLLLAVWLAALLLAVPAAVYRH
LWRDRVCQLCHPSPVHAAAHLSLETLTAFVLPFGLMLGCYSVTLARLR
GARWGSGRHGARVGRLVSAIVLAFGLLWAPYHAVNLLQAVAALAPPEG
ALAKLGGAGQAARAGTTALAFFSSSVNPVLYVFTAGDLLPRAGPRFLT
RLFEGSGEARGGGRSREGTMELRTTPQLKVVGQGRGNGDPGGGMEKDG
PEWDL

### II. Arachidonic Acid Pathway Modulating Compounds

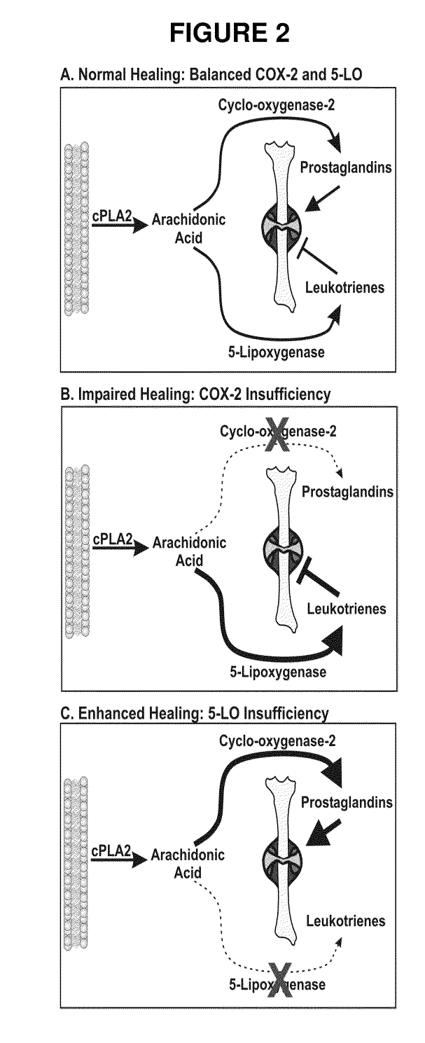

The applicant has discovered a method of inhibiting the activity of 5-lipoxygenase, LTA4 hydrolase and/or LTB4 receptors to promote osteogenesis, accelerates and/or enhances the healing of a bone fracture, accelerates and/or enhances the treatment of a bone defect, and accelerates and/or enhances bone formation. During a normal inflammation response, such as a fracture, the synthesis of prostaglandins and leukotrienes is balanced (FIG. 2A). Without being bound to a theory, inhibiting COX-2 function appears to shunt arachidonic acid into the lipoxygenase pathway to produce excess leukotrienes thereby impairing bone formation (FIG. 2B). Applicant has discovered that inhibiting 5-lipoxygenase activity shunts arachidonic acid into the cyclooxygenase pathway to produce excess prostaglandins that accelerate or enhance bone formation (FIG. 2C).

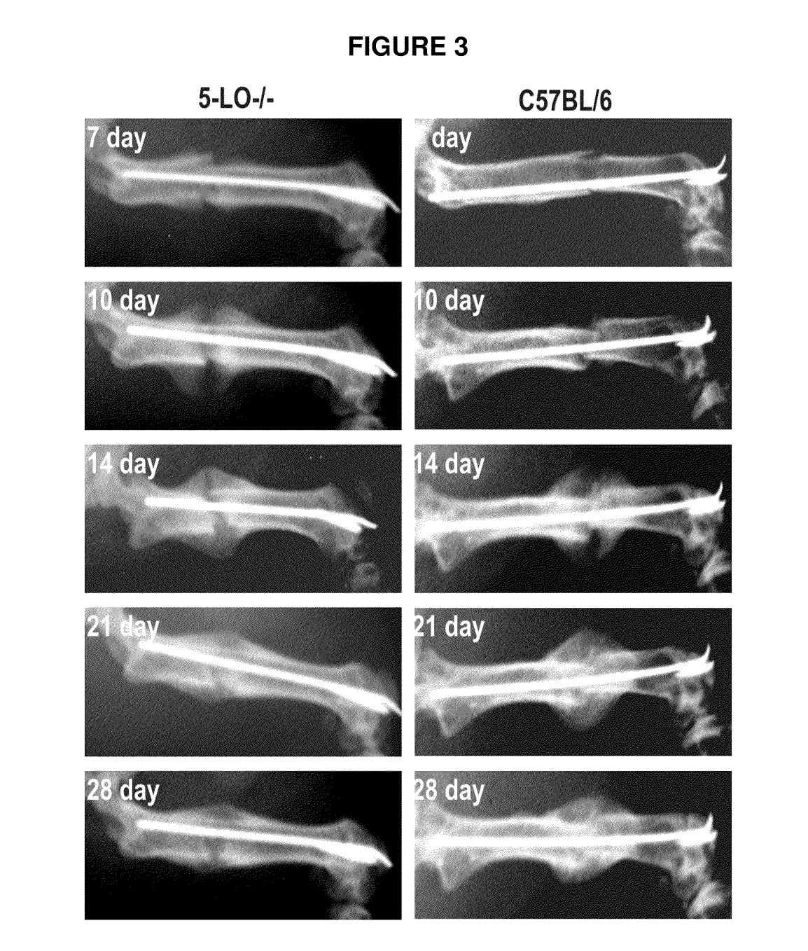

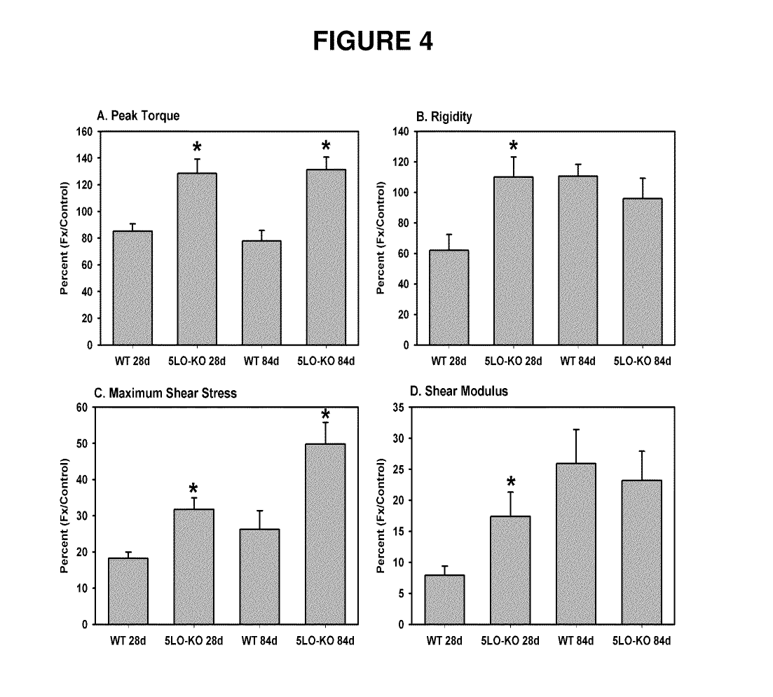

To test this potential mechanism, fracture healing was assessed in 5-LO−/− mice. Radiographic examination of fracture healing in age-matched mice in the C57BL/6 background showed that fracture bridging occurred by 2 weeks post-fracture in the 5-LO−/− mice as compared to 3 weeks post-fracture in the normal mice (FIG. 3). Further, callus remodeling was significantly accelerated, thus the 5-LO−/− callus regains its initial structural and material properties much faster than in normal mice based upon torsional mechanical testing (FIG. 4 and TABLE 2). Thus, the loss of 5-LO function accelerates and enhances fracture healing and bone formation.

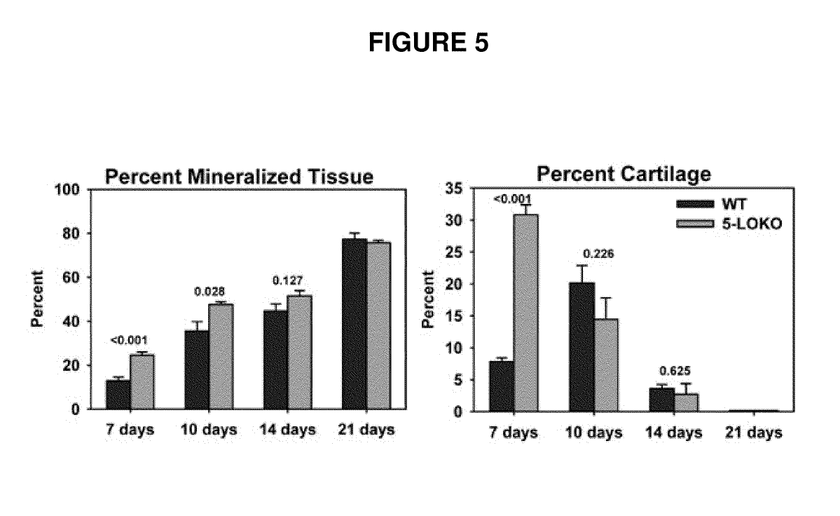

Histological examination of calcified samples supported the radiographic data. Plastic embedded, calcified sections of normal and 5-LO−/− mouse fractures stained with Stevenel's blue and van Gieson's picrofuchsin show that after just 2 weeks of healing the fracture was bridged with calcified tissue in the 5-LO−/− mice while the normal mouse (C57BL/6) still had a cartilaginous soft callus. Histomorphometric measurements of fracture callus cartilage area showed that cartilage area peaked by day 7 post-fracture in 5-LO−/− mice and by day 10 post-fracture in normal mice (FIG. 5 and TABLE 3). Measurement of new bone (calcified tissue) in the fracture callus showed that almost twice as much new bone in the 5-LO−/− after 7 days of healing and significantly more new bone at day 10 as well (FIG. 5 and TABLE 3). These data show that a normal, albeit significantly accelerated, endochondral ossification pathway is used to heal the fracture in the 5-LO−/− mice. Experiments using younger and older 5-LO−/− mice and in different genetic backgrounds gave identical results: loss of 5-LO function results in accelerated bone regeneration.

The data from these experiments show that a 10 day fracture callus in 5-LO−/− mouse is equivalent to a 14 day callus in a normal mouse; that a 14 day 5-LO−/− callus is equivalent to a 21 day normal callus; and that a 1 month 5-LO−/− callus is equivalent to a 3 month normal callus (FIG. 3). Thus, loss of 5-LO function accelerates and/or enhances the regenerative and remodeling phases of fracture healing.

In one aspect of the invention, compounds that inhibit 5-lipoxygenase activity accelerate and/or enhance healing of a bone fracture or prevent bone resorption or promote bone formation provide important benefits to efforts at treating human disease. Compounds that inhibit 5-lipoxygenase activity can be used, e.g., in a method for treating bone fracture due to trauma, or due to osteoporosis or osteoarthritis, in a method for treating Paget's disease, in a method for treating other conditions such as bone transplants and diseases associated with increased bone fracture, and in methods that require bone formation such as spinal fusions, other bone and joint ankylosis procedures, bone or limb lengthening, augmentation of bone structure, incorporation of allograft, autograft, or synthetic bone material into treatment sites, bone growth into or around prosthetic devices, bone growth associated with dental procedures, and other similar procedures.

Several inhibitors of 5-lipoxygenase and/or FLAP and their dosing are known which are useful for practicing the methods of the invention. A FLAP inhibitor can be 3-[1-(4-chlorobenzyl)-3-t-butyl-thio-5-isopropylindol-2-yl]-2,2-dimethylpropanoic acid (MK886) or derivatives thereof; 3-(1-(4-chlorobenzyl)-3-(1-butyl-thio)-5-(quinolin-2-yl-methoxy)-indol-2-yl)-2,2-dimethyl propanoic acid) (MK-591) or derivatives thereof; and Amira Pharmaceuticals AM-103. A 5-LO inhibitor can be nordihydroguaiaretic acid (NDGA) or derivatives thereof; 2-(12-hydroxydodeca-5,10-diynyl)-3,5,6-trimethyl-1,4-benzoquinone (AA861) or derivatives thereof; or (N-(1-benzo[b]thien-2-ylethyl)-N-hydroxyurea) (Zileuton) or derivatives thereof. Derivatives include, e.g., pharmaceutically acceptable salts, prodrugs, etc., which also are useful as 5-lipoxygenase and/or FLAP inhibitors. Derivatives of exemplary compounds are intended to be within the scope of the claimed invention.

Other 5-lipoxygenase inhibitors for use in the invention include masoprocol, tenidap, flobufen, lonapalene, tagorizine, Abbott A-121798, Abbott A-76745, Abbott A-78773, Abbott A-79175, Abbott ABT 761, Dainippon AL-3264, Bayer Bay-x-1005, Biofor BF-389, bunaprolast, Cytomed CMI-392, Takeda CV-6504, enazadrem phosphate, Leo Denmark ETH-615, flezelastine hydrochloride, Merck Frosst L-663536, Merckle ML-3000, 3M Pharmaceuticals R-840, rilopirox, Schering Plough SCH-40120, tepoxalin, linazolast (TMK-688), Zeneca ZD-2138, Zeneca ZD-4407, Bristol-Myers Squibb BU-4601A, carbazomycin C, lagunamycin, Wellcome BW-70C, Ciba-Geigy CGS-26529, Warner-Lambert CI 1004, Warner-Lambert PD-136005, Warner-Lambert PD-145246, Elsai E-3040, Fujirebio F-1322, Fujisawa FR-110302, Merck Frosst L-699333, Merck Frosst L-739010, Lilly LY-269415, Lilly LY-178002, Hoechst Roussel P-8892, SmithKline Beecham SB-202235, American Home Products WAY-121520, American Home Products WAY-125007, Zeneca ZD-7717, Zeneca ZM-216800, Zeneca ZM-230487, 1,2-dihydro-n-(2-thiazolyl)-1-oxopyrrolo(3,2,1-kl)phenothiazine-1-carboxamide, Abbott A-65260, Abbott A-69412, Abbott-63162, American Home Products AHR-5333, Bayer Bay-q-1531, Boehringer Ingelheim BI-L-357, Boehringer Ingelheim BI-L-93BS, Boehringer Ingelheim BIL 226XX, Bristol-Myers Squibb BMY-30094, carbazomycin B, Wellcome BW-B218C, Chauvin CBS-1114, Ciba-Geigy CGS-21595, Ciba-Geigy CGS-22745, Ciba-Geigy CGS-23885, Ciba-Geigy CGS 24891, Ciba-Geigy CGS-8515, Chiesi CHF-1909, Warner-Lambert CI-986, Warner-Lambert CI-987, cirsiliol, docebenone, Eisai E-5110, Eisai E-6080, enofelast, epocarbazolin-A, eprovafen, evandamine, Fisons FPL 62064, Zeneca ICI-211965, Zeneca ICI-216800, Kyowa Hakko KF-8940, Merck & Co L-651392, Merck & Co L-651896, Merck & Co L-652343, Merck & Co L-656224, Merck & Co L-670630, Merck & Co L-674636, Lilly LY-233569, Merck & Co MK-591, Merck & Co L-655240, nitrosoxacin-A, Ono ONO-5349, Ono ONO-LP-219, Ono ONO-LP-269, Warner-Lambert PD-127443, Purdue Frederick PF-5901, Rhone-Poulenc Rorer Rev-5367, Rhone-Poulenc Rorer RG-5901-A, Rhone-Poulenc Rorer RG-6866, Roussel-Uclaf RU-46057, Searle SC-41661A, Searle SC-45662, Sandoz SDZ-210-610, SmithKline Beecham SK&F-104351, SmithKline Beecham SK&F-104493, SmithKline Beecham SK&F-105809, Synthelabo SL-81-0433, Teijin TEI-8005, Terumo TMK-777, Terumo TMK-781, Terumo TMK-789, Terumo TMK-919, Terumo TMK-992, Teikoku Hormone TZI-41127, American Home Products WAY-120739, American Home Products WY-47288, American Home Products WY-48252, American Home Products WY-50295, American Home Products WY-50295T, Yoshitomi Y-19432, 4-{3-[4-(2-methyl-1H-imidazol-1-yl)phenylthio]}phenyl-3,4,5,6-tetrahydro-2H-pyran-4-carboxamide, esculetin, phenidone and its derivatives, BI-L-239, 5,8,11-eicosatriynoic acid (ETI), 5,8,11,14-eicosatetraynoic acid (ETYA), cinnamyl-3,4-dihydroxy-alpha-cyanocinnamate, curcumin, esculeitin, gossypol, caffeic acid, baicalein, 7,7-dimethyleicosadrenoic acid (DEDA), Ly311727, bromoenol lactone, methyl arachidonyl fluorophosphonate, methyl y-linolenyl fluorophosphonate, oleyoxyethyl phosphorylcholine, AACOCF3, n-(p-amylcinnamoyl) anthranilic acid, mepacrine, quinacrine, atabrine, parabromophenacylbromide, aristolochic acid, corticosteroids, Glaxo SmithKline 480848, Glaxo SmithKline 659032, Glaxo SmithKline 677116, BMS-181162, MJ33, Millennium Pharmaceuticals MLN977, Dainippon TA-270, Glaxo SmithKline SB-210661, and Ranbaxy Laboratories Limited RBx7796. Derivatives include, e.g., pharmaceutically acceptable salts, prodrugs, etc. which also are useful as LTA4-H inhibitors. Derivatives of exemplary compounds are intended to be within the scope of the claimed invention.

More preferred 5-lipoxygenase inhibitors include masoprocol, tenidap, zileuton, flobufen, lonapalene, tagorizine, AA-861, Abbott A-121798, Abbott A-76745, Abbott A-78773, [(R)(+)N′-[[5-(4-fluorophenoxy)furan-2-yl]-1-methyl-2-propynyl]-N-hydroxyurea (Abbott A-79175),] Abbott A-79175, Abbott ABT 761, Dainippon AL-3264, Bayer Bay-x-1005, Biofor BF-389, bunaprolast, Cytomed CMI-392, Takeda CV-6504, Ciba-Geigy CGS-26529, enazadrem phosphate, Leo Denmark ETH-615, flezelastine hydrochloride, Merck Frosst L 663536, Merck Frosst L 699333, Merckle ML-3000, 3M Pharmaceuticals R-840, rilopirox, Schering Plough SCH 40120, tepoxalin, linazolast (TMK-688), Zeneca ZD-7717, Zeneca ZM-216800, Zeneca ZM-230487, Zeneca ZD-2138, Zeneca ZD-4407, American Home Products WY-50295, American Home Products WY-30295T, Millennium Pharmaceuticals MLN977, Dainippon TA-279, Glaxo SmithKline SB-210-661, Ranbaxy Laboratories Limited RBx7796, Merck MK-886, Merck MK-591, Amira Pharmaceuticals AM-103, and NDGA (nondihydroguaiaretic acid). Derivatives include, e.g., pharmaceutically acceptable salts, prodrugs, etc. which also are useful as LTA4-H inhibitors. Derivatives of exemplary compounds are intended to be within the scope of the claimed invention.

Even more preferred 5-lipoxygenase inhibitors include zileuton, AA-861, Abbott A-121798, Abbott A-76745, Abbott A-78773, Abbott A-79175, Abbott ABT 761, Ciba-Geigy CGS-26529, Biofor BF-389, Cytomed CMI-392, Leo Denmark ETH-615, Merck Frosst L 699333, Merckle ML-3000, 3M Pharmaceuticals R-840, linazolast (TMK-688), Zeneca ZD-7717, Zeneca ZM-216800, Zeneca ZM-230487, Zeneca ZD-2138, Zeneca ZD-4407, Millennium Pharmaceuticals MLN977, Merck MK-886, Merck MK-591, Amira Pharmaceuticals AM-103, American Home Products WY-50295, American Home Products WY-50295T, Dainippon TA-027, Glaxo SmithKline SB-210661, Ranbaxy Laboratories Limited RBx7796, and NDGA (nondihydroguaiaretic acid). Derivatives include, e.g., pharmaceutically acceptable salts, prodrugs, etc., which also are useful as LTA4-H inhibitors. Derivatives of exemplary compounds are intended to be within the scope of the claimed invention.

Exemplary dose ranges of 5-LO and FLAP inhibitors in humans include, e.g., zileuton dose of 600 mg four times per day; ABT-761/VIA-2291 dose of 100 mg per day; CV6504 dose of 100 mg three times per day; MLN977 dose range of 200 to 600 mg per day; MK-886 dose range of 250 to 500 mg per day; and MK-591 dose ranges of 50 to 250 mg per day and 250 mg twice per day.

Several inhibitors of LTA4-H and their dosing are known which are useful for practicing the methods of the invention. A LTA4-H inhibitor can be Johnson & Johnson JNJ-26993135 (1-[4-(benzothiazol-2-yloxy)-benzyl]-piperidine-4-carboxylic acid), Santen Pharmaceutical SA-6541 (5-(4-dimethylaminobenzyl)-N-[(2S)-3-mercapto-2-methylpropionyl]-L-cysteine), Santen Pharmaceutical SA-9499 (S-(4-Cyclohexylbenzyl)-N-[2(S)-methyl-3-sulfanylpropionyl]-L-cysteine), Pfizer/Searle SC-22716 (1-[2-(4-Phenylphenoxy)ethyl]pyrrolidine), Pfizer/Searle SC-56938 (ethyl-1-[2-[4-(phenylmethyl)phenoxy]ethyl]-4-piperidine-carboxylate), captopril, bestatin, DeCODE DG-051, 4-phenylchalcone oxide, leukotriene A3, leucine thiol, 1,10-phenanthroline, 8-hydrxyquinoline-5-sulfonic acid, kelatorphan, cyclosporine A, Johnson & Johnson JNJ-27265732, SC-57461 (N-methyl-N-[3-[4-(phenylmethyl)-phenoxy]propyl]-beta-alanine), SC-57461A (3-[methyl[3-[4-phenylmethyl)phenoxy]propyl]amino]propanoic acid HCl), Rhône-Poulenc Rorer RP-64966, 8(S)-amino-2(R)-methyl-7-oxononanoic acid, 3-(4-benzyloxyphenyl)-2-(R)-amino-1-propanethiol, LY-293111 [2-[2-propyl-3-[3-[2-ethyl-4-(4-fluorophenyl)-5-hydroxyphenoxy]-propoxy]-phenoxy]benzoic acid], ONO Pharmaceutical ONO-4057, (E)-7-(2-diethylcarbamoyl-1-methylvinyl)benzo[b]furan, LTB019, U-75302 (6-(6-(3R-hydroxy-1E,5Z-undecadien-1-yl)-2-pyridinyl)-1,5S-hexanediol), Resolvin E1, BIIL284, LY-255283 [1-(5-ethyl-2-hydroxy-4-(6-methyl-6-(1H-tetrazol-5-yl)-heptoxy+++)-phenyl)ethanone], CP-105,696 [(+)-1-(3S,4R)-[3-(4-phenyl-benzyl)-4-hydroxy-chroman-7-yl]-cyclopentane carboxylic acid], SB-209247 [(E)-3-[6-[[(2,6-dichlorophenyl)-thio]methyl]-3-(2-phenylethoxy)-2-pyridinyl]-2-propenoic acid], CP-195,543 [(+)-2-(3-benzyl-4-hydroxy-chroman-7-yl)-4-trifluoromethyl-benzoic acid], ZK158252, CGS25019C, LY-223982, SB-201146, SB-201993 [(E)-3-[[[[6-(2-carboxyethenyl)-5-[[8-(4-methoxyphenyl)octyl]oxy]-2-pyridinyl]methyl]thio]methyl]benzoic acid], RO-0254094 (2-[(5-carboxypentyl)-6-[6-[3,4-dihidro-4-oxo-8-propyl-2H-1-benzopyran-7-yl)hexyl]benzenepropanoic acid), SC-41930 [7-[3-(4-acetyl-3-methoxy-2-propylphenoxy)propoxyl]-3,4-dihydro-8-propyl-2H-1-benzopyran-2-carboxylic acid], PH-163 [(1S*,3S*)-1-hydroxy-3-[(3R*S,E)-3-hydroxy-7-phenyl-1-hepten-1-yl]-1-cyclohexane acetate], SC-53228 [(+)-(S)-7-[3-(2-cyclopropyl-methyl)-3-methoxy-4-[(methylamino) carbonyl]phenoxy]propoxy]-3,4-dihydro-8-propyl-2H-1-benzopyran-2-propanoic acid], SM-15178, SC-51146, SC-53229, and SC-45694 [7-[4-(1-hydroxy-3Z-nonenyl)phenyl]-5S-hydroxy-6Z-hept enoic acid lithium salt]. Derivatives include, e.g., pharmaceutically acceptable salts, prodrugs, etc. which also are useful as LTA4-H inhibitors. Derivatives of exemplary compounds are intended to be within the scope of the claimed invention.

Dose ranges of the LTA4-H inhibitors can include, e.g.: SC-57461A (3-[methyl[3-[4-phenylmethyl)phenoxy]propyl]amino]propanoic acid HCl), dose range 0.5-10 mg/kg; SC-56938, dose range 0.5-10 mg/kg; captopril, 25-150 mg, two or three times a day for humans.

Several antagonists of Leukotriene B4 receptor and their dosing are known which are useful for practicing the methods of the invention. A Leukotriene B4 receptor antagonist can be Eli Lilly LY-255283 [1-(5-ethyl-2-hydroxy-4-(6-methyl-6-(1H-tetrazol-5-yl)-heptyloxy)phenyl)ethanone] or derivatives thereof, Eli Lilly LY-223982 or derivatives thereof, Eli Lilly LY-293111 [2-(2-propyl-3-(3-(2-ethyl-4-(4-fluorophenyl)-5-hydroxyphenoxy)propoxy)phenoxy)benzoic acid] or derivatives thereof, GSK/SmithKline Beecham SB-201146 [3-(6-(((3-aminophenyl)sulfinyl)methyl)-3-(8-(4-methoxyphenyl) octyl)oxy)pyridin-2-yl)acrylic acid lithium salt] or derivatives thereof, GSK/SmithKline Beecham SB-225002 or derivatives thereof, Pfizer/Searle SC-41930 [7-93-(4-acetyl-3-methoxy-2-propylphenoxy)propoxyl)-3,4-dihydro-8-propyl-2H-1-benzopyran-2-carboxylic acid] or derivatives thereof, Pfizer/Searle SC-53228 [(+)-(S)-7-(3-(2-(cyclopropylmethyl)-3-methoxy-4-((methylamino)carbonyl)phenoxy)propoxy)-3,4-dihydro-8-propyl-2H-1-benzopyran-2-propanoic acid] or derivatives thereof, Boehringer Ingelheim BIIL284 [N-(ethoxycarbonyl)-4-(3-(4-(1-(4-hydroxyphenyl)-1-methylethyl)-phenoxy-methyl)benzyloxy)benzenecarboximidamide] or derivatives thereof, Pfizer CP-105696 [(+)-1-(3S,4R)-[3-(4-phenylbenzyl)-4-hydroxychroman-7-yl]cyclopentane carboxylic acid] or derivatives thereof, Ono ONO-4057 i.e., ONO-LB-457 [(5-[2-(2-Carboxyethyl)-3-[6-(4-methoxyphenyl)-5E-hexenyl]oxyphenoxy]valeric acid] or derivatives thereof, Pfizer/Searle SC-50605 [7-[3-[2(cyclopropylmethyl)-3-methoxy-4-(4-thiazolyl)phenoxy]propoxy]-3,4-dihydro-8-propyl-2H-1-benzopyran-2-carboxylic acid] or derivatives thereof, FPL-55712 [7-[3-(4-Acetyl-3-hydroxy-2-propylphenoxy)-2-hydroxypropoxy]-4-oxo-8-propyl-4H-1-benzopyran-2-carboxylic acid sodium salt] or derivatives thereof, Schering ZK-158252 [5-[2-[5-Hydroxy-5-[1-(3-phenyl-2-propynyl)cyclobutyl]-1,3-pentadienyl]cyclohexylidene]-pentanoic acid] or derivatives thereof, Pfizer CP-195543 [(+)-2-(3-benzyl-4-hydroxy-chroman-7-yl)-4-trifluoromethyl-benzoic acid] or derivatives thereof, Rhone-Poulenc Rorer RG-14893 [4-[2-[Methyl(2-phenethyl)amino]-2-oxoethyl]-8-(phenylmethoxy)-2-naphthalenecarboxylic acid] or derivatives thereof, GSK/SmithKline Beecham SB-209247 [(E)-3-[6-[[(2,6-dichlorophenyl)-thio]methyl]-3-(2-phenylethoxy)-2-pyridinyl]-2-propenoic acid] or derivatives thereof, CGS25019C or derivatives thereof, Rhone-Poulenc RP-69698 [5,6-bis-4,5-(4-meth-oxyphenyl)-2-oxazolylhexyltetrazol] or derivatives thereof, Pfizer/Searle SC-51146 (7-[3-[2(cyclopropylmethyl)-3-methoxy-4-[(methylamino)carbonyl]phenoxy]propoxy]-3,4-dihydro-8-propyl-2H-1-benzopyran-2-propanoic acid) or derivatives thereof, and Upjohn U-75302 6-(6-(3-hydroxy-1E,5Z-undecadien-1-yl)-2-pyridinyl)-1,5-hexa nediol or derivatives thereof. Derivatives include, e.g., pharmaceutically acceptable salts, prodrugs, etc. which also are useful as Leukotriene B4 receptor antagonists. Derivatives of exemplary compounds are intended to be within the scope of the claimed invention.

Dose ranges of the Leukotriene B4 receptor antagonists can include, e.g., BIIL 284, dose 25 to 75 mg per day for humans [see Díaz-González, et al. Clinical trial of a leucotriene B4 receptor antagonist, BIIL 284, in patients with rheumatoid arthritis. Annals of the Rheumatic Diseases 66:628-632 (2007)]; LY293111, dose 200-800 mg per day for humans [see Schwartz, et al. Phase I and Pharmacokinetic Study of LY293111, an Orally Bioavailable LTB4 Receptor Antagonist, in Patients With Advanced Solid Tumors. Journal of Clinical Oncology 23:5365-5373 (2005)]; SC-41930, dose range 0.5-10 mg/kg in humans; SC-50605, dose range, 0.1-5 mg/kg in humans; SC-53228, dose range 2-20 mg/kg in humans.

Many of the inhibitors and antagonists as well as others have been described in the art, e.g., Rao et al., Anti-Inflammatory Activity of a Potent, Selective Leukotriene A4 Hydrolase Inhibitor in Comparison with the 5-LO Inhibitor Zileuton, The Journal of Pharmacology and Experimental Therapeutics; 321: 1154-1160 (2007), the disclosure of which is hereby incorporated by reference; Penning et al., Inhibitors of LTA4H as Potential Anti-Inflammatory Agents, Current Pharmaceutical Design; 7: 163-179 (2001), the disclosure of which is hereby incorporated by reference; and Daines et al., J. Med. Chem., 39 (19), 3837-3841, 1996, the disclosure of which is hereby incorporated by reference.

In another aspect, the invention comprises a 5-LO inhibitor, FLAP inhibitor, LTA4-H inhibitor, LTBR receptor antagonist, and/or a LTBR2 receptor antagonist and a COX inhibitor and its use. Preferably, the COX inhibitor is a selective COX-1 inhibitor, i.e., that it inhibits the activity of COX-1 more than it inhibits the activity of COX-2. The use of a 5-LO inhibitor, FLAP inhibitor, LTA4-H inhibitor, LTBR receptor antagonist, and/or a LTBR2 receptor antagonist and a COX inhibitor is intended to embrace administration of each inhibitor in a sequential manner in a regimen that will provide beneficial effects of the drug combination, the co-administration of the inhibitors in a substantially simultaneous manner, such as in a single capsule having a fixed ratio of these active agents, or in multiple, separate capsules for each agent, as well as a single compound that inhibits multiple enzymes.

The COX inhibitor can be selected from the group consisting of celecoxib; rofecoxib; meloxicam; piroxicam; valdecoxib, parecoxib, etoricoxib, CS-502, JTE-522; L-745,337; FR122047; NS398; from non-selective NSAIDs that would include aspirin, ibuprofen, indomethacin CAY10404, diclofenac, ketoprofen, naproxen, ketorolac, phenylbutazone, tolfenamic acid, sulindac, and others, or from steroids or corticosteroids. Compounds which selectively inhibit cyclooxygenase-2 have been described in U.S. Pat. Nos. 5,380,738, 5,344,991, 5,393,790, 5,466,823, 5,434,178, 5,474,995, 5,510,368 and WO documents WO96/06840, WO96/03388, WO96/03387, WO95/15316, WO94/15932, WO94/27980, WO95/00501, WO94/13635, WO94/20480, and WO94/26731, and are otherwise known to those of skill in the art.

Selective COX-1 inhibitors are known in the art. The following is a list of preferred COX-1 selective NSAIDs: SC-560 [Smith et al., Proceedings of the National Academy of Sciences of the United States of America 95:13313-8 (1998)], FR122047 [Dohi et al., European Journal of Pharmacology 243:179-84 (1993)], Valeroyl salicylate, and Aspirin. Aspirin is an irreversible cyclooxygenase inhibitor that is rapidly inactivated in vivo. While aspirin can inhibit COX-1 and COX-2, prior treatment with aspirin can inactivate all pre-existing COX-1 before or during expression of COX-2. Thus any new COX-2 that is expressed is active but all “older” COX-1 or COX-2 is inactivated.

The following is a list of NSAIDs that preferentially inhibit COX-1 versus COX-2: Dexketoprofene, Keterolac, Flurbiprofen, Suprofen. See also [Warner et al., Nonsteroid drug selectivities for cyclo-oxygenase-1 rather than cyclo-oxygenase-2 are associated with human gastrointestinal toxicity: a full in vitro analysis. Proceedings of the National Academy of Sciences of the United States of America 96:7563-8 (1999)].

In another embodiment, the invention comprises a 5-LO inhibitor, FLAP inhibitor, LTA4-H inhibitor, LTBR receptor antagonist, and/or a LTBR2 receptor antagonist and a COX-2 activator and its use. COX-2 activators also are known in the art. See [Tanabe and Tohnai, Cyclooxygenase isozymes and their gene structures and expression. Prostaglandins & other Lipid Mediators 68-69:95-114 (2002)] for review article concerning regulation of COX-2 gene expression and as a reference for those compounds or treatments listed below without a reference. Preferred COX-2 activators include ultrasound therapy [Sena et al., Early gene response to low-intensity pulsed ultrasound in rat osteoblastic cells. Ultrasound in Medicine & Biology 31:703-8 (2005)], pulsed electromagnetic fields (PEMF) [Lohmann et al., Pulsed electromagnetic fields affect phenotype and connexin 43 protein expression in MLO-Y4 osteocyte-like cells and ROS 17/2.8 osteoblast-like cells. Journal of Orthopaedic Research 21:326-34 (2003)], BMP-2 [Chikazu et al., Bone morphogenetic protein 2 induces cyclo-oxygenase 2 in osteoblasts via a Cbfa1 binding site: role in effects of bone morphogenetic protein 2 in vitro and in vivo. Journal of Bone and Mineral Research 17:1430-40 (2002)], BMP-7, PDGF, FGF, recombinant forms of BMP-2, BMP-7, PDGF and FGF, and PTH and its analogs (PTHrP and teriparatide) [Maciel et al., Induction of cyclooxygenase-2 by parathyroid hormone in human osteoblasts in culture. Journal of Rheumatology 24:2429-35 (1997)]. Other COX-2 activators include prostaglandins and prostaglandin receptor agonists [Rosch et al., Prostaglandin E2 induces cyclooxygenase-2 expression in human non-pigmented ciliary epithelial cells through activation of p38 and p42/44 mitogen-activated protein kinases. Biochemical and Biophysical Research Communications 338:1171-8 (2005)], PDGF (platelet derived growth factor), IL-1alpha (interleukin 1 alpha), IL-1beta, TNF-alpha (tumor necrosis factor alpha), FGF (fibroblast growth factor), TGF-beta (transforming growth factor beta), TGF-alpha, EGF (epidermal growth factor), and TPA (tetradecanoyl phorbol acetate) and recombinant forms of these activators.

In addition, the invention comprises a combination comprising a therapeutically-effective amount of a 5-lipoxygenase inhibitor, FLAP inhibitor, LTA4-H inhibitor, LTBR receptor antagonist, and/or a LTBR2 receptor antagonist and a cyclooxygenase-2 inhibitor, such as, e.g., licofelone, Dupont Dup 697, Taisho NS-398, meloxicam, flosulide, Glaxo SmithKline 406381, Glaxo SmithKline 644784, or tepoxalin.

The modulation of bone metabolism by the methods of the invention can be determined by examination of bone strength and mass after administration compared to a control subject. Such examination can be performed in situ by using imaging techniques (e.g., X-ray, nuclear magnetic resonance imaging, X-ray tomography, ultrasound, and sound conduction) or stress testing, or ex vivo by standard histological, radiographic, mechanical, or biochemical methods. Modulation of bone density and/or bone mass can be assessed by changes in one or more parameters such as bone mineral density, bone strength, trabecular number, bone size, and bone tissue connectivity. Several methods for determining bone mineral density (BMD) are known in the art. For example, BMD measurements may be done using, e.g., dual energy x-ray absorptiometry or quantitative computed tomography, and the like. Similarly, increased bone formation can be determined using methods well known in the art. For example, dynamic measurements of bone formation rate (BFR) can be performed on tetracycline labeled cancellous bone from the lumbar spine and distal femur metaphysis using quantitative digitized morphometry [Liang et al., Bone anabolic effects of basic fibroblast growth factor in ovariectomized rats. Endocrinology 140: 5780-5788 (1999)]. Alternatively, bone formation markers, such as alkaline phosphatase activity, serum collagen peptide levels, or serum osteocalcin levels can be assessed to indirectly determine whether increased bone formation has occurred [Looker et al., Clinical use of biochemical markers of bone remodeling: current status and future directions. Osteoporosis International 11: 467-480 (2000)]. Compounds that modulate an arachidonic acid metabolic or signaling pathway can be tested for their ability to accelerate or enhance fracture healing and/or bone formation, promote bone formation, and prevent bone loss. This can be tested in a variety of animal models well known to one skilled in the art such as animal fracture models, animal osteotomy models, animal skull trephine defect models, animal bone defect models, various animals segmental defect models and bone lengthening models, ovariectomy induced bone loss models, and the like. The utility of these animal models is well established and is supported by a wide range of different observations. For example, BMP2 studies in animals including rats demonstrated that BMP2 stimulates osteogenesis and rhBMP2 is now used clinically in humans for bone repair applications (tradename INFUSE). There are hundreds of papers about the use of rhBMP in animals and tens of papers about humans. NSAIDs inhibit fracture repair in rats [Simon et al., Cyclooxygenase 2 function is essential for bone fracture healing. Journal of Bone and Mineral Research 17:963-76 (2002)] and NSAID use has been correlated to poor fracture healing in humans [Burd et al., Heterotopic ossification prophylaxis with indomethacin increases the risk of long-bone nonunion. Journal of Bone and Joint Surgery (British) 85B:700-5 (2003)]. Studies cited in Rubin et al. [The use of low-intensity ultrasound to accelerate the healing of fractures. Journal of Bone and Joint Surgery (American) 83A:259-270 (2001)] indicate that ultrasound treatment accelerates fracture repair in rats [Azuma, et al., Low-intensity pulsed ultrasound accelerates rat femoral fracture healing by acting on the various cellular reactions in the fracture callus. Journal of Bone and Mineral Research 16: 671-80 (2001)] and in humans. FDA guidelines for osteoporosis therapies indicate that preclinical studies require use of 2 species and that one must be an ovariectomized rat model. Thus methods that alter bone formation in animal models, including rats, can be measured and are considered to be predictive of the effects those methods will have in humans and other mammals.

Modulation of bone metabolism by the methods of the invention can be determined in vitro by examining the proliferation, survival, and differentiation of osteoblasts and/or chondrocytes following treatment that alters arachidonic acid metabolism as compared to mock treated cells. Treatment of cells or organ explants such as newborn rodent calvaria or phalanges can be with compounds that modulate an arachidonic acid metabolic or signaling pathway, alter cyclooxygenase activity, affect leukotriene or prostaglandin receptor function, and the like as set forth in this application. Additional treatment methods can include use of antisense nucleic acids, interfering RNAs, other nucleic acid or proteins, and the like. Osteoblast or chondrocyte proliferation and survival can be measured by a number of techniques well known to one skilled in the art such as cell counting, incorporation of radiolabeled thymidine or bromodeoxyuridine into replicating DNA, trypan blue exclusion, and terminal deoxynucleotidyl transferase end labeling of DNA within cells undergoing apoptosis. Differentiation of osteoblasts and/or chondrocytes can be measured by a number of techniques well known to one skilled in the art and would include formation of mineralized nodules stained by the method of von Kossa or with alizarin red to ascertain osteoblast or chondrocyte culture mineralization, alcian blue staining of chondrocytes to measure elaboration of proteoglycan matrix, gene expression analyses to measure markers of osteoblast and chondrocyte differentiation such as Type I, Type II, and Type X collagen, osteocalcin, and aggrecan using protein or nucleic acid based assay methods, measurement of alkaline phosphatase activity, and measurement of RANKL, OPG, VEGF, bone morphogenetic protein, and other growth factors by quantitative methods such as enzyme-linked immuno assays (EIA).

### 5-Lipoxygenase-Activating Protein (FLAP)

FLAP is an 18-kD membrane-bound polypeptide which specifically binds arachidonic acid and activates 5-LO by acting as an arachidonic acid transfer protein. The FLAP gene spans greater than 31 kb and consists of five small exons and four large introns (GenBank 182657, Genbank M60470 for exon 1, Genbank M63259 for exon 2, Genbank M63260 for exon 3, Genbank M63261 for exon 4, and Genbank M6322 for exon 5).

The nuclear envelope is the intracellular site at which 5-LO and FLAP act to metabolize arachidonic acid, and ionophore activation of neutrophils and monocytes results in the translocation of 5-LO from a nonsedimentable location to the nuclear envelope. Inhibitors of FLAP function prevent translocation of 5-LO from cytosol to the membrane and inhibit 5-LO activation. Thus, FLAP inhibitors are anti-inflammatory drug candidates.

Leukotriene synthesis is reduced by drugs that inhibit FLAP (MK886) or in mice lacking FLAP. Thus, in one aspect of the invention, FLAP inhibitors such as AM-103, BAYx 1005, MK-886, and MK-0591, are used in methods that modulate an arachidonic acid metabolic or signaling pathway thereby accelerating and/or enhancing fracture healing and bone formation.

### Leukotriene A₄ Hydrolase (LTA4-H)

LTA4-His an approximately 69 kDa protein of 610 amino acids. The human LTA4-H gene is located on chromosome 12 (12q22), is approximately 35,000 bp with 19 exons. Precursor mRNA from the LTA4-H gene is known to undergo alternative splicing that can produce multiple LTA4-H isoforms. The principle catalytic activities of LTA4-H are an aminopeptidase activity and an epoxide hydrolase activity that converts leukotriene A₄ into leukotriene B₄. Thus, LTA4-H activity is essential for the conversion of arachidonic acid into LTB4. In this biosynthetic pathway, arachidonic acid is first converted into LTA4 by 5-lipoxygenase in conjunction with its essential co-factor FLAP (five lipoxygenase activating protein). LTA4 is an intermediary metabolite and has no know biological function in and of itself. In the next step of the biosynthetic pathway, LTA4 is converted into LTB4 by LTA4-H. LTB4 is a biological active metabolite of arachidonic acid with well described biological activities that include the promotion of inflammation. Thus methods that inhibit the activity of LTA4-H can prevent or reduce the synthesis of LTB4 and thereby alter biological responses. Thus, in one aspect of this invention, LTA4-H inhibitors, such as SC-22716, SC-57461A, JNJ-26993135, SC-56938, or DG-051, or compositions that reduce the expression of LTA4-H, such as siRNAs or antisense molecules that target the LTA4-H mRNA, are used in methods that modulate an arachidonic acid metabolic or signaling pathway thereby accelerating and/or enhancing fracture healing and bone formation.

### Assays for Identifying Novel LTA4H Inhibitors

In vitro assay for LTA4-H activity [see Rao et al., Anti-inflammatory activity of a potent, selective leukotriene A₄ hydrolase inhibitor in comparison with the 5-lipoxygenase inhibitor zileuton. Journal of Pharmacology and Experimental Therapeutics 321:1154-1160 (2007)].

Recombinant human LTA4-H hydrolase (rhLTA4-H) is purchased from commercial sources or is prepared using recombinant baculovirus-infected insect cells using standard methods well known to one skilled in the art. rhLTA4-His diluted in assay buffer (0.1 M potassium phosphate, pH 7.4 with 5 mg/ml fatty-acid free bovine serum albumin) and the test compound (dissolved in an aqueous or organic solvent) is added (between 0.01 and 0.2% of the volume). The rhLTA4 and test compound are allowed to interact for 5-20 minutes at 18-37° C. Typically this step is performed in a volume of 50 μl (range 10-200 ul). An additional 3 volumes of assay buffer is added, typically 150 ul. The free acid form of LTA4 is added to a final concentration of 0.13 μM (40 ng/ml; range 0.1-0.15 μM) in volume not exceeding 15% of the final reaction volume, typically 25 ul of LTA4 solution into 200 ul of reaction mix. rhLTA4-H catalysis is allowed to occur for 10-30 minutes at 18-37° C. The reaction is stopped by a dilution with 0.1 M potassium phosphate pH 7.4 buffer and the amount of LTB4 formed in the reaction is measured using commercially available enzyme-linked immunoassay kits (LTB4 EIA kit; Caymen Chemical) or by other means well know to one skilled in the art. Inhibition of rhLTA4-His detected as a decreased amount of LTB4 synthesis as compared to a control reaction performed identically but without addition of any test compound.

### In Vitro Assay for LTA4-H Activity Using Whole Blood.

Whole blood is collected from a mammal (mouse, rat, rabbit, human) and treated with heparin to prevent coagulation. The blood is diluted with RPMI media (1:1 to 1:15 blood to media; typically 1:2) and 200 ul aliquots of the diluted blood are treated with test compounds dissolved in an appropriate solvent. Preferably the test compound is administered in volume of (0.1% volume, range 0.05 to 0.25%). The diluted blood and test compound are incubated at 18-37° C. for 5-30 minutes, preferable for 15 minutes at 37° C. The calcium ionophore A23187 is added to 20 μg/ml (range 5-50 μg/ml) and the reaction mixture is incubated 18-37° C. for an additional 5-60 minutes, preferably 30 minutes at 37° C. The reaction is terminated by centrifugation and collecting the supernatant. LTB4 is measured in the supernatant using an LTB4 EIA kit or other methods well known to one skilled in the art. LTA4-H inhibition is detected as a decreased LTB4 synthesis.

In vitro assay for LTA4-H Aminopeptidase activity [see Rao et al., Anti-inflammatory activity of a potent, selective leukotriene A₄ hydrolase inhibitor in comparison with the 5-lipoxygenase inhibitor zileuton. Journal of Pharmacology and Experimental Therapeutics 321:1154-1160 (2007)]. rhLTA4-H (250-500 ng) is incubated for 15 minutes at 18-37° C. in assay buffer (50 mM Tris-Cl, pH 8.0 with 100 mM potassium chloride) with the test compound (neat or diluted in an appropriate aqueous or organic solvent). Preferably the volume of test compound solution added is less than 0.5% of the volume. After incubation to allow the test compound and LTA4-H to interact, an equal volume of substrate solution is added (2 mM L-alanine-4-nitro-anilide hydrochloride in 50 mM Tris-Cl, pH 8 with 100 mM potassium chloride). The reaction is allowed to proceed at 18-37° C. for 1-60 minutes and LTA4-H aminopeptidase activity is measured as an increase in absorbance of the reaction mixture at 405 nm. Inhibition of LTA4-H aminopeptidase activity is detected as reduced reaction mixture absorbance as compared to a control reaction that contained no test compound. Typically, the reactions are performed in volumes of 50-200

In vivo assay for LTA4-H Inhibition using an ex vivo whole blood assay [see Kachur et al.; Pharmacological characterization of SC-57461A (3-[methyl[3-[4-(phenylmethyl)phenoxy]propyl]amino]propanoic acid HCl), a potent and selective inhibitor of leukotriene A(4) hydrolase II: in vivo studies. Journal of Pharmacology and Experimental Therapuetics 300:583-587 (2002)]. A mammalian subject (mouse, rat, human) is treated with the test compound by oral administration, intraperitoneal, intravenous, intramuscular, or subcutaneous, or other method of substance delivery well known to one skilled in the art. The test compound may be administered neat or with appropriate excipients well known to one skilled in the art. Following test compound administration, blood is withdrawn from the subject between 1 minute and 48 hours after administration but more preferably 1, 2, 4, 8, 12, and 24 hours after test compound administration. The blood is collected into heparinized tubes to prevent coagulation. Using a microtiter plate or small test tubes, 100 μl of the blood is diluted with 100 μl of RPMI media. The calcium ionophore A23187 is added to a final concentration of 20 μg/ml to initiate LTB4 synthesis and the mixture is incubated at 18-37° C. for 5-60 minutes, typically at 37° C. for 30 minutes. The diluted blood reaction mix is subjected to centrifugation and the supernatant is assayed for synthesis of LTB4 using an LTB4 EIA kit or other suitable method well known to one skilled in the art.

Additional in vitro and in vivo assays of LTA4-H activity are described in the literature and are well known to one skilled in the art. Such assays would include measurement of arachidonic acid-induced ear inflammation, measurement of myeloperoxidase activity in tissue samples, peritoneal eicosanoid formation, and the like.

### Leukotriene B4 Receptors

To assert its biological function, LTB4 interacts with one of 2 G-protein coupled cell surface receptors called LTBR (also called LTB4R or BLT1, Entrez Gene ID: 1241) and LTBR2 (also called LTB4R2 or BLT2, Entrez Gene ID: 27141). The LTBR gene is located on chromosome 14 (14q11.2-14q12) and encodes a protein of approximately 38 kDa and 352 amino acids. The LTBR2 gene is located on chromosome 14 and encodes a protein of 42 kDa and 389 amino acids. Tager and Luster [BLT1 and BLT2: the leukotriene B4 receptors. Prostaglandins, Leukotrienes and Essential Fatty Acids 69:123-134 (2003)] disclose that LTBR is a high affinity receptor for LTB4 and that LTBR2 is a low affinity receptor for LTB4. When LTB4 interacts with LTBR and LTBR2, it induces changes in intracellular calcium levels or can induce synthesis of inositol phosphates. These second messengers (Ca²⁺ and inositol phosphates) can lead to subsequent changes in cellular gene expression. Thus, in one aspect of this invention, LTBR or LTBR2 antagonists, such as LY-293111, ONO-4057, BIIL 284, SC-53228, SC-41930, LY-255283, or CP-195,543; or compositions that reduce the expression of LTBR or LTBR2 mRNA levels using nucleic acids such as siRNAs or antisense molecules specifically targeted to the LTBR mRNA or LTBR2 mRNA are used in methods that modulate an arachidonic acid metabolic or signaling pathway, thereby accelerating and/or enhancing fracture healing and bone formation.

### Leukotriene B4 Receptor Antagonist Assays

In vitro assay to identify LTBR or LTBR2 antagonists This assay measures the increase in intracellular calcium caused by activation of LTBR (BLT1) or LTBR2 (BLT2). A cell line is established that expresses human or another mammalian version of LTBR or LTBR2 using methods well known to one skilled in the art. For instance, a cDNA clone of LTBR or LTBR2 could be cloned into a mammalian expression vector that directs expression of the LTBR or LTBR2 cDNA and that enables selection of a cell line stably expressing exogenous LTBR or LTBR2. Mammalian expression vectors that perform this function are well known in the art and include vectors such as pcDNA3 (Invitrogen) that uses the cytomeglavirus early promoter to direct exogenous gene expression and that also expresses a gene for neomycin resistance that enables selection of stably expressing cell lines using antibiotic selection (G418 selection). A subclone made by inserting the LTBR or LTBR2 cDNA into pcDNA3 is transfected into a mammalian cell line, such as the 293 human kidney epithelial cell line using methods well known in the art such as calcium phosphate precipitation. Cell lines are identified that stably express LTBR or LTBR2. The 293-LTBR or 293-LTBR2 cell lines are cultured in vitro in microplates to obtain sufficient cell numbers per well of the microplate. The effects of LTB4 excitation on the flux of intracellular calcium is measured for each well of the microplate in the presence of vehicle or different concentration of test compound. Intracellular calcium flux is measured using commercially available reagents (FLIPR Calcium Assay Kit, Molecular Devices) and a fluorescence microplate reader. For instance 50,000 293-LTBR or 293-LTBR2 cells are cultured overnight in a volume of 100 μl of media (DMEM with 10% fetal bovine serum) in each well of a 96-well microtiter dish. The next day, the cells are treated by addition of 100 μl per well of calcium chloride (0.5-2 mM), 2 ug/ml fura-2-acetoxymethyl ester (fura-2/AM), 2.5 mM probenecid in Hank's balanced salt solution with HEPES buffer. Then 50 μl of a test compound solution or the diluent is added per well and the microplates are incubated at 37° C. for 1 hour. To each well of the microtiter dish an additional 50 μl of HBSS containing freshly diluted LTB4 is added and the change in fluorescence is measured using the fluorescence microplate reader. For 293-LTBR cells, final LTB4 concentrations of 1-100 μM are sufficient while for 293-LTBR2 cells, final LTB4 concentration of 1-10 nM are sufficient. If the test compound has LTBR or LTBR2 antagonist activity, it will prevent LTB4 from inducing intracellular calcium flux as measured by change in peak fluorescence between control and test compound values. Use of this assay procedure to measure LTBR or LTBR2 activity or test for the LTBR/LTBR2 antagonistic activity of different compounds can be found in Tarlowe et al. [Inflammatory chemoreceptor cross-talk suppresses leukotriene B₄ receptor 1-mediated neutrophil calcium mobilization and chemotaxis after trauma. Journal of Immunology 171: 2066-2073 (2003)], Peres et al. [Specific leukotriene receptors couple to distinct G proteins to effect stimulation of alveolar macrophage host defenese functions. Journal of Immunology 179:5454-5461 (2007)], and Huang et al. [Leukotriene B₄ strongly increases monocyte chemoattractant protein-1 in human monocytes. Arterioscler. Thromb. Vasc. Biol. 24:1783-1788 (2004)].

In vitro assay for LTB4 receptor activity or antagonism Huang et al. [Leukotriene B₄ strongly increases monocyte chemoattractant protein-1 in human monocytes. Arterioscler. Thromb. Vasc. Biol. 24:1783-1788 (2004)] teaches that LTB4 dramatically increases the expression monocyte chemoattractant protein-1 (MCP-1) at the mRNA and protein level. Thus LTB4 activity can be assayed indirectly by treating human monocytes in vitro with LTB4 and measuring the increase in MCP-1 protein, MCP-1 mRNA, or MCP-1 gene expression. Similarly, an antagonist of LTBR or LTBR2 would suppress LTB4 induced increases in MCP-1 protein, mRNA, or gene expression levels. One skilled in the art could reduce this to an assay procedure to measure LTB4 receptor antagonist activity. For instance, Xing and Remick [Promoter elements responsible for antioxidant regulation of MCP-1 gene expression. Antioxid Redox Signal. 9:1979-1989 (2007)] teaches that the promoter region of the human MCP-1 gene can be used to direct luciferase expression the activity of which can be easily measured by one of ordinary skill in the art. By transfecting primary human monocytes or established human monocyte cell lines, such as the Mono-Mac 6 cell line, with an MCP-1 promoter-luciferase construct, luciferase expression could be controlled by treatment with LTB4. Thus by pre-treating monocyte cells transfected with the MCP-1 promoter-luciferase construct with compound to be tested for LTB4 receptor antagonist activity, and subsequent treatment with LTB4, an inhibition in luciferase activity as compared to control treated cells would indicate that the test compound has LTBR or LTBR2 antagonist activity.

Other assays for LTB4 receptor activity or antagonism that measure increased IP3 or neutrophil influx as myleoperoxidase activity are well known in the art.

### Antisense Treatment

The term “antisense nucleic acid” is intended to refer to an oligonucleotide complementary to the base sequences of 5-LO, FLAP, LTA4-H, LTBR, and/or LTBR2-encoding DNA and RNA or those that encode other proteins in an arachidonic acid metabolic or signaling pathway. Antisense oligonucleotides can be modified or unmodified RNA, DNA, or mixed polymer oligonucleotides, and, when introduced into a target cell, specifically bind to their target nucleic acid and interfere with transcription, RNA processing, transport and/or translation. Targeting double-stranded (ds) DNA with oligonucleotide leads to triple-helix formation; targeting RNA will lead to double-helix formation.

Antisense constructs can be designed to bind to the promoter and other control regions, exons, introns or even exon-intron boundaries of a gene. Antisense RNA constructs, or DNA encoding such antisense RNAs, can be employed to inhibit gene transcription or translation or both within a host cell, either in vitro or in vivo, such as within a host animal, including a human subject. Nucleic acid sequences comprising “complementary nucleotides” are those which are capable of base-pairing according to the standard Watson-Crick complementarity rules, where guanine pairs with cytosine (G:C) and adenine pairs with either thymine (A:T) in the case of DNA, or adenine pairs with uracil (A:U) in the case of RNA.

While all or part of the gene sequence may be employed in the context of antisense construction, preferably any sequence 17 bases long can be used to specify a unique target sequence. Although shorter oligomers are easier to make and increase in vivo accessibility, numerous other factors are involved in determining the specificity of hybridization. The antisense oligonucleotide is selected such that the binding affinity and sequence specificity to its complementary target is sufficient for use as therapeutic agents. Thus, oligonucleotides of 8, 9, 10, 11, 12, 13, 14, 15, 16, 17, 18, 19, 20, 21, 22 or more base pairs can be used. One can readily determine whether a given antisense nucleic acid is effective at targeting of the corresponding host cell gene simply by testing the constructs in vitro to determine whether the endogenous gene's function is affected or whether the expression of related genes having similar sequences is affected.

### Interfering RNA

Interfering RNA (RNAi) fragments, particularly double-stranded (ds) RNAi, can be used to modulate an arachidonic acid metabolic or signaling pathway. Small interfering RNA (siRNA) are typically 19-25 nucleotide-long RNA molecules that interfere with the expression of genes. Methods relating to the use of RNAi to silence genes in C. elegans, Drosophila, plants, and humans are known in the art [Fire et al., Potent and specific genetic interference by double-stranded RNA in Caenorhabditis elegans. Nature 391: 806-811 (1998); Sharp, RNA interference 2001. Genes and Development. 15: 485-490 (2001); Tuschl, RNA interference and small interfering RNAs. ChemBioChem. 2: 239-245 (2001); WO0129058; and WO9932619].

The nucleotide sequence employed RNAi comprises sequences that are at least about 15 to 50 basepairs. The sequence can be a duplex, optionally with overhangs at the 5′-end and/or the 3′-end, where one strand of the duplex comprises a nucleic acid sequence of at least 15 contiguous bases having a nucleic acid sequence of a nucleic acid molecule within an arachidonic acid metabolic or signaling pathway. The length of each strand can be longer where desired, such as 19, 20, 21, 22, 23, 24, 25, or 30 nucleotides or up to the full length of any of those described herein. The single-stranded overhang can be, for example, 1, 2, 3, 4, 5, or 10 nucleotides long, and can be present at the 3′-end, the 5′ end, or both the 3′-end and the 5′-end. Such fragments can be readily prepared by directly synthesizing the fragment by chemical synthesis, by application of nucleic acid amplification technology, or by introducing selected sequences into recombinant vectors for recombinant production.

In particular, the nucleotide sequences of RNAi can be oligonucleotides complementary to the base sequences of 5-LO, FLAP, LTA4-H, LTBR, and/or LTBR2-encoding DNA and RNA or to the base sequences encoding other proteins in an arachidonic acid metabolism or signaling pathway. The oligonucleotides can be modified or unmodified RNA, DNA, or mixed polymer oligonucleotides, and, when introduced into a target cell, specifically bind to their target nucleic acid and interfere with transcription, RNA processing, transport and/or translation.

### Other Agents

In another aspect of the invention, an additional agent or drug may be administered to the subject. The additional agent can contain one or more active agents that effectively regulate calcium homeostatis, modulate chondrogenesis, modulate osteogenesis, modulate bone remodeling, regulate angiogenesis, regulate pain, regulate inflammation, or have antibiotic activity. The additional active agent can be, but is not limited to, an estrogen, an insulin-like growth factor (IGF), insulin, bone morphogenetic proteins and other growth factors, osteoprotegrin (OPG), a calcitonin, a bisphosphonate, vitamin D₃ or an analogue thereof, a statin, an adrogen, a fluoride salt, a parathyroid hormone or an analogue thereof, agents that enhance angiogenesis such as vascular endothelial growth factor (VEGF), agents that alter regulation of transcription of naturally occurring hormone regulators involved in bone metabolism, a vitamin, a mineral supplement, a nutritional supplement, and combinations thereof. The additional agent also may be an antibiotic such as gentamycin, ciprofloxacin, vancomycin, teicoplanin, tobramycin, and/or others. This additional active agent can be administered to the subject prior to, concurrently with or subsequently to administration of the arachadonic acid pathway modulating compounds of this invention. Anti-inflammatory drugs, including but not limited to nonsteroidal anti-inflammatory drugs and corticosteroids; p38 kinase inhibitors such as SCIO-496, pamapimod and SB-239063: and antiviral drugs, including but not limited to ribivirin, vidarabine, acyclovir and ganciclovir, may also be combined in compositions of the invention. Antibiotic compounds including but not limited to gentamicin, teicoplanin, tobramycin, and vancomycin, may also be combined in the composition of the invention.

### III. Pharmaceutical Formulations and Modes of Administration

The methods described herein use pharmaceutical compositions comprising the molecules described above, together with one or more pharmaceutically acceptable excipients or vehicles, and optionally other therapeutic and/or prophylactic ingredients. Such excipients include liquids such as water, saline, glycerol, polyethyleneglycol, hyaluronic acid, ethanol, cyclodextrins, modified cyclodextrins (i.e., sufobutyl ether cyclodextrins), etc. Suitable excipients for non-liquid formulations are also known to those of skill in the art. Pharmaceutically acceptable salts can be used in the compositions of the present invention and include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients and salts is available in Remington's Pharmaceutical Sciences, 18th Edition (Easton, Pa.: Mack Publishing Company, 1990).

Additionally, auxiliary substances, such as wetting or emulsifying agents, biological buffering substances, surfactants, and the like, may be present in such vehicles. A biological buffer can be virtually any solution which is pharmacologically acceptable and which provides the formulation with the desired pH, such as, a pH in the physiologically acceptable range. Examples of buffer solutions include saline, phosphate buffered saline, Tris buffered saline, Hank's buffered saline, and the like.

Depending on the intended mode of administration, the pharmaceutical compositions may be in the form of solid, semi-solid or liquid dosage forms, such as, for example, tablets, suppositories, pills, capsules, powders, liquids, suspensions, creams, ointments, lotions or the like, preferably in unit dosage form suitable for single administration of a precise dosage. The compositions will include an effective amount of the selected drug in combination with a pharmaceutically acceptable carrier and, in addition, may include other pharmaceutical agents, adjuvants, diluents, buffers, etc.

The invention includes a pharmaceutical composition comprising a compound of the present invention including isomers, racemic or non-racemic mixtures of isomers, or pharmaceutically acceptable salts or solvates thereof together with one or more pharmaceutically acceptable carriers, and optionally other therapeutic and/or prophylactic ingredients.

In general, compounds of this invention will be administered in vivo as pharmaceutical formulations including those suitable for oral (including buccal and sub-lingual), rectal, nasal, topical, pulmonary, vaginal or parenteral (including intramuscular, intraarterial, intrathecal, subcutaneous and intravenous) administration, in a form suitable for administration by inhalation or insufflation, or in a form suitable for administration at the bone formation site. The preferred manner of administration is oral or intravenous using a convenient daily dosage regimen which can be adjusted according to the degree of affliction.

Formulations for delivery in vivo, e.g., locally at the bone formation site, include adsorption onto or encapsulation within polylactide and/or polygalactide polymers, palmitic acid, alginate, plaster, calcium sulfate, calcium phosphate, mixtures of calcium sulfate and calcium phosphate, hydroxyapatite, collagen or other extracellular matrix material, bone wax (such as that from CP Medical, Inc., Ethicon, Inc., Unites States Surgical Corp., or Ceremed), Orthocon Bone Putty (a mixture of calcium stearate, vitamin E acetate, and alkylene oxide copolymer) or other materials or compounds that can be used for this purpose. In vivo delivery can be accomplished by local or direct placement at or in the bone formation site or by deposition of the active compound of the invention with or without a carrier onto the surface of prosthetic or surgically implanted devices.

A pharmaceutically or therapeutically effective amount of the composition is delivered to the subject. The precise effective amount varies from subject to subject and depends upon the species, age, the subject's size and health, the nature and extent of the condition being treated, recommendations of the treating physician, and the therapeutics or combination of therapeutics selected for administration. Thus, the effective amount for a given situation can be determined by routine experimentation. For purposes of the present invention, generally a therapeutic amount will be in the range of about 0.05 mg/kg to about 40 mg/kg body weight, more preferably about 0.5 mg/kg to about 20 mg/kg, in at least one dose. In larger mammals the indicated daily dosage can be from about 1 mg to 4,800 mg, one or more times per day, more preferably in the range of about 10 mg to 1,200 mg. The subject may be administered as many doses as is required to reduce and/or alleviate the signs, symptoms, or causes of the disorder in question, or bring about any other desired alteration of a biological system. One of ordinary skill in the art of treating such diseases will be able, without undue experimentation and in reliance upon personal knowledge and the disclosure of this application, to ascertain a therapeutically effective amount of the compounds of this invention for a given disease. When practicing the methods of the invention starting human doses may need to be estimated from rat dose data. Such estimation methods are well known in the art. See FDA publication “Guidance for Industry: Estimating the Maximum Safe Starting Dose in Initial Clinical Trials for Therapeutics in Adult Healthy Volunteers” published July 2005 (Federal Register Document 5-14456) and available online at the FDA's website listed as Cder guidance pdf document 5541fnl. In general, the rat dose expressed as mg/kg should be divided by 6.2 to obtain an equivalent human dose.

When desired, formulations can be prepared with enteric coatings adapted for sustained or controlled release administration of the active ingredient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Prior human studies using 5-LO inhibitors have shown that 3% to 5% of the patient population experience elevated serum liver enzyme levels. In addition, the activity of 5-LO and FLAP are known to regulate multiple biological processes; and therapies that modify 5-LO and FLAP activity are indicated for other diseases, including respiratory diseases and cardiovascular disease, indicating the diverse and important roles of 5-LO and FLAP in biological processes of various tissues and cell types. Thus, inhibition of LTA4-H or antagonism of the LTB4 receptor using the methods of the present invention to treat a bone fracture, a bone defect, or a condition treated by inducing bone formation may be desirable depending upon the specific circumstances of an individual patient in need of such treatment.

### IV. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### 5-LO Knock Out Mice

Knock out mice lacking 5-lipoxygenase (Alox5−/− or 5-LO−/−) were purchased from Jackson Laboratory, Bar Harbor, Me. An impending femur fracture was stabilized with an intramedullary wire that was inserted retrograde into the femoral canal. A three-point bending device was used to make the fracture. Femur fracture healing was measured or assessed by histomorphometry, radiography, and torsional mechanic testing. The 5-LO−/− mice demonstrated statistically significant, quantitative acceleration and enhancement of fracture healing as compared to wild-type mice of identical genetic background and age (C57BL/6). Closed mid-diaphyseal fractures were made in 10-12 week old female mice. Fracture healing was assessed by x-rays (FIG. 3) and quantitatively assessed by torsional mechanical testing 4 and 12 weeks after fracture (FIG. 4 and TABLE 2). After 4 or 12 weeks of healing, the fractured femurs from 5-LO−/− and wild type (WT) mice were excised and mechanically tested to failure in torsion using an MTS servohydraulic test machine and Interface 20 Nm torque load cell. Fractured femur dimensions were measured before and after testing. Peak torque, rigidity, maximum shear stress, and shear modulus were calculated from callus dimensions and the torque to angular displacement curves. All mechanical parameters were 50-120% higher after 4 weeks of healing in the 5-LO−/− as compared to the WT mice. Histomorphometric analysis of time-staged fracture specimens from normal and 5-LO−/− mice showed that cartilage area peaked early and to a greater extent in the 5-LO−/− mice (FIG. 5 and TABLE 3). Further, significantly more new bone (mineralized tissue) was present in the 5-LO−/− fracture callus at 7 and 10 days after fracture. The data demonstrate that fracture healing is accelerated and enhanced in the 5LO-KO mice.

TABLE 2
Summary of fractured femur torsional mechanical testing data from 5-LO−/− and
wild-type mice of identical genetic background and age at time of fracture (Fx).

| | | | Mean Percentages (Fracture/Contralateral) ± SD | | | |
|---|---|---|---|---|---|---|
| | Sample | Days | Peak | | Max. Shear | Shear |
| Strain | Size | Post-Fx | Torque | Rigidity | Stress | Modulus |
| C57BL/6 | 9 | 28 | 85.3 ± 16.7 | 61.9 ± 31.3 | 18.2 ± 5.4 | 7.8 ± 4.4 |
| C57BL/6 | 6 | 84 | 77.8 ± 20.1 | 110.6 ± 19.1 | 26.2 ± 12.8 | 25.9 ± 13.4 |
| Lox5−/− | 8 | 28 | 128.5 ± 30.3 | 109.9 ± 37.4 | 31.8 ± 9.0 | 17.4 ± 11.1 |
| Lox5−/− | 8 | 84 | 131.4 ± 26.0 | 95.8 ± 37.8 | 49.8 ± 16.9 | 23.2 ± 13.2 |

TABLE 3
Summary of fracture callus histomorphometric analysis from 5-LO−/− and
wild-type mice of identical genetic background and age at time of fracture.

| | Percent Cartilage | | | Percent Mineralized Tissue | | |
|---|---|---|---|---|---|---|
| | (mean ± S.D.) | | | (mean ± S.D.) | | |
| Time Point | Wild-Type | 5-LO−/− | P value | Wild-Type | 5-LO−/− | P value |
| 7 days | 7.84 ± 1.31 | 30.84 ± 3.46 | <0.001 | 12.89 ± 3.76 | 24.56 ± 3.33 | <0.001 |
| 10 days | 20.16 ± 6.13 | 14.46 ± 7.53 | 0.226 | 35.49 ± 9.67 | 47.57 ± 2.86 | 0.028 |
| 14 days | 3.63 ± 1.37 | 2.73 ± 3.71 | 0.625 | 44.66 ± 7.14 | 51.46 ± 5.38 | 0.127 |
| 21 days | 0 ± 0 | 0 ± 0 | 1.000 | 77.26 ± 6.26 | 75.72 ± 2.55 | 0.624 |

The serial x-rays (FIG. 3) show that fracture healing is accelerated in the 5-LO−/− mice as compared to wild type mice (C57BL/6). More specifically, the 10 day old fracture from the 5-LO−/− mouse appears to be at similar stage as the 14 day old fracture from the wild type mouse, the 14 day 5-LO−/− fracture is similar to the 21 day wild type fracture, and the 1 month 5-LO−/− fracture is similar to a 3 month old wild type fracture. The mechanical testing data show quantitatively that the structural and material properties of the 5-LO−/− fracture callus were statistically significantly better than the controls after 4 weeks of healing with a 50% increase in peak torque, a 75% increase in rigidity, a 75% increase in maximum shear stress, and over a 100% increase in shear modulus. Further, the 4 week mechanical testing parameters from the 5-LO−/− mice were similar to those from the 12 week wild type mice, supporting the x-ray data of FIG. 3 and demonstrating that fracture healing was accelerated and enhanced in the 5-LO−/− mice. After 12 week of healing, the rigidity and shear modulus of the wild-type fracture callus had caught-up with the 5-LO−/− fracture callus. Histomorphometric measurements of time-staged fracture callus specimens from the 5-LO−/− and WT mice support the mechanical and radiographic observations (FIG. 5 and TABLE 2). Callus cartilage area peaked by day 7 post-fracture in the 5-LO−/− mice but not until day 10 in the WT mice. There was almost 4-times more cartilage present in the 5-LO−/− callus at day 7 as compared to that from the WT mice. Concurrently, more new bone formation also occurred in the 5-LO−/− mice with almost twice as much new bone (mineralized tissue) present at day 7 and 30% more new bone at day 10 as compared to the WT mice. The data is thus consistent with fracture healing occurring faster and producing more mechanically sound fracture callus with enhanced structural and material properties in the 5-LO−/− mice than in normal mice.

### Example 2

### COX-2 Knockout Mice

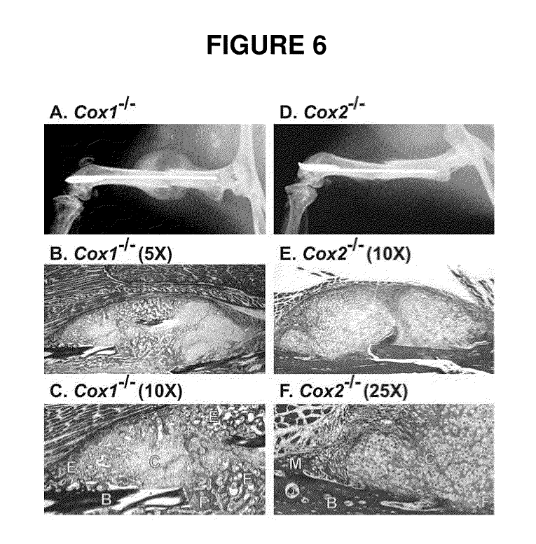

Fracture healing was assayed in mice with a targeted deletion of the COX-2 gene. Closed, mid-diaphyseal femur fractures were made in the right hindlimb of COX-2 knockout, COX-1 knockout, and wild type mice (not shown). Fracture healing was assessed by x-rays and histology (FIG. 6), and by mechanical testing (not shown). The data show that fracture healing was dramatically impaired in the COX-2 knockout mice, but not the COX-1 knockout or wild type mice. X-rays after 14 days of healing show a large mineralized fracture callus in the COX-1 knockout mouse (FIG. 6) with little or no evident mineralized callus in the COX-2 knockout mouse. Histological examination confirmed the x-ray findings in that the COX-2 knockout callus had a significant amount of cartilage but no new bone was evident. Torsional mechanical testing data shows that fracture callus structural and material properties are significantly worse than COX-1 knockout or wild type mice. When combined with the experimental results of example 1, example 3, and example 4 this demonstrates that COX-2 activity is a positive-regulator of fracture healing, that 5-LO activity is a negative-regulator of fracture healing, and that arachidonic acid metabolism or signaling can be manipulated according to the methods of the invention to affect bone formation.

### Example 3

### Treatment of Rats with a 5-Lipoxygenase Inhibitor

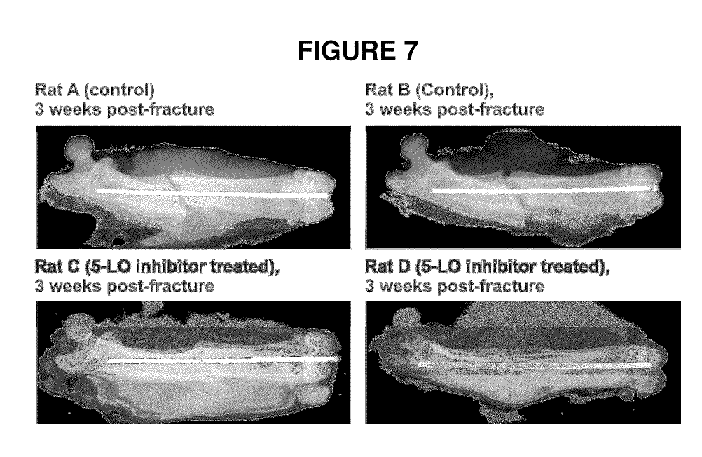

Sprague-Dawley rats (3 months old) underwent a standard closed femur fracture procedure as described in the art [Simon et al., Cyclo-oxygenase 2 function is essential for bone fracture healing. Journal of Bone and Mineral Research, 17: 963-976 (2002); Bonnarens and Einhorn, Production of a standard closed fracture in laboratory animal bone. Journal of Orthopaedic Research, 2: 97-101 (1984)]. The impending fracture was stabilized with an intramedullary stainless steel pin. Beginning 4 hours after fracture the rats were treated with 30 mg/kg of NDGA (nordihydrogaiaretic acid) in 1% methylcellulose (5-lipoxygenase inhibitor treatment group) or with carrier only (1% methylcellulose). The day after surgery and continuing until day 14 post-fracture, experimental rats were treated with 2 doses of NDGA (30 mg/kg), the first dose between 8-LOAM and then again with another NDGA dose 8-10 hours later. Control rats were treated similarly but with carrier only (1% methylcellulose). Three weeks after fracture, the rats were sacrificed, the fractured femurs were harvested, and high resolution radiographs were made of the fractured femurs using a Packard Faxitron and Kodak MinR2000 mammography film. Two representative radiographs are shown in FIG. 7 for each treatment group: control and 5-lipoxygenase (5-LO) inhibitor treated.

The radiographs show that after 3 weeks the fractured femurs of the 5-LO inhibitor treated rats were bridged with new bone. In contrast, a well-formed, mineralized fracture callus has formed in the control rats but the fracture site had not yet bridged with new bone. In rat C, the fracture is bridged with new bone on the medial (top) and lateral (bottom) sides of the fracture callus. In rat D, the fracture is bridged with new bone on the lateral side (bottom) and shows indications of new bone bridging on the medial side. No new bone bridging is evident in the control rats (rats A and B). The data thus demonstrates that 5-LO inhibitor therapy can accelerate the fracture healing process in young, normal rats.

### Example 4

### Treatment of Rats with 5-Lipoxygenase Inhibitors

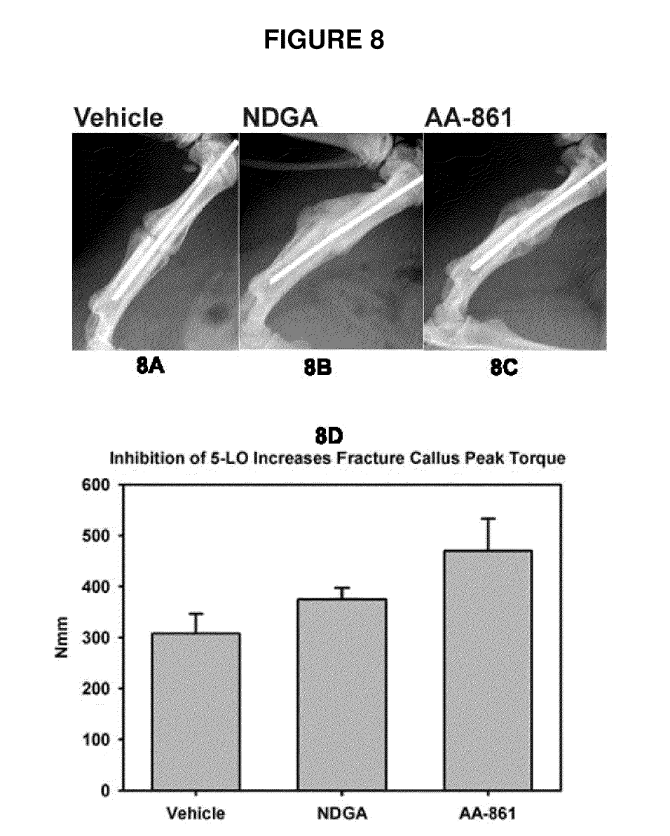

Sprague-Dawley rats (3 months old) underwent a standard closed femur fracture procedure as described in the art (Simon et al., Cyclo-oxygenase 2 function is essential for bone fracture healing. Journal of Bone and Mineral Research, 17: 963-976 (2002); Bonnarens and Einhorn, Production of a standard closed fracture in laboratory animal bone. Journal of Orthopaedic Research, 2: 97-101 (1984)]. The impending fracture was stabilized with an intramedullary stainless steel pin. Beginning 4 hours after fracture the rats were treated with vehicle (1% methylcellulose) or inhibitors of 5-LO suspended in 1% methylcellulose. Inhibitor A (NDGA) was administered at 30 mg/kg and Inhibitor B (AA-861) was administered at 5 mg/kg. The day after surgery and continuing until day 21 post-fracture, experimental rats were treated with 2 doses of inhibitor (either A or B), the first dose between 8-10 AM and then again with another dose 8-10 hours later. Control rats were treated similarly but with carrier only (1% methylcellulose). Three weeks after fracture the rats were anesthetized and high resolution radiographs were made of the fractured femurs using a Packard Faxitron and Kodak MinR2000 mammography film (FIGS. 8A, 8B, and 8C). Five weeks after fracture the rats were sacrificed, femurs resected, and assayed for structural mechanical properties by torsional mechanical testing (FIG. 8D).

The radiographs showed that after 3 weeks of healing, the fractures appeared bridged in the 5-LO inhibitor treated rats but not in the vehicle treated rat.

Torsional mechanical testing was used to measure the peak torque sustained by each femur after 5 weeks of healing. The data show that the femurs from the Inhibitor A (NDGA) treated rats and from the Inhibitor B treated rats had 22% and 53% greater peak torque than vehicle treated rats (FIG. 8D). In addition, all of the femurs from the Inhibitor A or B treated rats failed as boney unions while 13% (2 of 15) of the femurs from the vehicle treated rats failed as non-unions with no apparent bone bridging.

These experimental observations demonstrate that 5-LO inhibition therapy can accelerate (faster bone bridging) and enhance (better mechanical properties) fracture healing.

### Example 5

### Ex Vivo Treatment Methods Using Small Molecule Compounds, RNAi, and Antisense Compounds

Methods to promote ex vivo osteogenesis are used, e.g., to aid in healing of recalcitrant bone fractures or segmental defects caused by traumatic injuries or pathological resection of bone segments, or for joint arthrodesis. In these instances, precursor bone cells are isolated from a subject or from a suitable donor and are cultured ex vivo using standard methods [discussed by Frolich et al. Tissue engineered bone grafts: biological requirements, tissue culture and clinical relevance. Current Stem Cell Research & Therapy 3:254-264 (2008)]. The cells are grown in or seeded into an appropriate scaffold that either represents the segment of missing bone or can be molded to fit the missing segment or juxtapose the ends of the bone. The cells are induced to form bone ex vivo using appropriate cell culture conditions or with inductive factors, such as bone morphogenetic protein-2 (BMP-2). Once the cells have met appropriate criteria known to one skilled in the art, such as elaboration of a bone matrix, the construct can be implanted into the patient to affect osteogenesis and promote healing. This sequence of events is typically referred to as a tissue engineering approach to enhancing osteogenesis.

Inhibition of 5-lipoxygenase (5-LO), FLAP, LTA4-hydrolase, LTBR, and/or LTBR2 can be used to promote ex vivo osteogenesis. As demonstrated in EXAMPLES 1, 3, 4, 7, 8, 9, 10, and 11, 5-LO activity or signaling and LTA4-hydrolase activity or signaling negatively regulate osteogenesis. Thus osteogenesis can be promoted ex vivo with small molecule inhibitors of 5-LO, FLAP, LTA4-hydrolase, LTBR, and/or LTBR2 alone, in combination with each other, or in combination with well known osteo-inductive or osteo-promotive agents, such as statins, PTH or its derivatives, FGF-2, BMP-2, and/or PDGF by one of ordinary skill in the art.

Similarly, RNAi mediated inhibition of 5-LO, FLAP, LTA4-hydrolase, LTBR, and/or LTBR2 activity can be used to promote ex vivo osteogenesis. This is accomplished by transfecting cells with pools of siRNA sequences using commercially available transfection reagents, such as TransIT-TKO or jetSI. Approximately 1 million cells can be transfected with one or more siRNAs specific for 5-LO, FLAP, LTA4-hydrolase, LTBR, and/or LTBR2 using 50-200 pmoles of each siRNA. Alternatively, a pool of siRNAs that target 5-LO, FLAP, LTA4-hydrolase, LTBR, and/or LTBR2 in combination can be used.

One skilled in the art would recognize that in addition to direct transfection of the siRNAs into cells, expression vectors can be developed that express these or similar sequences and the expression vectors delivered to the cells by transfection, viral mediated delivery, or methods for delivering DNA molecules into cells. The expression vectors express the siRNAs leading to sustained inhibition of 5-LO, FLAP, LTA4-hydrolase, LTBR, or LTBR2 alone or in combination and thereby promote osteogenesis.

Mehrabian et al. [Identification of ALOX5 as a gene regulating adiposity and pancreatic function. Diabetologia 51:978-988 (2008)] teaches use of RNAi technology to inhibit 5-LO activity in human pancreatic islets cells. Ihara et al. [Blockade of leukotriene B4 signaling pathway induces apoptosis and suppresses cell proliferation in colon cancer. Journal of Pharmacological Sciences 103:24-32 (2007)] teaches use of RNAi technology to inhibit LTBR activity in human colon cancer cell lines. Thus one of ordinary skill in the art can adapt these methods of inhibiting 5-LO, FLAP, LTA4-hydrolase, LTBR, and/or LTBR2 inhibition to use for the ex vivo enhancement of osteogenesis based upon methods described herein.

The treated cells can be cultured and osteogenesis assessed as extracellular matrix production of cartilage or bone matrix using methods familiar to one skilled in the art such as alcian blue or alizarin red binding as appropriate, by measurement of specific matrix protein, or by measurement of osteogenesis following implantation into a subject in need thereof.

Pools of siRNA pairs for 5-LO can be chosen, e.g., from POOL-A (5′-AAC TGG GCG AGA TCC AGC TGG-3′ (SEQ ID NO: 9), 5′-AAG CTC CCG GTG ACC ACG GAG-3′ (SEQ ID NO: 10), 5′-AAG GAA GCC ATG GCC CGA TTC-3′) (SEQ ID NO: 11), POOL-B (5′-AAT CGA GAA GCG CAA GTA CTG-3′ (SEQ ID NO: 12), 5′-AAG GAG TGG ACT TTG TTC TGA-3′ (SEQ ID NO: 13), 5′-AAC TTC GGC CAG TAC GAC TGG-3′) (SEQ ID NO: 14), or POOL-C (5′-AAG TTG GCC CGA GAT GAC CAA-3′ (SEQ ID NO: 15), 5′-AAC ACA TCT GGT GTC TGA GGT-3′ (SEQ ID NO: 16), 5′-AAC CAT GCG AGC CCC GCC ACC-3′) (SEQ ID NO: 17). Pools of siRNA pairs for FLAP can be chosen, e.g., from POOL-D (5′-AAG CAA ACA TGG ATC AAG AAA-3′ (SEQ ID NO: 18), 5′-AAG TTC CTG CTG CGT TTG CTG-3′ (SEQ ID NO: 19), 5′-AAT TCA GCT CTT GAG AGC ATT-3′) (SEQ ID NO: 20), POOL-E (5′-AAT GGA TTC TTT GCC CAT AAA-3′ (SEQ ID NO: 21), 5′-AAG TAC TTT GTC GGT TAC CTA-3′ (SEQ ID NO: 22), 5′-AAT CTA TTG GCC ATC TGG GCT-3′) (SEQ ID NO: 23), or POOL-F (5′-AAC CAG AAC TGT GTA GAT GCG-3′ (SEQ ID NO: 24), 5′-AAG TGA CTT TGA AAA CTA CAT-3′ (SEQ ID NO: 25), 5′-AAT GAT GTC ATG TCA GCT CCG-3′) (SEQ ID NO: 26). Pools of siRNA pairs for LTA4-H can be chosen, e.g., from POOL-G (5′-AGA AAG AGC AGG TGG AAA A-3′(SEQ ID NO: 27), 5′-CAA ATA TGC TCT TGG AGA A-3′(SEQ ID NO: 28), 5′-GGA CAC TCC TTC TGT GAA A-3′(SEQ ID NO: 29)), POOL-H (5′-CTA AAG AAC TGG TGG CAC T-3′(SEQ ID NO: 30), 5′-TGA CAA ATC CCA TGA TCA A-3′(SEQ ID NO: 31), 5′-GGA GAA AGA CAA AGT TAC A-3′(SEQ ID NO: 32)), or POOL-I (5′-AGA TAT AGA CCC TGA TGT A-3′(SEQ ID NO: 33), 5′-GCT TGG AGG ACC AGA GAT T-3′ (SEQ ID NO: 34), 5′-CCA CAG ACC TGA AGG ATC T-3′ (SEQ ID NO: 35)). Pools of siRNA pairs for LTBR can be chosen, e.g., from POOL-J (5′-GGA CAT AGG GCG TCG GCT A-3′(SEQ ID NO: 36), 5′-GGT TAG GGC TCG TGG GGA A-3′(SEQ ID NO: 37), 5′-GGA CAG TAG TGC CCT GGA A-3′(SEQ ID NO: 38)), POOL-K (5′-GCT TTG TGG TGT GGA GTA T-3′ (SEQ ID NO: 39), 5′-GGT GTG GAG TAT CCT GAA A-3′(SEQ ID NO: 40), 5′-GCC CAA GGC ACC TGG AGT T-3′(SEQ ID NO: 41)), or POOL-L (5′-CTC ACT AGG TGT AGA GTT C-3′ (SEQ ID NO: 42), 5′-GCA TCT GGG TGT TGTCCT T-3′ (SEQ ID NO: 43), 5′-GCG TGA ACC CCG TGC TGT A-3′(SEQ ID NO: 44)). Pools of siRNA pairs for LTBR2 can be chosen, e.g., from POOL-M (5′-GCT GCA AGG CGG TGT ACT A-3′(SEQ ID NO: 45), 5′-CTG CTG AGC TGG AAG ACT T-3′(SEQ ID NO: 46), 5′-GCG TCA ACC CGG TGC TCT A-3′(SEQ ID NO: 47)), POOL-N (5′-GAA GGA TGT CGG TCT GCT A-3′ (SEQ ID NO: 48), 5′-GGG AAG GGA CCA TGG AGC T-3′(SEQ ID NO: 49), 5′-GCG TAT GCC AGC TGT GCC A-3′(SEQ ID NO: 50)), or POOL-0 (5′-GGG GTG GGA TGG AGA AGG A-3′(SEQ ID NO: 51), 5′-GCA CCT TCT CAT CGG GCA T-3′ (SEQ ID NO: 52), 5′-CGT CTT CAC CGC TGG AGA T-3′ (SEQ ID NO: 53)). Additional siRNA sequences targeted against the above noted mRNAs could be identified by one of ordinary skill in the art using various prediction tools, e.g., the Dharmacon siDesignCenter tool as available for use on the Dharmacon web-site on Feb. 21, 2008. For brevity, only the sense strand of each siRNA pair is shown. It is well known in the art that siRNA pairs are double stranded small RNAs that have a 5′-AA overhang on the sense strand and a 5′-UU overhang on the antisense strand. It also is well known in the art that backbone chemistry modifications can be advantageous for stabilizing or improving the uptake of the siRNA molecules. Pirollo K F et al., (2003), Rait A, Sleer L S, Chang E H, “Antisense therapeutics: from theory to clinical practice,” Pharmacol Ther. 99(1):55-77. Manufacture of oligonucleotides with advantageous backbone chemistry modifications is within the level of ordinary skill, and use of such modified-backbone compounds (as well as non-modified-backbone compounds) is within the scope of the present invention.

One skilled in the art also will recognize that additional strategies to inhibit expression of 5-LO, FLAP, LTA4-hydrolase, LTBR, or LTBR2 can be used to promote the same osteogenic effects in precursor skeletal cells. Such technologies include use of anti-sense.

Exemplary 5-Lipoxygenase anti-sense sequences include, e.g., 5′-GCA GGT GCT TCT CGC TGC AGC C-3′ (SEQ ID NO: 54), 5′-GCC AGT ACT TGC GCT TCT CG-3′ (SEQ ID NO: 55), 5′-CCA TCG ATA TTG TTT TTG CC-3′ (SEQ ID NO: 56), 5′-GGA GCT TCT CGG GCA GCT CTG TGC-3′ (SEQ ID NO: 57), 5′-CCA GGT TCT TAT ACA GCA AGC-3′ (SEQ ID NO: 58), 5′-CCA GCA GCT TGA AAA TGG GGT GC-3′ (SEQ ID NO: 59), 5′-GCC CCG GGC CTT GAT GGC C-3′ (SEQ ID NO: 60), 5′-CCA CGC CCT TGG CAG TCG G-3′ (SEQ ID NO: 61), and 5′-GCG GAA TCG GGC CAT GGC TTC C-3′ (SEQ ID NO: 62).

Exemplary FLAP anti-sense sequences include, e.g., 5′-GTT CCG GTC CTC TGG AAG CTC C-3′ (SEQ ID NO: 63), 5′-CGC AGA CCA GAG CAC AGC G-3′ (SEQ ID NO: 64), 5′-GCA AAC GCA GCA GGA AC-3′ (SEQ ID NO: 65), 5′-CGT TTC CCA AAT ATG TAG CC-3′ (SEQ ID NO: 66), 5′-GTT TTC AAA GTC ACT TCC G-3′ (SEQ ID NO: 67), 5′-GGT TAA CTC AAG CTG TGA AGC-3′ (SEQ ID NO: 68), 5′-GGA GCT GAC ATG ACA TC-3′ (SEQ ID NO: 69), and 5′-GGC CAC GGT CAT GTT CAA GG-3′ (SEQ ID NO: 70).

Exemplary LTA4-H anti-sense sequences include, e.g., 5′-CTG CTT GGG TCT TCT GGG TC-3′(SEQ ID NO: 71), 5′-CTG CTT GGG TCT TCT GGG TCA-3′(SEQ ID NO: 72), 5′-CCT GCT TGG GTC TTC TGG GT-3′(SEQ ID NO: 73), 5′-TTT CCA CCT GCT CTT TCT CA-3′(SEQ ID NO: 74), 5′-GCT TGG GTC TTC TGG GTC A-3′(SEQ ID NO: 75).

Exemplary LTBR anti-sense sequences include, e.g., 5′-CTC TCC CTC TTC TTC CAC TCC-3′(SEQ ID NO: 76), 5′-CCT CTC CCT CTT CTT CCA CTC-3′(SEQ ID NO: 77), 5′-TCT CCC TCT TCT TCC ACT CC-3′(SEQ ID NO: 78), 5′-TCT CCC TCT TCT TCC ACT CCA-3′(SEQ ID NO: 79), 5′-TCC ACC TCT CCC TCT TCT TCC-3′(SEQ ID NO: 80).

Exemplary LTBR2 anti-sense sequences include, e.g., 5′-TCC TAC CTC CTC CCA CCT CT-3′(SEQ ID NO: 81), 5′-TCC TAC CTC CTC CCA CCT CTT-3′(SEQ ID NO: 82), 5′-CCT ACC TCC TCC CAC CTC TT-3′(SEQ ID NO: 83), 5′-CTA CCT CCT CCC ACC TCT T-3′(SEQ ID NO: 84), 5′-TCC TCC CAC CTC TTG CCT CA-3′(SEQ ID NO: 85).

### Example 6

### Ex Vivo Treatment of Platelet-Rich Plasma (PrP) with Compounds that Inhibit 5-Lipoxygenase Activity

Platelet-rich plasma is used clinically to augment fracture healing and other osteogenic processes [Wrotniak et al. Current opinion about using the platelet-rich gel in orthopaedics and trauma surgery. Ortopedia, Traumatologia, Rehabilitacja 9:227-238 (2007)]. Platelet-rich plasma is enriched with platelets and white blood cells from whole blood by centrifugal separation. White blood cells, such as, macrophages, monocytes, and neutrophils, are a major source of 5-lipoxygenase activity [Woods et al. 5-lipoxygenase and 5-lipoxygenase-activating protein are localized in the nuclear envelope of activated human leukocytes. J. Exp. Med. 178:1935-1946 (1993)]. Since 5-lipoxygenase and LTA4-hydrolase activity are negative regulators of fracture healing (see EXAMPLES 1, 3, and 4 above and EXAMPLES 9 and 10 below), it follows that significantly reducing the 5-lipoxygenase or LTA4-hydrolase activity in platelet-rich plasma by treatment with a leukotriene pathway modifier, including 5-LO, FLAP and LTA4 hydrolase inhibitors or LTB4 receptor antagonists, would have a beneficial effect on osteogenic applications that use platelet-rich plasma. This was tested by showing that ex vivo treatment of rat platelet-rich plasma significantly reduces leukotriene B4 levels, which is an indicator of 5-lipoxygenase activity.

Male Sprague-Dawley rats (450-500 g body weight) were used. Platelet-rich plasma was prepared as described previously using differential centrifugation [Gandhi et al. The effects of local platelet rich plasma delivery on diabetic fracture healing. Bone 38:54-546 (2006)]. Aliquots of platelet-rich plasma (0.2 ml) were treated with 5-LO inhibitors [zileuton (1 ug/ml) or AA-861 (1 ug/ml)], a FLAP inhibitor [MK-886 (1 ug/ml)], an LTA4-hydrolase inhibitor [SC-22716 (1 ug/ml)], or an equivalent volume of vehicle [dimethyl sulfoxide] that was used as the control. Five minutes after drug treatment, the treated platelet-rich plasma was activated by addition of thrombin (100 units/ml) and calcium chloride (10 mg/ml) followed by incubation for 15 minutes at 37° C. The reaction was stopped and the platelet-rich plasma was separated by high-speed centrifugation (14,000 RPM). The supernatant was collected and assayed for leukotriene B4 levels using a commercial enzyme-linked immune-assay from Cayman Chemicals, Ann Arbor, Mich.

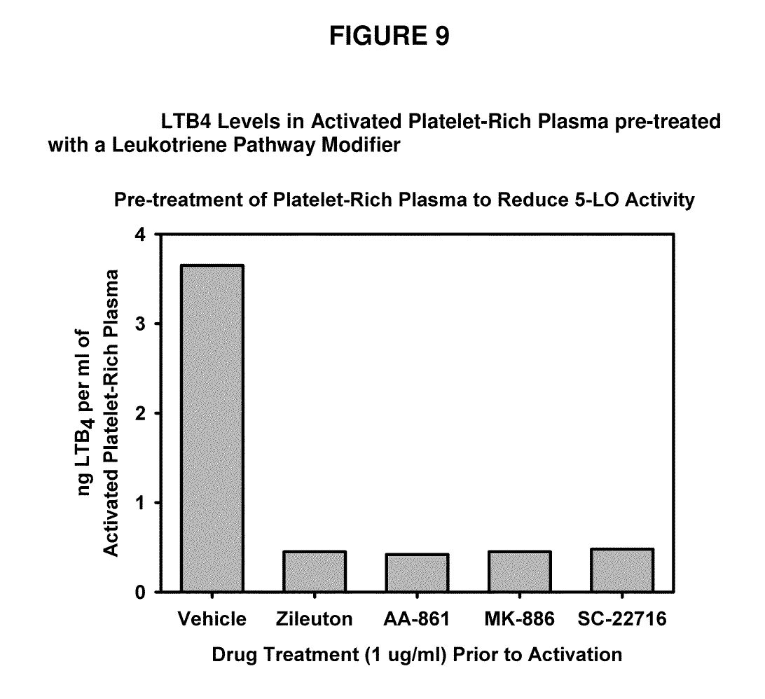

As seen in FIG. 9, activated platelet-rich plasma had very high LTB4 levels (over 3,000 pg/ml). Conversely, ex vivo treatment of platelet-rich plasma with inhibitors of 5-LO, FLAP, or LTA4-hydrolase led to greater than 6-fold reductions in activated platelet-rich plasma LTB4 levels. The reduced LTB4 levels observed in the ex vivo treated platelet-rich plasma is indicative of reduced 5-LO or LTA4-hydrolase activity and, as shown herein, improved osteogenic activity. Thus, platelet-rich plasma can be used to promote or enhance osteogenesis by incorporating an ex vivo treatment step in which 5-LO activity, LTA4-hydrolase activity, and/or LTB4 receptor activity in a platelet-rich plasma preparation is reduced prior to, concomitant with the administration of the preparation to a subject in need of osteogenic treatment. Alternatively, or additionally, 5-LO activity, LTA4-hydrolase activity, and/or LTB4 receptor activity in the platelet-rich plasma preparation may be reduced subsequent to administration to the patient.

### Example 7

### Ex Vivo Treatment of Bone Marrow Cells with Compounds that Inhibit 5-Lipoxygenase Activity

Critical-size (8 mm) segmental defects were made in the right femur of 450-500 g male Sprague-Dawley rats. The defects were repaired as described by Rai et al. [Combination of platelet-rich plasma with polycaprolactone-tricalcium phosphate scaffolds for segmental defect repair. Journal of Biomedical Materials Research 81A:888-899 (2007)] except the polycaprolactone-tricalcium phosphate scaffolds were filled with autologous bone marrow that was treated ex vivo as described below rather than with platelet-rich plasma.

Following induction of anesthesia, bone marrow was harvested from the left femur of each rat. A medial peripatellar incision was made to the left femur and the patella was dislocated laterally to provide access to the femoral condyles. Using an 18 gauge needle, a hole was drilled into the femoral canal and the bone marrow collected into the bore of the 18 gauge needle. The marrow was forced from the syringe needle into a small test tube using 100 μl of saline containing 20 units of heparin. The harvested marrow was then treated with the dimethyl sulfoxide (DMSO) or AA-861 (1 μl of 100 mg/ml in DMSO) which is a 5-LO inhibitor. After 5 minutes, 20 μl of a thrombin and CaCl₂ solution (1,000 units thrombin per ml in 10% CaCl₂) was added to each marrow sample. Each marrow sample was gently mixed and applied to a polycaprolactone-tricalcium phosphate scaffold that was set upright in a 0.2 ml tube. Each marrow-laden scaffold was incubated at room temperature for 10 minutes to allow clotting to occur and was then inserted into the femoral defects.

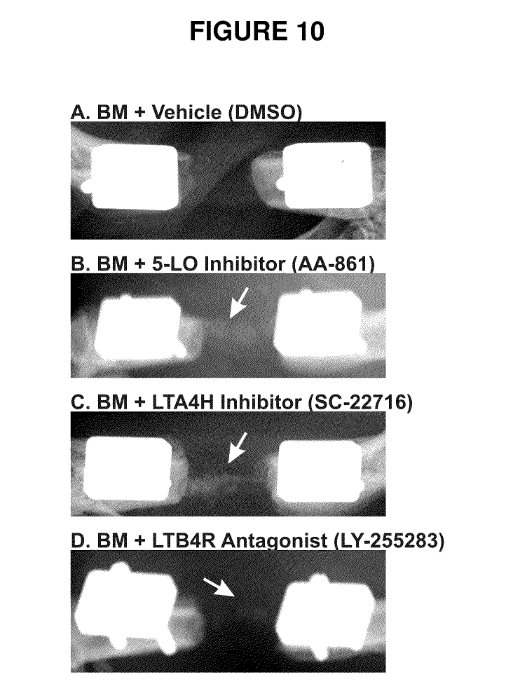

Healing was assessed by radiographic examination 2 weeks after implantation (FIGS. 10A and B). The data show ex vivo treatment of bone marrow aspirate with a 5-LO inhibitor (AA-861), and thus the stem cells contained within that marrow, accelerated and enhanced healing of the femur segmental defect as compared to the vehicle-bone marrow scaffold control group. After just 2 weeks, the radiographs show more extensive mineralized tissue in the femur segmental defects of the rats treated with scaffold laden with marrow aspirates that had been treated ex vivo with a 5-LO inhibitor (AA-861) as compared to the rats that had been treated with the scaffold laden with marrow treated ex vivo with the vehicle control. The data indicate that ex vivo treatment of cells with a 5-LO inhibitor can enhance osteogenesis in vivo.

### Example 8

### Ex Vivo Treatment of Bone Marrow Cells with 5-Lipoxygenase Pathway Modifiers

Critical-size femoral defects were made in male Sprague-Dawley rats as described above in EXAMPLE 7. The defects were treated with bone marrow laden scaffolds as described above. In this experiment however, the bone marrow was treated ex vivo with (a) dimethyl sulfoxide (DMSO) as the vehicle control (FIG. 10A), (b) with SC-22716 (5 μl of a 10 mg/ml solution in DMSO) which is a leukotriene A4 hydrolase inhibitor [Penning et al. Structure-activity relationship studies on 1-[2-(4-Phenylphenoxy)ethyl]pyrrolidine (SC-22716), a potent inhibitor of leukotriene A(4) (LTA(4)) hydrolase. Journal of Medicinal Chemistry 43:721-735 (2000)] (FIG. 10C), or with (c) LY-255283 (5 μl of a 1 mg/ml solution in DMSO) which is a leukotriene B4 receptor antagonist [Herron et al. Leukotriene B4 receptor antagonists: the LY255283 series of hydroxyacetophenones. Journal of Medicinal Chemistry 35:1818-1828 (1992)] (FIG. 10D).

Healing was measured by radiography 2 weeks after surgery (FIG. 10). The data show that ex vivo treatment of bone marrow aspirate, and thus the stem cells contained within that marrow, accelerated and enhanced healing of the femur segmental defect as compared to the vehicle-bone marrow scaffold control group. The radiographs show more extensive mineralized tissue in the femur segmental defect of the rat treated with marrow aspirates that had been treated ex vivo with an LTA4-hydrolase inhibitor (SC-22716) (FIG. 10C), with some evident mineralized tissue in the rat treated with marrow aspirates that had been treated ex vivo with an LTB4 receptor antagonist (LY-255283) (FIG. 10D), but no evident mineralized tissue in the vehicle-bone marrow scaffold control sample (FIG. 10A). The data indicate that ex vivo treatment of cells with a 5-LO pathway modifier (LTA4-hydrolase inhibitor or LTB4 receptor antagonist) can enhance osteogenesis in vivo.

### Example 9

### LTA4 Hydrolase Knockout Mice

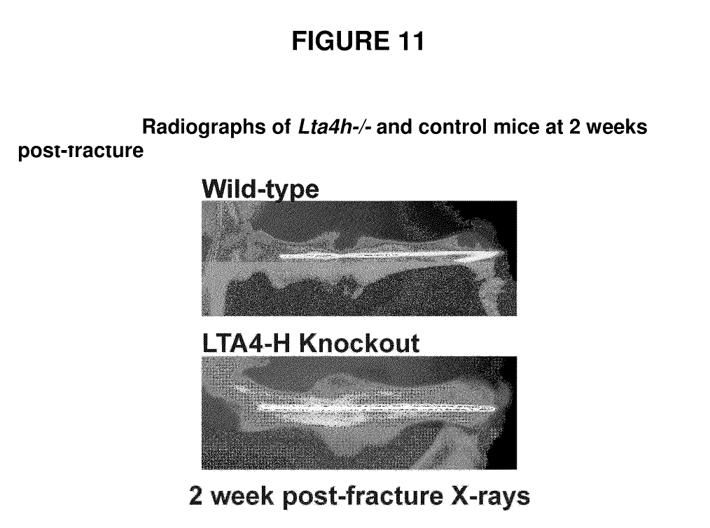

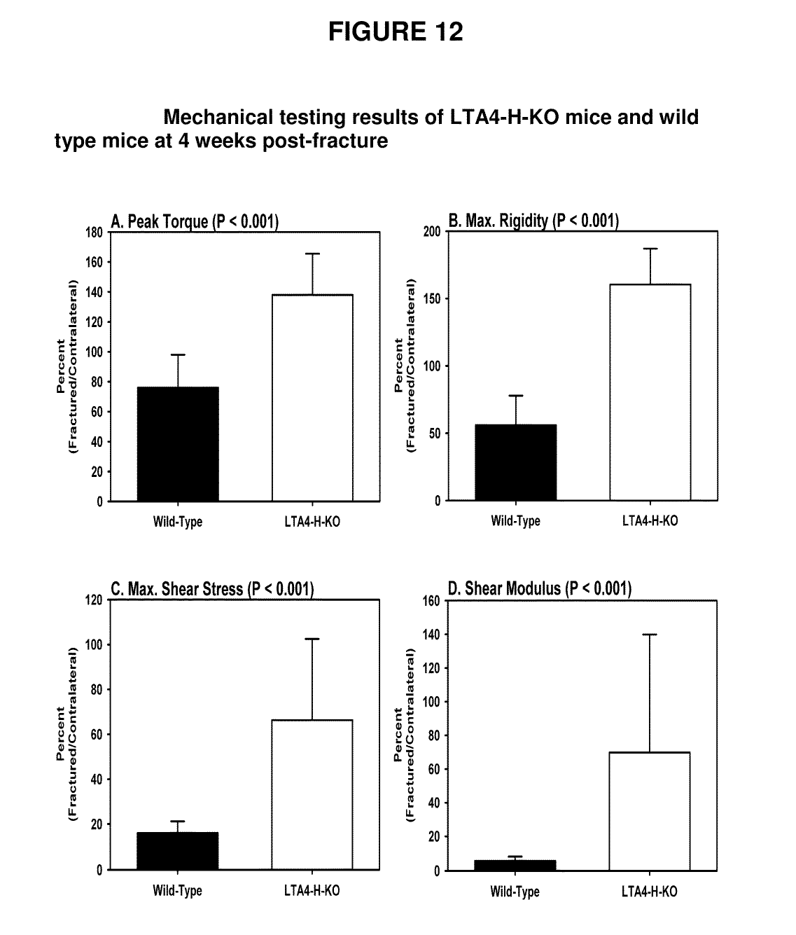

Mice homozygous for a targeted mutation in the LTA4-hydrolase gene (Lta4h−/−) were purchased from Jackson Laboratory, Bar Harbor, Me. and used to establish a breeding colony of LTA4-hydrolase knockout mice. Closed fractures of the right femur diaphysis were made in female Lta4h−/− mice using a three-point bending device and previously established methods [Manigrasso and O'Connor, Characterization of a closed femur fracture model in mice. Journal of Orthopaedic Trauma 18:687-695 (2004)]. The fractures were stabilized with an intramedullary wire that was inserted retrograde into the femoral canal. Healing of the fractures was assessed by radiography and measured by torsional mechanical testing. Based upon radiographic observations, fracture healing proceeds significantly faster in the Lta4h−/− mice as compared to the controls (FIG. 11). While a large fracture callus is formed in control, wild-type mice by 14 days after fracture, the fracture callus in the Lta4h−/− mice is already bridged with new bone by 14 days after fracture which is indicative of accelerated healing. Torsional mechanical testing was performed on femurs from control, wild-type mice (black bars) or Lta4h−/− mice (white bars) that were harvested 4 weeks after fracture. Values from the fractured femur of each mouse were normalized to the contralateral, intact femur values for that mouse as a percentage. The normalized values were compared between the wild-type and Lta4h−/− mice using t-tests. The data shown in FIG. 12 demonstrate that the healing femurs from the Lta4h−/− mice had significantly greater structural (peak torque and maximum rigidity) and material (maximum shear stress and shear modulus) properties as compared to the wild-type mice femurs. Thus, loss of LTA4-hydrolase activity leads to accelerated healing and enhanced biomechanical properties. These data show that LTA4-hydrolase negatively regulates osteogenesis.

### Example 10

### Treatment of Rats with a Leukotriene A4 Hydrolase Inhibitor

Male Sprague-Dawley rats underwent a standard closed fracture of the right femur (see above). Impending fractures were stabilized with metal rods that were inserted retrograde into the femoral canal. Mid-diaphyseal fractures were made using a 3-point bending device. Beginning 4 hours after fracture, rats were treated with twice daily, oral doses of captopril (an LTA4-hydrolase inhibitor, at 30 mg/kg) or the vehicle (1% methylcellulose) which acted as the control group.

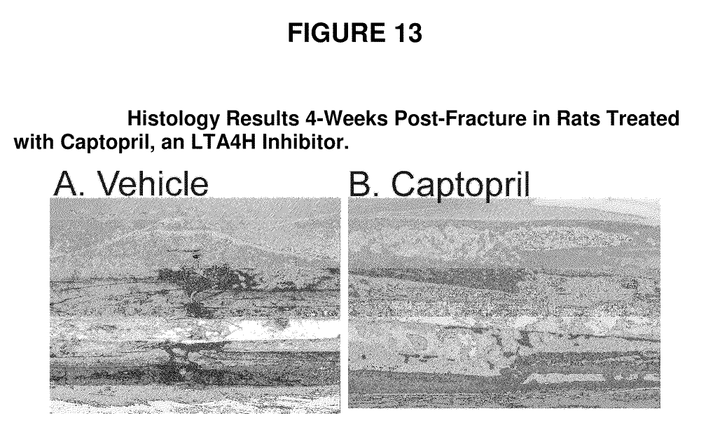

Histological examination of a vehicle-treated rat callus at 4 weeks after fracture showed a normal callus that appeared to be partially bridged with new bone (FIG. 13A). One side of the control callus appeared to be bridged with bone while the opposite side still had evident cartilage and had not fully bridged with new bone. In contrast, histological examination of fracture callus from a captropril-treated rat after 4 weeks of healing showed a fully bridged callus that had already significantly remodeled based upon the smaller callus size and increased thickness of the callus peripheral bone (FIG. 13B).

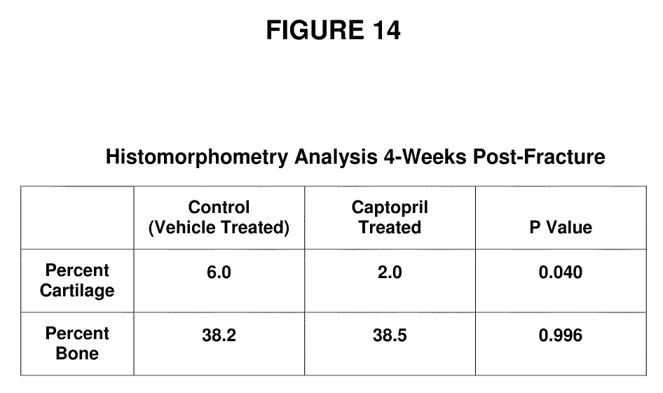

Using histomorphometric analysis, the callus, mineralized tissue and cartilage areas of the histology samples were measured. The analysis shows that, at 4 weeks post-fracture, the captopril treatment group had 66% less cartilage than the control group (FIG. 14).

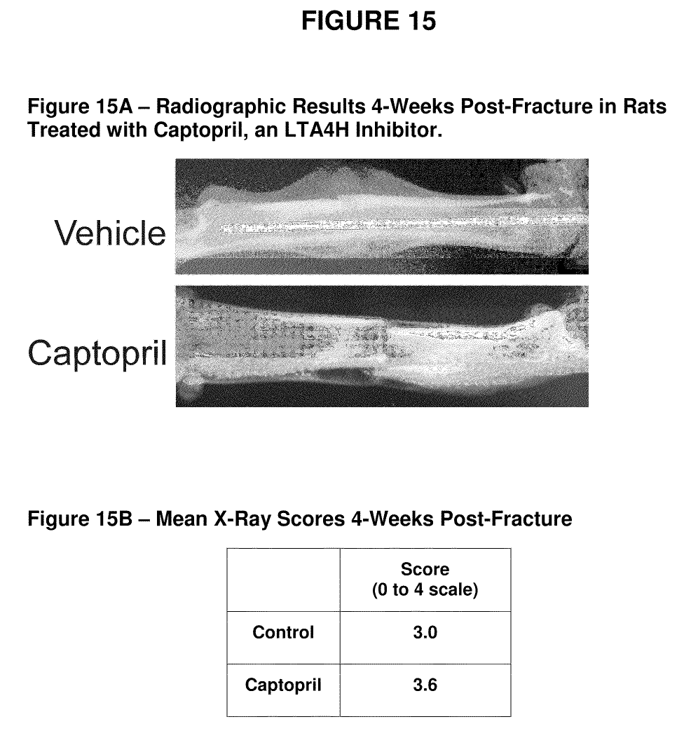

Radiographic examination of the healing femurs also indicated that captopril treatment accelerated healing (FIG. 15A). While a large callus was evident in the vehicle-treated rat that is typical of healing in this species, the fracture callus in the captopril-treated rat was fully bridged and had already significantly remodeled. In addition, each radiograph was scored from 0 to 4 based on apparent bone bridging across the fracture callus at the left and right periphery (1 point each) and apparent bone bridging between the cortices of the femur on the left and right sides (1 point each). Mean radiographic scores for the captopril treated rats was 3.6 as compared to 3.0 for the control rats at 4 weeks after fracture (FIG. 15B).

These data show that pharmacological inhibition of LTA4-hydrolase activity can accelerate osteogenic processes and indicate that LTA4-hydrolase is a negative regulator of ostegenesis.

### Example 11

### Treatment of Rat Femur Segmental Defects with Leukotriene Pathway Modifiers

Male Sprague-Dawley rats underwent surgery during which an 8 mm diaphyseal, segmental defect in the right femur was made as described by Rai et al. [Combination of platelet-rich plasma with polycaprolactone-tricalcium phosphate scaffolds for segmental bone defect repair. Journal of Biomedical Materials Research 81A:888-899 (2007)]. The defect was repaired with a polycaprolactone-tricalcium phosphate scaffold (8 mm long×3 mm diameter; Osteopore International, Singapore) or the scaffold impregnated with calcium sulfate containing a leukotriene pathway modifier. The leukotriene pathway modifiers used were AA-861 (5% w/w), a 5-LO inhibitor; MK-886 (5% w/w), a FLAP inhibitor; SC-22716 (1% w/w), an LTA4-hydrolase inhibitor [Penning et al. Structure-activity relationship studies on 1-[2-(4-Phenylphenoxy)ethyl]pyrrolidine (SC-22716), a potent inhibitor of leukotriene A(4) (LTA(4)) hydrolase. Journal of Medicinal Chemistry 43:721-735 (2000)]; and LY-255283 (1% w/w), a potent and specific leukotriene B4 receptor antagonist [Herron et al. Leukotriene B4 receptor antagonists: the LY255283 series of hydroxyacetophenones. Journal of Medicinal Chemistry 35:1818-1828 (1992)].

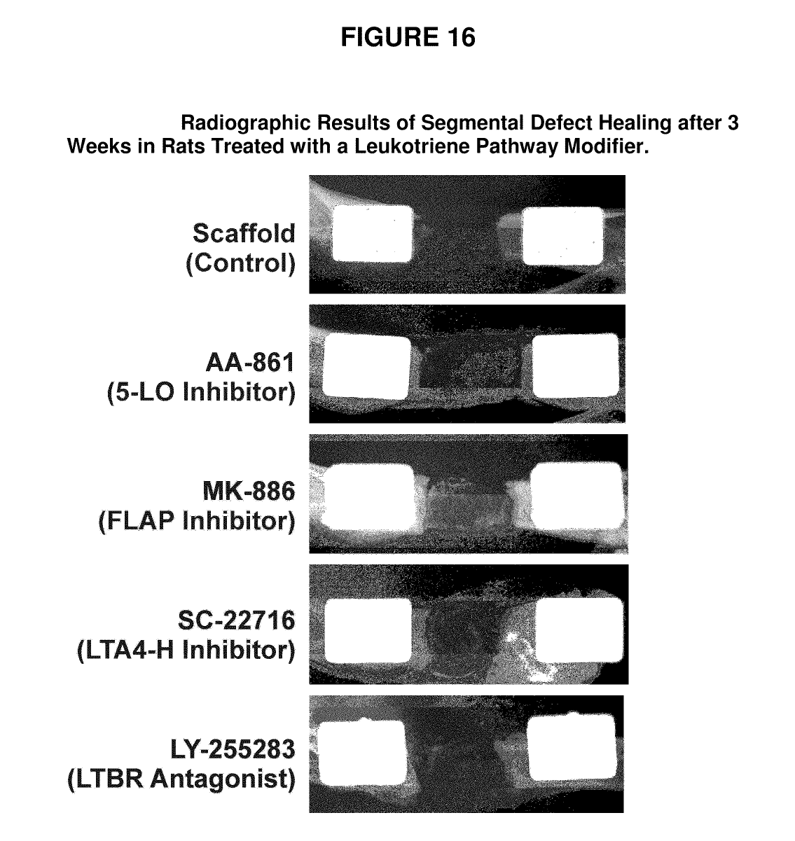

Healing of the segmental defects was assessed after 3 weeks of healing by radiographs (FIG. 16). Visual inspection of radiographs show significantly more bone formation as evident by the x-ray dense material in the segmental defect region in all drug-treatment groups as compared to the scaffold-only group. These data show that local application of a 5-LO inhibitor, a FLAP inhibitor, an LTA4-hydrolase inhibitor, or an LTB4 receptor antagonist promotes osteogenesis and further support the conclusion that 5-LO or LTA4-hydrolase activity or 5-LO or LTA4-hydrolase dependent signaling negatively regulates osteogenesis.

Thus, novel methods for promoting osteogenesis to accelerate or enhance bone fracture healing, treat bone defects, and enhance bone formation are disclosed. Although preferred embodiments of the subject invention have been described in some detail, it is understood that obvious variations can be made without departing from the spirit and the scope of the invention as defined by the appended claims.

1. A method for promoting osteogenesis to treat a mammalian subject in need thereof, comprising: administering to said subject a pharmaceutically effective amount of a compound that reduces a leukotriene B4 activity, wherein said leukotriene B4 activity reduction promotes osteogenesis in said subject.

2. The method of claim 1, wherein said method treats a bone fracture in said subject.

3. The method of claim 2, wherein said bone fracture is a non-osteoporotic fracture, an osteoporotic fracture, a fracture associated with a congenital disease, a fracture associated with an acquired disease, or an osteotomic fracture.

4. The method of claim 3, wherein said bone fracture is a non-osteoporotic fracture.

5. The method of claim 3, wherein said bone fracture is an osteoporotic fracture.

6. The method of claim 3, wherein said bone fracture is an osteotomic fracture.

7. The method of claim 1, wherein said method induces bone formation in said subject.

8. The method of claim 7, wherein said subject is receiving spinal fusion or joint arthrodesis treatment.

9. The method of claim 1, wherein said method treats a bone defect in said subject.

10. The method of claim 1, wherein said compound reduces a leukotriene B4 activity by inhibiting a leukotriene A4 hydrolase activity.

11. The method of claim 1, wherein said compound reduces a leukotriene B4 activity by antagonizing a leukotriene B4 receptor activity.

12. The method of claim 1, wherein said compound comprises a small molecule.

13. The method of claim 12, wherein said small molecule is selected from the group consisting of captopril; bestatin; JNJ-27265732; JNJ-26993135 (1-[4-(benzothiazol-2-yloxy)-benzyl]-piperidine-4-carboxylic acid); SC-57461A (3-[methyl[3-[4-phenylmethyl)phenoxy]propyl]amino]propanoic acid HCl); SC-22716 (1,1-[2-(4-phenylphenoxy)ethyl]pyrrolidine); SC-56938; SA-6541 (S-(4-dimethyl-aminobenzyl)-N-[(2S)-3-mercapto-2-methylpropionyl]-L-cysteine) Santen Pharmaceuticals; SA-9499; RP-64966 Rhône-Poulenc Rorer; LY-293111 [2-[2-propyl-3-[3-[2-ethyl-4-(4-fluorophenyl)-5-hydroxyphenoxy]-propoxy]-phenoxy]benzoic acid]; ONO-4057 (ONO Pharmaceutical); BIIL284; LY-255283 [1-(5-ethyl-2-hydroxy-4-(6-methyl-6-(1H-tetrazol-5-yl)-heptoxy+++)-phenyl)ethanone]; CP-195,543 [(+)-2-(3-benzyl-4-hydroxy-chroman-7-yl)-4-trifluoromethyl-benzoic acid]; SC-41930 [7-[3-(4-acetyl-3-methoxy-2-propylphenoxy)propoxyl]-3,4-dihydro-8-propyl-2H-1-benzopyran-2-carboxylic acid]; SC-53228 [(+)-(S)-7-[3-(2-cyclopropyl-methyl)-3-methoxy-4-[(methylamino) carbonyl]phenoxy]propoxy]-3,4-dihydro-8-propyl-2H-1-benzopyran-2-propanoic acid]; SC-51146; SC-53229; and SC-45694 [7-[4-(1-hydroxy-3Z-nonenyl)phenyl]-5S-hydroxy-6Z-hept enoic acid lithium salt].

14. The method of claim 1, further comprising administering to said subject a pharmaceutically effective amount of a compound that reduces a COX-1 activity.

15. The method of claim 14, wherein said compound is selected from the group consisting of SC-560, FR122047, Valeroyl salicylate, Aspirin, Dexketoprofene, Keterolac, Flurbiprofen, and Suprofen.

16. The method of claim 1, further comprising administering to said subject a pharmaceutically effective amount of a compound that increases a COX-2 activity.

17. The method of claim 16, wherein said compound is selected from the group consisting of Prostaglandin E2, butaprost, sulprostone, CP-536, 745-01, CP-043,305-02, CP-044,519-02, CP432, ONO-4819, CP-533,536, prostaglandin F_{2α}, bimatoprost, cloprostenol, latanoprost, tafluprost, bone morphogenetic protein-2 (BMP2), platelet derived growth factor (PDGF), interleukin-1α, interleukin-1β, tumor necrosis factor-alpha (TNF-α), fibroblast growth factor (FGF), transforming growth factor-β (TGF-β), epidermal growth factor (EGF), parathyroid hormone (PTH), parathyroid hormone related peptide (PTHrP), teriparatide and derivatives, recombinant forms and mimetics of these compounds.

18. The method of claim 1, comprising administering to said subject an ultrasound therapy or exposing said subject to a pulsed electromagnetic field in an amount sufficient to increase a COX-2 activity in said subject.

19. The method of claim 1, wherein said subject is diagnosed with a bone fracture or bone defect prior to administration of said compound.

20.-47. (canceled)

48. A method for promoting osteogenesis to treat a mammalian subject in need thereof, comprising:
obtaining a biological sample;
contacting said biological sample with a pharmaceutically effective amount of compound that reduces a leukotriene B4 activity; and
administering said biological sample to said mammalian subject, wherein said contacted biological sample promotes osteogenesis in said subject.

49.-76. (canceled)


SEQUENCE LISTING (91 sequences)

SEQ ID NO: 1 - DNA, 2568 bp, Homo sapiens
gccagggaccagtggtgggaggaggctgcggcgctagatgcggacacctggaccgccgcgccgaggctcccggcgctcgctgctcccgcggcccgcgccatgccctcctacacggtcaccgtggccactggcagccagtggttcgccggcactgacgactacatctacctcagcctcgtgggctcggcgggctgcagcgagaagcacctgctggacaagcccttctacaacgacttcgagcgtggcgcggtggattcatacgacgtgactgtggacgaggaactgggcgagatccagctggtcagaatcgagaagcgcaagtactggctgaatgacgactggtacctgaagtacatcacgctgaagacgccccacggggactacatcgagttcccctgctaccgctggatcaccggcgatgtcgaggttgtcctgagggatggacgcgcaaagttggcccgagatgaccaaattcacattctcaagcaacaccgacgtaaagaactggaaacacggcaaaaacaatatcgatggatggagtggaaccctggcttccccttgagcatcgatgccaaatgccacaaggatttaccccgtgatatccagtttgatagtgaaaaaggagtggactttgttctgaattactccaaagcgatggagaacctgttcatcaaccgcttcatgcacatgttccagtcttcttggaatgacttcgccgactttgagaaaatctttgtcaagatcagcaacactatttctgagcgggtcatgaatcactggcaggaagacctgatgtttggctaccagttcctgaatggctgcaaccctgtgttgatccggcgctgcacagagctgcccgagaagctcccggtgaccacggagatggtagagtgcagcctggagcggcagctcagcttggagcaggaggtccagcaagggaacattttcatcgtggactttgagctgctggatggcatcgatgccaacaaaacagacccctgcacactccagttcctggccgctcccatctgcttgctgtataagaacctggccaacaagattgtccccattgccatccagctcaaccaaatcccgggagatgagaaccctattttcctcccttcggatgcaaaatacgactggcttttggccaaaatctgggtgcgttccagtgacttccacgtccaccagaccatcacccaccttctgcgaacacatctggtgtctgaggtttttggcattgcaatgtaccgccagctgcctgctgtgcaccccattttcaagctgctggtggcacacgtgagattcaccattgcaatcaacaccaaggcccgtgagcagctcatctgcgagtgtggcctctttgacaaggccaacgccacagggggcggtgggcacgtgcagatggtgcagagggccatgaaggacctgacctatgcctccctgtgctttcccgaggccatcaaggcccggggcatggagagcaaagaagacatcccctactacttctaccgggacgacgggctcctggtgtgggaagccatcaggacgttcacggccgaggtggtagacatctactacgagggcgaccaggtggtggaggaggacccggagctgcaggacttcgtgaacgatgtctacgtgtacggcatgcggggccgcaagtcctcaggcttccccaagtcggtcaagagccgggagcagctgtcggagtacctgaccgtggtgatcttcaccgcctccgcccagcacgccgcggtcaacttcggccagtacgactggtgctcctggatccccaatgcgcccccaaccatgcgagccccgccaccgactgccaagggcgtggtgaccattgagcagatcgtggacacgctgcccgaccgcggccgctcctgctggcatctgggtgcagtgtgggcgctgagccagttccaggaaaacgagctgttcctgggcatgtacccagaagagcattttatcgagaagcctgtgaaggaagccatggcccgattccgcaagaacctcgaggccattgtcagcgtgattgctgagcgcaacaagaagaagcagctgccatattactacttgtccccagaccggattccgaacagtgtggccatctgagcacactgccagtctcactgtgggaaggccagctgccccagccagatggactccagcctgcctggcaggctgtctggccaggcctcttggcagtcacatctcttcctccgaggccagtacctttccatttattctttgatcttcagggaactgcatagattgatcaaagtgtaaacaccatagggacccattctacacagagcaggactgcacagcgtcctgtccacacccagctcagcatttccacaccaagcagcaacagcaaatcacgaccactgatagatgtctattcttgttggagacatgggatgattattttctgttctatttgtgcttagtccaattccttgcacatagtaggtacccaattcaattactattgaatgaattaagaattggttgccataaaaataaatcagttcatttaaaaaaaaaaaaaaaaaa

SEQ ID NO: 2 - protein, 674 aa, Homo sapiens
MPSYTVTVATGSQWFAGTDDYIYLSLVGSAGCSEKHLLDKPFYNDFERGAVDSYDVTVDEELGEIQLVRIEKRKYWLNDDWYLKYITLKTPHGDYIEFPCYRWITGDVEVVLRDGRAKLARDDQIHILKQHRRKELETRQKQYRWMEWNPGFPLSIDAKCHKDLPRDIQFDSEKGVDFVLNYSKAMENLFINRFMHMFQSSWNDFADFEKIFVKISNTISERVMNHWQEDLMFGYQFLNGCNPVLIRRCTELPEKLPVTTEMVECSLERQLSLEQEVQQGNIFIVDFELLDGIDANKTDPCTLQFLAAPICLLYKNLANKIVPIAIQLNQIPGDENPIFLPSDAKYDWLLAKIWVRSSDFHVHQTITHLLRTHLVSEVFGIAMYRQLPAVHPIFKLLVAHVRFTIAINTKAREQLICECGLFDKANATGGGGHVQMVQRAMKDLTYASLCFPEAIKARGMESKEDIPYYFYRDDGLLVWEAIRTFTAEVVDIYYEGDQVVEEDPELQDFVNDVYVYGMRGRKSSGFPKSVKSREQLSEYLTVVIFTASAQHAAVNFGQYDWCSWIPNAPPTMRAPPPTAKGVVTIEQIVDTLPDRGRSCWHLGAVWALSQFQENELFLGMYPEEHFIEKPVKEAMARFRKNLEAIVSVIAERNKKKQLPYYYLSPDRIPNSVAI

SEQ ID NO: 3 - DNA, 873 bp, Homo sapiens
acttccccttcctgtacagggcaggttgtgcagctggaggcagagcagtcctctctggggagcctgaagcaaacatggatcaagaaactgtaggcaatgttgtcctgttggccatcgtcaccctcatcagcgtggtccagaatggattctttgcccataaagtggagcacgaaagcaggacccagaatgggaggagcttccagaggaccggaacacttgcctttgagcgggtctacactgccaaccagaactgtgtagatgcgtaccccactttcctcgctgtgctctggtctgcggggctactttgcagccaagttcctgctgcgtttgctggactgatgtacttgtttgtgaggcaaaagtactttgtcggttacctaggagagagaacgcagagcacccctggctacatatttgggaaacgcatcatactcttcctgttcctcatgtccgttgctggcatattcaactattacctcatcttctttttcggaagtgactttgaaaactacataaagacgatctccaccaccatctcccctctacttctcattccctaactctctgctgaatatggggttggtgttctcatctaatcaatacctacaagtcatcataattcagctcttgagagcattctgctcttctttagatggctgtaaatctattggccatctgggcttcacagcttgagttaaccttgcttttccgggaacaaaatgatgtcatgtcagctccgccccttgaacatgaccgtggccccaaatttgctattcccatgcattttgtttgtttcttcacttatcctgttctctgaagatgttttgtgaccaggtttgtgttttcttaaaataaaatgcagagacatgtttt

SEQ ID NO: 4 - protein, 161 aa, Homo sapiens
MDQETVGNVVLLAIVTLISVVQNGFFAHKVEHESRTQNGRSFQRTGTLAFERVYTANQNCVDAYPTFLAVLWSAGLLCSQVPAAFAGLMYLFVRQKYFVGYLGERTQSTPGYIFGKRIILFLFLMSVAGIFNYYLIFFFGSDFENYIKTISTTISPLLLIP

SEQ ID NO: 5 - DNA, 4465 bp, Homo sapiens
caattgtcatacgacttgcagtgagcgtcaggagcacgtccaggaactcctcagcagcgcctccttcagctccacagccagacgccctcagacagcaaagcctacccccgcgccgcgccctgcccgccgctcggatgctcgcccgcgccctgctgctgtgcgcggtcctggcgctcagccatacagcaaatccttgctgttcccacccatgtcaaaaccgaggtgtatgtatgagtgtgggatttgaccagtataagtgcgattgtacccggacaggattctatggagaaaactgctcaacaccggaatttttgacaagaataaaattatttctgaaacccactccaaacacagtgcactacatacttacccacttcaagggattttggaacgttgtgaataacattcccttccttcgaaatgcaattatgagttatgtcttgacatccagatcacatttgattgacagtccaccaacttacaatgctgactatggctacaaaagctgggaagccttctctaacctctcctattatactagagcccttcctcctgtgcctgatgattgcccgactcccttgggtgtcaaaggtaaaaagcagcttcctgattcaaatgagattgtggaaaaattgcttctaagaagaaagttcatccctgatccccagggctcaaacatgatgtttgcattctttgcccagcacttcacgcatcagtttttcaagacagatcataagcgagggccagctttcaccaacgggctgggccatggggtggacttaaatcatatttacggtgaaactctggctagacagcgtaaactgcgccttttcaaggatggaaaaatgaaatatcagataattgatggagagatgtatcctcccacagtcaaagatactcaggcagagatgatctaccctcctcaagtccctgagcatctacggtttgctgtggggcaggaggtctttggtctggtgcctggtctgatgatgtatgccacaatctggctgcgggaacacaacagagtatgcgatgtgcttaaacaggagcatcctgaatggggtgatgagcagttgttccagacaagcaggctaatactgataggagagactattaagattgtgattgaagattatgtgcaacacttgagtggctatcacttcaaactgaaatttgacccagaactacttttcaacaaacaattccagtaccaaaatcgtattgctgctgaatttaacaccctctatcactggcatccccttctgcctgacacctttcaaattcatgaccagaaatacaactatcaacagtttatctacaacaactctatattgctggaacatggaattacccagtttgttgaatcattcaccaggcaaattgctggcagggttgctggtggtaggaatgttccacccgcagtacagaaagtatcacaggcttccattgaccagagcaggcagatgaaataccagtcttttaatgagtaccgcaaacgctttatgctgaagccctatgaatcatttgaagaacttacaggagaaaaggaaatgtctgcagagttggaagcactctatggtgacatcgatgctgtggagctgtatcctgcccttctggtagaaaagcctcggccagatgccatctttggtgaaaccatggtagaagttggagcaccattctccttgaaaggacttatgggtaatgttatatgttctcctgcctactggaagccaagcacttttggtggagaagtgggttttcaaatcatcaacactgcctcaattcagtctctcatctgcaataacgtgaagggctgtccctttacttcattcagtgttccagatccagagctcattaaaacagtcaccatcaatgcaagttcttcccgctccggactagatgatatcaatcccacagtactactaaaagaacgttcgactgaactgtagaagtctaatgatcatatttatttatttatatgaaccatgtctattaatttaattatttaataatatttatattaaactccttatgttacttaacatcttctgtaacagaagtcagtactcctgttgcggagaaaggagtcatacttgtgaagacttttatgtcactactctaaagattttgctgttgctgttaagtttggaaaacagtttttattctgttttataaaccagagagaaatgagttttgacgtctttttacttgaatttcaacttatattataagaacgaaagtaaagatgtttgaatacttaaacactatcacaagatggcaaaatgctgaaagtttttacactgtcgatgtttccaatgcatcttccatgatgcattagaagtaactaatgtttgaaattttaaagtacttttggttatttttctgtcatcaaacaaaaacaggtatcagtgcattattaaatgaatatttaaattagacattaccagtaatttcatgtctactttttaaaatcagcaatgaaacaataatttgaaatttctaaattcatagggtagaatcacctgtaaaagcttgtttgatttcttaaagttattaaacttgtacatataccaaaaagaagctgtcttggatttaaatctgtaaaatcagatgaaattttactacaattgcttgttaaaatattttataagtgatgttcctttttcaccaagagtataaacctttttagtgtgactgttaaaacttccttttaaatcaaaatgccaaatttattaaggtggtggagccactgcagtgttatctcaaaataagaatattttgttgagatattccagaatttgtttatatggctggtaacatgtaaaatctatatcagcaaaagggtctacctttaaaataagcaataacaaagaagaaaaccaaattattgttcaaatttaggtttaaacttttgaagcaaacttttttttatccttgtgcactgcaggcctggtactcagattttgctatgaggttaatgaagtaccaagctgtgcttgaataacgatatgttttctcagattttctgttgtacagtttaatttagcagtccatatcacattgcaaaagtagcaatgacctcataaaatacctcttcaaaatgcttaaattcatttcacacattaattttatctcagtcttgaagccaattcagtaggtgcattggaatcaagcctggctacctgcatgctgttccttttcttttcttcttttagccattttgctaagagacacagtcttctcatcacttcgtttctcctattttgttttactagttttaagatcagagttcactttctttggactctgcctatattttcttacctgaacttttgcaagttttcaggtaaacctcagctcaggactgctatttagctcctcttaagaagattaaaagagaaaaaaaaaggcccttttaaaaatagtatacacttattttaagtgaaaagcagagaattttatttatagctaattttagctatctgtaaccaagatggatgcaaagaggctagtgcctcagagagaactgtacggggtttgtgactggaaaaagttacgttcccattctaattaatgccctttcttatttaaaaacaaaaccaaatgatatctaagtagttctcagcaataataataatgacgataatacttcttttccacatctcattgtcactgacatttaatggtactgtatattacttaatttattgaagattattatttatgtcttattaggacactatggttataaactgtgtttaagcctacaatcattgatttttttttgttatgtcacaatcagtatattttctttggggttacctctctgaatattatgtaaacaatccaaagaaatgattgtattaagatttgtgaataaatttttagaaatctgattggcatattgagatatttaaggttgaatgtttgtccttaggataggcctatgtgctagcccacaaagaatattgtctcattagcctgaatgtgccataagactgaccttttaaaatgttttgagggatctgtggatgcttcgttaatttgttcagccacaatttattgagaaaatattctgtgtcaagcactgtgggttttaatatttttaaatcaaacgctgattacagataatagtatttatataaataattgaaaaaaattttcttttgggaagagggagaaaatgaaataaatatcattaaagataactcaggagaatcttctttacaattttacgtttagaatgtttaaggttaagaaagaaatagtcaatatgcttgtataaaacactgttcactgttttttttaaaaaaaaaacttgatttgttattaacattgatctgctgacaaaacctgggaatttgggttgtgtatgcgaatgtttcagtgcctcagacaaatgtgtatttaacttatgtaaaagataagtctggaaataaatgtctgtttatttttgtactattta

SEQ ID NO: 6 - protein, 604 aa, Homo sapiens
MLARALLLCAVLALSHTANPCCSHPCQNRGVCMSVGFDQYKCDCTRTGFYGENCSTPEFLTRIKLFLKPTPNTVHYILTHFKGFWNVVNNIPFLRNAIMSYVLTSRSHLIDSPPTYNADYGYKSWEAFSNLSYYTRALPPVPDDCPTPLGVKGKKQLPDSNEIVEKLLLRRKFIPDPQGSNMMFAFFAQHFTHQFFKTDHKRGPAFTNGLGHGVDLNHIYGETLARQRKLRLFKDGKMKYQIIDGEMYPPTVKDTQAEMIYPPQVPEHLRFAVGQEVFGLVPGLMMYATIWLREHNRVCDVLKQEHPEWGDEQLFQTSRLILIGETIKIVIEDYVQHLSGYHFKLKFDPELLFNKQFQYQNRIAAEFNTLYHWHPLLPDTFQIHDQKYNYQQFIYNNSILLEHGITQFVESFTRQIAGRVAGGRNVPPAVQKVSQASIDQSRQMKYQSFNEYRKRFMLKPYESFEELTGEKEMSAELEALYGDIDAVELYPALLVEKPRPDAIFGETMVEVGAPFSLKGLMGNVICSPAYWKPSTFGGEVGFQIINTASIQSLICNNVKGCPFTSFSVPDPELIKTVTINASSSRSGLDDINPTVLLKERSTEL

SEQ ID NO: 7 - DNA, 5093 bp, Homo sapiens
aggtgacagctggagggaggagcgggggtggagccgggggaagggtggggaggggatgggctggagctccgggcagtgtgcgaggcgcacgcacaggagcctgcactctgcgtcccgcaccccagcagccgcgccatgagccggagtctcttgctctggttcttgctgttcctgctcctgctcccgccgctccccgtcctgctcgcggacccaggggcgcccacgccagtgaatccctgttgttactatccatgccagcaccagggcatctgtgtccgcttcggccttgaccgctaccagtgtgactgcacccgcacgggctattccggccccaactgcaccatccctggcctgtggacctggctccggaattcactgcggcccagcccctctttcacccacttcctgctcactcacgggcgctggttctgggagtttgtcaatgccaccttcatccgagagatgctcatgcgcctggtactcacagtgcgctccaaccttatccccagtccccccacctacaactcagcacatgactacatcagctgggagtctttctccaacgtgagctattacactcgtattctgccctctgtgcctaaagattgccccacacccatgggaaccaaagggaagaagcagttgccagatgcccagctcctggcccgccgcttcctgctcaggaggaagttcatacctgacccccaaggcaccaacctcatgtttgccttctttgcacaacacttcacccaccagttcttcaaaacttctggcaagatgggtcctggcttcaccaaggccttgggccatggggtagacctcggccacatttatggagacaatctggagcgtcagtatcaactgcggctctttaaggatgggaaactcaagtaccaggtgctggatggagaaatgtacccgccctcggtagaagaggcgcctgtgttgatgcactacccccgaggcatcccgccccagagccagatggctgtgggccaggaggtgtttgggctgcttcctgggctcatgctgtatgccacgctctggctacgtgagcacaaccgtgtgtgtgacctgctgaaggctgagcaccccacctggggcgatgagcagcttttccagacgacccgcctcatcctcataggggagaccatcaagattgtcatcgaggagtacgtgcagcagctgagtggctatttcctgcagctgaaatttgacccagagctgctgttcggtgtccagttccaataccgcaaccgcattgccatggagttcaaccatctctaccactggcaccccctcatgcctgactccttcaaggtgggctcccaggagtacagctacgagcagttcttgttcaacacctccatgttggtggactatggggttgaggccctggtggatgccttctctcgccagattgctggccggatcggtgggggcaggaacatggaccaccacatcctgcatgtggctgtggatgtcatcagggagtctcgggagatgcggctgcagcccttcaatgagtaccgcaagaggtttggcatgaaaccctacacctccttccaggagctcgtaggagagaaggagatggcagcagagttggaggaattgtatggagacattgatgcgttggagttctaccctggactgcttcttgaaaagtgccatccaaactctatctttggggagagtatgatagagattggggctcccttttccctcaagggtctcctagggaatcccatctgttctccggagtactggaagccgagcacatttggcggcgaggtgggctttaacattgtcaagacggccacactgaagaagctggtctgcctcaacaccaagacctgtccctacgtttccttccgtgtgccggatgccagtcaggatgatgggcctgctgtggagcgaccatccacagagctctgaggggcaggaaagcagcattctggaggggagagctttgtgcttgtcattccagagtgctgaggccagggctgatggtcttaaatgctcattttctggtttggcatggtgagtgttggggttgacatttagaactttaagtctcacccattatctggaatattgtgattctgtttattcttccagaatgctgaactccttgttagcccttcagattgttaggagtggttctcatttggtctgccagaatactgggttcttagttgacaacctagaatgtcagatttctggttgatttgtaacacagtcattctaggatgtggagctactgatgaaatctgctagaaagttagggggttcttattttgcattccagaatcttgactttctgattggtgattcaaagtgttgtgttcctggctgatgatccagaacagtggctcgtatcccaaatctgtcagcatctggctgtctagaatgtggatttgattcattttcctgttcagtgagatatcatagagacggagatcctaaggtccaacaagaatgcattccctgaatctgtgcctgcactgagagggcaaggaagtggggtgttcttcttgggacccccactaagaccctggtctgaggatgtagagagaacaggtgggctgtattcacgccattggttggaagctaccagagctctatccccatccaggtcttgactcatggcagctgtttctcatgaagctaataaaattcgctttctaaagttacctgttatatatctcttttggtcccatcctctaaagcagaggcaacactggaacatggctagcctttcttgtagccatggctgggcgtgctagaggttgcagcatgagactttctgctgggatccttgggcccatcactgtatagacatgctaccactggtacttcctttctccctgcgggccaggcactgcccttttcaggaagctctcttaaaatacccattgccccagacctggaagatataacattcagttcccaccatctgattaaaacaacttcctcccttacagagcatacaacagagggggcacccggggaggagagcacatactgtgttccaatttcacgcttttaattctcatttgttctcacaccaacagtgtgaagtgcgtggtataatctccatttcaaaaccaaggaagcagcctcagagtggtcgagtgacacacctcacgcaggctgagtccagagcttgtgctcctcttgattcctggtttgactcagttccaggcctgatcttgcctgtctggctcagggtcaaagacagaatggtggagtgtagcctccacctgatattcaggctactcattcagtcccaaatatgtattttcctaagtgtttactatgtgccagttcctgtaacaggtgtggggacacagcagtgagtaatcaatacagacaaggttctgcccttatggagctcacactccagtggcagacaaacagaccataaataaggaaacgatgaaataagatatatacaaggtgagtgtgacttcccttctaaccccctctgctctgtcctcccctattgcgctctcaagaccagagacccaacagcagtgatctcagggcagacagccctccactccagctctgagacccttttctcaggacctctgtaggcagcagagagagaggacagaggggtaagatgaggggttgagggaaggttcttcatgatccacactttgggcttagtatttctcaggaagagctatggcccagaaacaacaggggaaactagagttcggtctgacagtccttggggttaagtctcctgtcttatggtccagaaactcctgtttctccttagttggctggaaactgctcccatcattccttctggcctctgctgaatgcagggaatgcaatccttccctgctcttgcagttgctctgacgtagaaagatccttcgggtgctggaagtctccatgaagagcttgtgtcctgtcctttcttgcagattctatttcccctcttctgctaatacctcttactttgcttgagaatcctctcctttcttattaatttcagtcttggtggttctatcaggggtgcattctggccaaggggtgggcctgtgaatcaatcctgggcaatcagacaccctctccttaaaaactggcccgtggagactgagatcactgactctgactcatccccacagctggctctgacaagatggtccatttgttcctgcttccgagatccccagggcagcctggatccctgcccttctcaagactttagcttttccttccatccggtggcctattccaggaattcctcttttgcttaaatcagttggagtttgtgtctgttgcttgtaatcaagcctttatggctgctgggctgagtgacacaagcactttaatggcctggagggacttttaatcagtgaagatgcaatcagacaagtgttttggaaagagcaccctcgagaagggtggatgacagggcagagcaggaaggacaggaagctggcagaacggaggaggctgcagccgtggtccaaccaggagctgatggcagctggggctaggggaagggctttgagggtggaaggatgggatgggttccagaggtattcctctcttaaatgcaagtgcctagattaggtagactttgcttagtattgacaactgcacatgaaagttttgcaaagggaaacaggctaaatgcaccaagaaagcttcttcagagtgaagaatcttaatgcttgtaatttaaacatttgttcctggagttttgatttggtggatgtgatggttggttttatttgtcagtttggttgggctatagcacacagttatttaatcaaacagtaatctaggtgtggctgtgaaggtattttgtagatgtgattaacatctacaatcagttgactttaagtgaaagagattacttaaataatttgggtgagctgcacctgattagttgaaaggcctcaagaacaaacactgcagtttcctggaaaagaagaaactttgcctcaagactatagccatcgactcctgcctgagtttccagcctgctagtctgccctatggatttgaagtttgccaaccccaacaattgtgtgaattaatttctaaaaataaagctatatacagcc

SEQ ID NO: 8 - protein, 599 aa, Homo sapiens
MSRSLLLWFLLFLLLLPPLPVLLADPGAPTPVNPCCYYPCQHQGICVRFGLDRYQCDCTRTGYSGPNCTIPGLWTWLRNSLRPSPSFTHFLLTHGRWFWEFVNATFIREMLMRLVLTVRSNLIPSPPTYNSAHDYISWESFSNVSYYTRILPSVPKDCPTPMGTKGKKQLPDAQLLARRFLLRRKFIPDPQGTNLMFAFFAQHFTHQFFKTSGKMGPGFTKALGHGVDLGHIYGDNLERQYQLRLFKDGKLKYQVLDGEMYPPSVEEAPVLMHYPRGIPPQSQMAVGQEVFGLLPGLMLYATLWLREHNRVCDLLKAEHPTWGDEQLFQTTRLILIGETIKIVIEEYVQQLSGYFLQLKFDPELLFGVQFQYRNRIAMEFNHLYHWHPLMPDSFKVGSQEYSYEQFLFNTSMLVDYGVEALVDAFSRQIAGRIGGGRNMDHHILHVAVDVIRESREMRLQPFNEYRKRFGMKPYTSFQELVGEKEMAAELEELYGDIDALEFYPGLLLEKCHPNSIFGESMIEIGAPFSLKGLLGNPICSPEYWKPSTFGGEVGFNIVKTATLKKLVCLNTKTCPYVSFRVPDASQDDGPAVERPSTEL

SEQ ID NO: 9 - DNA, 21 bp, Artificial Sequence
  Description of Artificial Sequence Synthetic oligonucleotide
aactgggcgagatccagctgg

SEQ ID NO: 10 - DNA, 21 bp, Artificial Sequence
  Description of Artificial Sequence Synthetic oligonucleotide
aagctcccggtgaccacggag

SEQ ID NO: 11 - DNA, 21 bp, Artificial Sequence
  Description of Artificial Sequence Synthetic oligonucleotide
aaggaagccatggcccgattc

SEQ ID NO: 12 - DNA, 21 bp, Artificial Sequence
  Description of Artificial Sequence Synthetic oligonucleotide
aatcgagaagcgcaagtactg

SEQ ID NO: 13 - DNA, 21 bp, Artificial Sequence
  Description of Artificial Sequence Synthetic oligonucleotide
aaggagtggactttgttctga

SEQ ID NO: 14 - DNA, 21 bp, Artificial Sequence
  Description of Artificial Sequence Synthetic oligonucleotide
aacttcggccagtacgactgg

SEQ ID NO: 15 - DNA, 21 bp, Artificial Sequence
  Description of Artificial Sequence Synthetic oligonucleotide
aagttggcccgagatgaccaa

SEQ ID NO: 16 - DNA, 21 bp, Artificial Sequence
  Description of Artificial Sequence Synthetic oligonucleotide
aacacatctggtgtctgaggt

SEQ ID NO: 17 - DNA, 21 bp, Artificial Sequence
  Description of Artificial Sequence Synthetic oligonucleotide
aaccatgcgagccccgccacc

SEQ ID NO: 18 - DNA, 21 bp, Artificial Sequence
  Description of Artificial Sequence Synthetic oligonucleotide
aagcaaacatggatcaagaaa

SEQ ID NO: 19 - DNA, 21 bp, Artificial Sequence
  Description of Artificial Sequence Synthetic oligonucleotide
aagttcctgctgcgtttgctg

SEQ ID NO: 20 - DNA, 21 bp, Artificial Sequence
  Description of Artificial Sequence Synthetic oligonucleotide
aattcagctcttgagagcatt

SEQ ID NO: 21 - DNA, 21 bp, Artificial Sequence
  Description of Artificial Sequence Synthetic oligonucleotide
aatggattctttgcccataaa

SEQ ID NO: 22 - DNA, 21 bp, Artificial Sequence
  Description of Artificial Sequence Synthetic oligonucleotide
aagtactttgtcggttaccta

SEQ ID NO: 23 - DNA, 21 bp, Artificial Sequence
  Description of Artificial Sequence Synthetic oligonucleotide
aatctattggccatctgggct

SEQ ID NO: 24 - DNA, 21 bp, Artificial Sequence
  Description of Artificial Sequence Synthetic oligonucleotide
aaccagaactgtgtagatgcg

SEQ ID NO: 25 - DNA, 21 bp, Artificial Sequence
  Description of Artificial Sequence Synthetic oligonucleotide
aagtgactttgaaaactacat

SEQ ID NO: 26 - DNA, 21 bp, Artificial Sequence
  Description of Artificial Sequence Synthetic oligonucleotide
aatgatgtcatgtcagctccg

SEQ ID NO: 27 - DNA, 19 bp, Artificial Sequence
  Description of Artificial Sequence Synthetic oligonucleotide
agaaagagcaggtggaaaa

SEQ ID NO: 28 - DNA, 19 bp, Artificial Sequence
  Description of Artificial Sequence Synthetic oligonucleotide
caaatatgctcttggagaa

SEQ ID NO: 29 - DNA, 19 bp, Artificial Sequence
  Description of Artificial Sequence Synthetic oligonucleotide
ggacactccttctgtgaaa

SEQ ID NO: 30 - DNA, 19 bp, Artificial Sequence
  Description of Artificial Sequence Synthetic oligonucleotide
ctaaagaactggtggcact

SEQ ID NO: 31 - DNA, 19 bp, Artificial Sequence
  Description of Artificial Sequence Synthetic oligonucleotide
tgacaaatcccatgatcaa

SEQ ID NO: 32 - DNA, 19 bp, Artificial Sequence
  Description of Artificial Sequence Synthetic oligonucleotide
ggagaaagacaaagttaca

SEQ ID NO: 33 - DNA, 19 bp, Artificial Sequence
  Description of Artificial Sequence Synthetic oligonucleotide
agatatagaccctgatgta

SEQ ID NO: 34 - DNA, 19 bp, Artificial Sequence
  Description of Artificial Sequence Synthetic oligonucleotide
gcttggaggaccagagatt

SEQ ID NO: 35 - DNA, 19 bp, Artificial Sequence
  Description of Artificial Sequence Synthetic oligonucleotide
ccacagacctgaaggatct

SEQ ID NO: 36 - DNA, 19 bp, Artificial Sequence
  Description of Artificial Sequence Synthetic oligonucleotide
ggacatagggcgtcggcta

SEQ ID NO: 37 - DNA, 19 bp, Artificial Sequence
  Description of Artificial Sequence Synthetic oligonucleotide
ggttagggctcgtggggaa

SEQ ID NO: 38 - DNA, 19 bp, Artificial Sequence
  Description of Artificial Sequence Synthetic oligonucleotide
ggacagtagtgccctggaa

SEQ ID NO: 39 - DNA, 19 bp, Artificial Sequence
  Description of Artificial Sequence Synthetic oligonucleotide
gctttgtggtgtggagtat

SEQ ID NO: 40 - DNA, 19 bp, Artificial Sequence
  Description of Artificial Sequence Synthetic oligonucleotide
ggtgtggagtatcctgaaa

SEQ ID NO: 41 - DNA, 19 bp, Artificial Sequence
  Description of Artificial Sequence Synthetic oligonucleotide
gcccaaggcacctggagtt

SEQ ID NO: 42 - DNA, 19 bp, Artificial Sequence
  Description of Artificial Sequence Synthetic oligonucleotide
ctcactaggtgtagagttc

SEQ ID NO: 43 - DNA, 19 bp, Artificial Sequence
  Description of Artificial Sequence Synthetic oligonucleotide
gcatctgggtgttgtcctt

SEQ ID NO: 44 - DNA, 19 bp, Artificial Sequence
  Description of Artificial Sequence Synthetic oligonucleotide
gcgtgaaccccgtgctgta

SEQ ID NO: 45 - DNA, 19 bp, Artificial Sequence
  Description of Artificial Sequence Synthetic oligonucleotide
gctgcaaggcggtgtacta

SEQ ID NO: 46 - DNA, 19 bp, Artificial Sequence
  Description of Artificial Sequence Synthetic oligonucleotide
ctgctgagctggaagactt

SEQ ID NO: 47 - DNA, 19 bp, Artificial Sequence
  Description of Artificial Sequence Synthetic oligonucleotide
gcgtcaacccggtgctcta

SEQ ID NO: 48 - DNA, 19 bp, Artificial Sequence
  Description of Artificial Sequence Synthetic oligonucleotide
gaaggatgtcggtctgcta

SEQ ID NO: 49 - DNA, 19 bp, Artificial Sequence
  Description of Artificial Sequence Synthetic oligonucleotide
gggaagggaccatggagct

SEQ ID NO: 50 - DNA, 19 bp, Artificial Sequence
  Description of Artificial Sequence Synthetic oligonucleotide
gcgtatgccagctgtgcca

SEQ ID NO: 51 - DNA, 19 bp, Artificial Sequence
  Description of Artificial Sequence Synthetic oligonucleotide
ggggtgggatggagaagga

SEQ ID NO: 52 - DNA, 19 bp, Artificial Sequence
  Description of Artificial Sequence Synthetic oligonucleotide
gcaccttctcatcgggcat

SEQ ID NO: 53 - DNA, 19 bp, Artificial Sequence
  Description of Artificial Sequence Synthetic oligonucleotide
cgtcttcaccgctggagat

SEQ ID NO: 54 - DNA, 22 bp, Artificial Sequence
  Description of Artificial Sequence Synthetic oligonucleotide
gcaggtgcttctcgctgcagcc

SEQ ID NO: 55 - DNA, 20 bp, Artificial Sequence
  Description of Artificial Sequence Synthetic oligonucleotide
gccagtacttgcgcttctcg

SEQ ID NO: 56 - DNA, 20 bp, Artificial Sequence
  Description of Artificial Sequence Synthetic oligonucleotide
ccatcgatattgtttttgcc

SEQ ID NO: 57 - DNA, 24 bp, Artificial Sequence
  Description of Artificial Sequence Synthetic oligonucleotide
ggagcttctcgggcagctctgtgc

SEQ ID NO: 58 - DNA, 21 bp, Artificial Sequence
  Description of Artificial Sequence Synthetic oligonucleotide
ccaggttcttatacagcaagc

SEQ ID NO: 59 - DNA, 23 bp, Artificial Sequence
  Description of Artificial Sequence Synthetic oligonucleotide
ccagcagcttgaaaatggggtgc

SEQ ID NO: 60 - DNA, 19 bp, Artificial Sequence
  Description of Artificial Sequence Synthetic oligonucleotide
gccccgggccttgatggcc

SEQ ID NO: 61 - DNA, 19 bp, Artificial Sequence
  Description of Artificial Sequence Synthetic oligonucleotide
ccacgcccttggcagtcgg

SEQ ID NO: 62 - DNA, 22 bp, Artificial Sequence
  Description of Artificial Sequence Synthetic oligonucleotide
gcggaatcgggccatggcttcc

SEQ ID NO: 63 - DNA, 22 bp, Artificial Sequence
  Description of Artificial Sequence Synthetic oligonucleotide
gttccggtcctctggaagctcc

SEQ ID NO: 64 - DNA, 19 bp, Artificial Sequence
  Description of Artificial Sequence Synthetic oligonucleotide
cgcagaccagagcacagcg

SEQ ID NO: 65 - DNA, 17 bp, Artificial Sequence
  Description of Artificial Sequence Synthetic oligonucleotide
gcaaacgcagcaggaac

SEQ ID NO: 66 - DNA, 20 bp, Artificial Sequence
  Description of Artificial Sequence Synthetic oligonucleotide
cgtttcccaaatatgtagcc

SEQ ID NO: 67 - DNA, 19 bp, Artificial Sequence
  Description of Artificial Sequence Synthetic oligonucleotide
gttttcaaagtcacttccg

SEQ ID NO: 68 - DNA, 21 bp, Artificial Sequence
  Description of Artificial Sequence Synthetic oligonucleotide
ggttaactcaagctgtgaagc

SEQ ID NO: 69 - DNA, 17 bp, Artificial Sequence
  Description of Artificial Sequence Synthetic oligonucleotide
ggagctgacatgacatc

SEQ ID NO: 70 - DNA, 20 bp, Artificial Sequence
  Description of Artificial Sequence Synthetic oligonucleotide
ggccacggtcatgttcaagg

SEQ ID NO: 71 - DNA, 20 bp, Artificial Sequence
  Description of Artificial Sequence Synthetic oligonucleotide
ctgcttgggtcttctgggtc

SEQ ID NO: 72 - DNA, 21 bp, Artificial Sequence
  Description of Artificial Sequence Synthetic oligonucleotide
ctgcttgggtcttctgggtca

SEQ ID NO: 73 - DNA, 20 bp, Artificial Sequence
  Description of Artificial Sequence Synthetic oligonucleotide
cctgcttgggtcttctgggt

SEQ ID NO: 74 - DNA, 20 bp, Artificial Sequence
  Description of Artificial Sequence Synthetic oligonucleotide
tttccacctgctctttctca

SEQ ID NO: 75 - DNA, 19 bp, Artificial Sequence
  Description of Artificial Sequence Synthetic oligonucleotide
gcttgggtcttctgggtca

SEQ ID NO: 76 - DNA, 21 bp, Artificial Sequence
  Description of Artificial Sequence Synthetic oligonucleotide
ctctccctcttcttccactcc

SEQ ID NO: 77 - DNA, 21 bp, Artificial Sequence
  Description of Artificial Sequence Synthetic oligonucleotide
cctctccctcttcttccactc

SEQ ID NO: 78 - DNA, 20 bp, Artificial Sequence
  Description of Artificial Sequence Synthetic oligonucleotide
tctccctcttcttccactcc

SEQ ID NO: 79 - DNA, 21 bp, Artificial Sequence
  Description of Artificial Sequence Synthetic oligonucleotide
tctccctcttcttccactcca

SEQ ID NO: 80 - DNA, 21 bp, Artificial Sequence
  Description of Artificial Sequence Synthetic oligonucleotide
tccacctctccctcttcttcc

SEQ ID NO: 81 - DNA, 20 bp, Artificial Sequence
  Description of Artificial Sequence Synthetic oligonucleotide
tcctacctcctcccacctct

SEQ ID NO: 82 - DNA, 21 bp, Artificial Sequence
  Description of Artificial Sequence Synthetic oligonucleotide
tcctacctcctcccacctctt

SEQ ID NO: 83 - DNA, 20 bp, Artificial Sequence
  Description of Artificial Sequence Synthetic oligonucleotide
cctacctcctcccacctctt

SEQ ID NO: 84 - DNA, 19 bp, Artificial Sequence
  Description of Artificial Sequence Synthetic oligonucleotide
ctacctcctcccacctctt

SEQ ID NO: 85 - DNA, 20 bp, Artificial Sequence
  Description of Artificial Sequence Synthetic oligonucleotide
tcctcccacctcttgcctca

SEQ ID NO: 86 - DNA, 2060 bp, Homo sapiens
ctctatcgacgagtctggtagctgagcgttgggctgtaggtcgctgtgctgtgtgatcccccagagccatgcccgagatagtggatacctgttcgttggcctctccggcttccgtctgccggaccaagcacctgcacctgcgctgcagcgtcgactttactcgccggacgctgaccgggactgctgctctcacggtccagtctcaggaggacaatctgcgcagcctggttttggatacaaaggaccttacaatagaaaaagtagtgatcaatggacaagaagtcaaatatgctcttggagaaagacaaagttacaagggatcgccaatggaaatctctcttcctatcgctttgagcaaaaatcaagaaattgttatagaaatttcttttgagacctctccaaaatcttctgctctccagtggctcactcctgaacagacttctgggaaggaacacccatatctctttagtcagtgccaggccatccactgcagagcaatccttccttgtcaggacactccttctgtgaaattaacctatactgcagaggtgtctgtccctaaagaactggtggcacttatgagtgctattcgtgatggagaaacacctgacccagaagacccaagcaggaaaatatacaaattcatccaaaaagttccaataccctgctacctgattgctttagttgttggagctttagaaagcaggcaaattggcccaagaactttggtgtggtctgagaaagagcaggtggaaaagtctgcttatgagttttctgagactgaatctatgcttaaaatagcagaagatctgggaggaccgtatgtatggggacagtatgacctattggtcctgccaccatccttcccttatggtggcatggagaatccttgccttacttttgtaactcctactctactggcaggcgacaagtcactctccaatgtcattgcacatgaaatatctcatagctggacagggaatctagtgaccaacaaaacttgggatcacttttggttaaatgagggacatactgtgtacttggaacgccacatttgcggacgattgtttggtgaaaagttcagacattttaatgctctgggaggatggggagaactacagaattcggtaaagacatttggggagacacatcctttcaccaaacttgtggttgatctgacagatatagaccctgatgtagcttattcttcagttccctatgagaagggctttgctttacttttttaccttgaacaactgcttggaggaccagagattttcctaggattcttaaaagcttatgttgagaagttttcctataagagcataactactgatgactggaaggatttcctgtattcctattttaaagataaggttgatgttctcaatcaagttgattggaatgcctggctctactctcctggactgcctcccataaagcccaattatgatatgactctgacaaatgcttgtattgccttaagtcaaagatggattactgccaaagaagatgatttaaattcattcaatgccacagacctgaaggatctctcttctcatcaattgaatgagtttttagcacagacgctccagagggcacctcttccattggggcacataaagcgaatgcaagaggtgtacaacttcaatgccattaacaattctgaaatacgattcagatggctgcggctctgcattcaatccaagtgggaggacgcaattcctttggcgctaaagatggcaactgaacaaggaagaatgaagtttacccggcccttattcaaggatcttgctgcctttgacaaatcccatgatcaagctgtccgaacctaccaagagcacaaagcaagcatgcatcccgtgactgcaatgctggtggggaaagacttaaaagtggattaaagacctgcgtattgatgattttagagatttctcttttttaaatggaattcgtaaagaaatataaaacttcagctcacaattaaaactgtctttttagttttggctttttattgttttgttggtgattttactgaaataaagatgagctacttcttc

SEQ ID NO: 87 - protein, 611 aa, Homo sapiens
MPEIVDTCSLASPASVCRTKHLHLRCSVDFTRRTLTGTAALTVQSQEDNLRSLVLDTKDLTIEKVVINGQEVKYALGERQSYKGSPMEISLPIALSKNQEIVIEISFETSPKSSALQWLTPEQTSGKEHPYLFSQCQAIHCRAILPCQDTPSVKLTYTAEVSVPKELVALMSAIRDGETPDPEDPSRKIYKFIQKVPIPCYLIALVVGALESRQIGPRTLVWSEKEQVEKSAYEFSETESMLKIAEDLGGPYVWGQYDLLVLPPSFPYGGMENPCLTFVTPTLLAGDKSLSNVIAHEISHSWTGNLVTNKTWDHFWLNEGHTVYLERHICGRLFGEKFRHFNALGGWGELQNSVKTFGETHPFTKLVVDLTDIDPDVAYSSVPYEKGFALLFYLEQLLGGPEIFLGFLKAYVEKFSYKSITTDDWKDFLYSYFKDKVDVLNQVDWNAWLYSPGLPPIKPNYDMTLTNACIALSQRWITAKEDDLNSFNATDLKDLSSHQLNEFLAQTLQRAPLPLGHIKRMQEVYNFNAINNSEIRFRWLRLCIQSKWEDAIPLALKMATEQGRMKFTRPLFKDLAAFDKSHDQAVRTYQEHKASMHPVTAMLVGKDLKVD

SEQ ID NO: 88 - DNA, 4133 bp, Homo sapiens
ctggtcctgggtgtggggaagaaaggccatcaaggtagatgcgggtggggaacagcttgagagaggaggcaaggacaacccagtttctgtctgaaggggcctctggttgaccctggagtttctgtccccaaacacaggcctcacgggattctttctgtcctcatgcactgggcagaggttccttaacttcctttgttgcacattgccattctctcacatcccgtgcggtcaggaagcccttcctgaactctgacttcagttcttgctgcggtttctgcccatttttttcatatcctctgacagctgcgaggtcatctctgctctggcttttctccaagcagaacaagtgggggctctggaaaggttaagggacctcagtggccaccattatactttgcatctttcctgagaagtgagagttgaaagggaagcaggaaggcccatggtcagattgaaggaaggactttttagtttcttttttttttttttttttttttgagatggagtctcgctctgtcattcaggctggagtgcagtggtgcgatctcagctcactgcagcctccacttcctgggttcacatgattctcctgcctcagcctcccaagtagctgagactacaggcacatgccactacacccagctatcttttgtatttttagtagagacggggtttcaccatgttggccaggctggtctcaaactgctaacatcaagtgatctgctcccctcagcctcccaaagtgctgggattaccggtatgaaccaccacaacctgccaggaatttttagtttttagcttttgcaggagacttcaaggaaaggagacattcctctgtccaggaaacgggtaaggggaccatttctgcattgctggtttcccctcttggcagggtgggcatgaggcatcactgttcctgctccctcactcctgctcctcatgctcagcctgccagctcggcctcaactttgtgtgtctaaagtggaactgaatagtaggctgtgagaagataggaaagaggtagtgccaatctccttgcccagatcataaatccagactcagcagggtaaccacatgggcaagcacaaggtaggtgcttggggaaaggggaagtaattggcattctgtgtgataccaaggagaccatttggattttggcttctaccaaagagaatggagaattggttgacctaaatggaaccagtccctttaagtaaggggaggaaagggggtgctggaagatggccctcttcccaccacctagatcatagcttgaactgaagccaaggacagagtgctgcccccttcggcatttactgatgtgccctctttaaatcatgatgttatctaacccaaacccagacccaggacctagtcacagctccaacctacacttcctattaatcttaaaacaaagcaaaacaaaacaaaaagatatcagcattgtagcctccaatctgagcccatttcccttctctggctaccatacctccttctcctatatgataccattcactactttgttcaattatccagtctagacctgcatcttgaggccacacccagccttctcactccccacacccctctttcctctctcactgctccttcctggtctcttctcatctggccccacctctaaggagtcctcctgccttctgggttgccctggaaaacagactatcccccctcctagtgaagggagtgggtaggggtttcagccccaccctcaggaagatgcgtcttccctgtcctctgctctgtggtacttcctctctggctgatttagcaaacagcacctagacctggggccaggcctttggcagtgggacagatccagggataggctacaccaccctgccctgaccctgggattggcatcagcttccaaccagttcctgccaaagcttgtaagtcctcccgacggccatgaacactacatcttctgcagcacccccctcactaggtgtagagttcatctctctgctggctatcatcctgctgtcagtggcgctggctgtggggcttcccggcaacagctttgtggtgtggagtatcctgaaaaggatgcagaagcgctctgtcactgccctgatggtgctgaacctggccctggccgacctggccgtattgctcactgctccctttttccttcacttcctggcccaaggcacctggagttttggactggctggttgccgcctgtgtcactatgtctgcggagtcagcatgtacgccagcgtcctgcttatcacggccatgagtctagaccgctcactggcggtggcccgcccctttgtgtcccagaagctacgcaccaaggcgatggcccggcgggtgctggcaggcatctgggtgttgtcctttctgctggccacacccgtcctcgcgtaccgcacagtagtgccctggaaaacgaacatgagcctgtgcttcccgcggtaccccagcgaagggcaccgggccttccatctaatcttcgaggctgtcacgggcttcctgctgcccttcctggctgtggtggccagctactcggacatagggcgtcggctacaggcccggcgcttccgccgcagccgccgcaccggccgcctggtggtgctcatcatcctgaccttcgccgccttctggctgccctaccacgtggtgaacctggctgaggcgggccgcgcgctggccggccaggccgccgggttagggctcgtggggaagcggctgagcctggcccgcaacgtgctcatcgcactcgccttcctgagcagcagcgtgaaccccgtgctgtacgcgtgcgccggcggcggcctgctgcgctcggcgggcgtgggcttcgtcgccaagctgctggagggcacgggctccgaggcgtccagcacgcgccgcgggggcagcctgggccagaccgctaggagcggccccgccgctctggagcccggcccttccgagagcctcactgcctccagccctctcaagttaaacgaactgaactaggcctggtggaaggaggcgcactttcctcctggcagaatgctagctctgagccagttcagtacctggaggaggagcaggggcgtggagggcgtggagggcgtgggagcgtgggaggcgggagtggagtggaagaagagggagaggtggagcaaagtgagggccgagtgagagcgtgctccagcctggctcccacaggcagctttaaccattaaaactgaagtctgaaatttggtcaaccttgtgagtggggtacatgtgctgtgggtatcggggtgctcgtgggcgccctggtggggcccctctcggtagttgagagtcacgtcctttagttccccatgatttacaattttggaagggacacaaagaaacatagacttcccccatcccagatgattccgagtacatagtctgcagataatacttagcaaaacgcagtctacagactcctaaagcagcttgtctaggaagaccacccatgtgggcttatcactccaggttctgtgacccgggaccttctgagaaaacagcactgctgtgaaatatcttccttgaagcctgtgataagtctccttgttagaatgactccaacttcctgccaataatctttgtcctctccataggagatgttctaggggatgccttccttccctccatttcacaaagaggccagacttgaggactaagtcattagatcttatcccttagaattttgcatatcagcatctgctaacctccacaacacaccctggcacagggtggggctgagggccccaggaaacaaagattcccaaaagtgagagggatgagtcattatttcctagagatgactgttgttttagagaaccttggtccaactctgttctgacaaggttttaggaagatggcaacaacagtggcagcagtgtactttttggatctttctcataaaaaaacaaaagagcaagtaagagagggaaaccaaatatccacatgcaacatccgcaacaaatctagtatgtcaaggtttgacatactcccatggaccccaaagtatgagccagtgagaatgagtcatcaatatctcaagacccatataccagcatctgtgcagaaggatgcagaaggaagcaaagggatgttggatggacctaagaagaggagatccc

SEQ ID NO: 89 - protein, 352 aa, Homo sapiens
MNTTSSAAPPSLGVEFISLLAIILLSVALAVGLPGNSFVVWSILKRMQKRSVTALMVLNLALADLAVLLTAPFFLHFLAQGTWSFGLAGCRLCHYVCGVSMYASVLLITAMSLDRSLAVARPFVSQKLRTKAMARRVLAGIWVLSFLLATPVLAYRTVVPWKTNMSLCFPRYPSEGHRAFHLIFEAVTGFLLPFLAVVASYSDIGRRLQARRFRRSRRTGRLVVLIILTFAAFWLPYHVVNLAEAGRALAGQAAGLGLVGKRLSLARNVLIALAFLSSSVNPVLYACAGGGLLRSAGVGFVAKLLEGTGSEASSTRRGGSLGQTARSGPAALEPGPSESLTASSPLKLNELN

SEQ ID NO: 90 - DNA, 2798 bp, Homo sapiens
aaactggccctggccctgaaccaaataccttgaaccctcgtaaactccataccctgacccccttgttttggatatacccaggtagaacaactctctctcactgtctgttgtgaggatacgctgtagcccactcattaagtacattctcctaataaatgctttggactgatcaccctgccagtcttttgtcttgggcaatctatacttttctcagaggttcccaaggcctactgaagggacttaacatactcttaatggctttcctctctcttgttttaccttatgccctcacttcctgagttaacctcccaaatacaggatcacctgtacccaagcccttagctcaagaatacaggatcacctgtacccaagcccttagctcaagctctgctttggaagaacccaaactaagacagtgctcctggtgccctccccaagcaacctcaagttctggctgttacttgagcagaggcctttcttttcccttcccccagctctatccatctgccaggcccccctcaaatctcttcatttccaagttttgcttgacttttccaagaggagagggctgcttcttagtatgtccctactcatcctttcctttcttgtcttgtatcctggtgcagcctggtaatggggcctcttcatggttgtgtgtcatgactccctaaccattatgcctccatgcatcccctgttcctcctggaacctagcaccatgccttacatggaaaagctgtcattgacagcccggtgagagccctgagggtggagtgactggggcagggcctgaggcaagaggtgggaggaggtaggaggccaggggctcagccggaccaggagactggaaacaggcaaggataaggcaggtgggggactgagttgtttgggtcacctctgcaggccagagagaccaggcaacatacacactgcagaaggtgggctgggaggattggggccagagctgggggagggatgagaacagaagcaggaccaggattcagcagagtcctcctatttccttccaccaccagggaatcttactgccccacttcagcttgtgctgtttcctggcaaggcaggctctcacatgcctggacgcctgggtgcgttggtgatgggaaggagcagggtgagggaggggccccaggagaggcccaggatgagcctcatcttgtccctccccattcttgtcttaccctctgcaaatgtgataggcacaggacaggagtaggcacctcgcctactgctgcttaacctttcagcttctccaggcccccaatcctgcttgctcccagcttggtaagtagatctgtgcacgtccctttacaccccaccatccagttttgcccagatgtgctagaatggggctggacaaagaaggaggggccagactagaggagtggtggtagagatagtgacagcctggggtgaggactttatgcctgtttaccactgagctctgggaaggaggccaggagtggggcaggtcaactgactgggagcaggggatctgggttccaagaaggagttgtgtttgaggtggggtctgggtcctcgtggaagtcaggactcccaggcagaaaagaggcaggctgcagggaagtaaggaggaggcatggcaccttctcatcgggcatcacaggtggggttttgccccacccctgaacgccctctgtggcgccttccacccacctgtaggcccagaaggatgtcggtctgctaccgtcccccagggaacgagacactgctgagctggaagacttcgcgggccacaggcacagccttcctgctgctggcggcgctgctggggctgcctggcaacggcttcgtggtgtggagcttggcgggctggcggcctgcacgggggcgaccgctggcggccacgcttgtgctgcacctggcgctggccgacggcgcggtgctgctgctcacgccgctctttgtggccttcctgacccggcaggcctggccgctgggccaggcgggctgcaaggcggtgtactacgtgtgcgcgctcagcatgtacgccagcgtgctgctcaccggcctgctcagcctgcagcgctgcctcgcagtcacccgccccttcctggcgcctcggctgcgcagcccggccctggcccgccgcctgctgctggcggtctggctggccgccctgttgctcgccgtcccggccgccgtctaccgccacctgtggagggaccgcgtatgccagctgtgccacccgtcgccggtccacgccgccgcccacctgagcctggagactctgaccgctttcgtgcttcctttcgggctgatgctcggctgctacagcgtgacgctggcacggctgcggggcgcccgctggggctccgggcggcacggggcgcgggtgggccggctggtgagcgccatcgtgcttgccttcggcttgctctgggccccctaccacgcagtcaaccttctgcaggcggtcgcagcgctggctccaccggaaggggccttggcgaagctgggcggagccggccaggcggcgcgagcgggaactacggccttggccttcttcagttctagcgtcaacccggtgctctacgtcttcaccgctggagatctgctgccccgggcaggtccccgtttcctcacgcggctcttcgaaggctctggggaggcccgagggggcggccgctctagggaagggaccatggagctccgaactacccctcagctgaaagtggtggggcagggccgcggcaatggagacccggggggtgggatggagaaggacggtccggaatgggacctttgacagcagaccct

SEQ ID NO: 91 - protein, 389 aa, Homo sapiens
MAPSHRASQVGFCPTPERPLWRLPPTCRPRRMSVCYRPPGNETLLSWKTSRATGTAFLLLAALLGLPGNGFVVWSLAGWRPARGRPLAATLVLHLALADGAVLLLTPLFVAFLTRQAWPLGQAGCKAVYYVCALSMYASVLLTGLLSLQRCLAVTRPFLAPRLRSPALARRLLLAVWLAALLLAVPAAVYRHLWRDRVCQLCHPSPVHAAAHLSLETLTAFVLPFGLMLGCYSVTLARLRGARWGSGRHGARVGRLVSAIVLAFGLLWAPYHAVNLLQAVAALAPPEGALAKLGGAGQAARAGTTALAFFSSSVNPVLYVFTAGDLLPRAGPRFLTRLFEGSGEARGGGRSREGTMELRTTPQLKVVGQGRGNGDPGGGMEKDGPEWDL